(19) United States
(12) Patent Application Publication
(10) Pub. No.: US 2010/0318108 A1
(43) Pub. Date: Dec. 16, 2010

(54) COMPOSITE MESH DEVICES AND METHODS FOR SOFT TISSUE REPAIR

(75) Inventors: Arindam Datta, Hillsborough, NJ (US); Craig Friedman, Santa Fe, NM (US); Lawrence P. Lavelle, JR., Rahway, NJ (US); Gene Park, Santa Rosa, CA (US); Dave Pearce, Los Gatos, CA (US); Rujul B. Majmundar, Belle Mead, NJ (US)

Correspondence Address: KING & SPALDING, 1185 AVENUE OF THE AMERICAS, NEW YORK, NY, 10036-4003, US

(73) Assignee: Biomerix Corporation, New York, NY (US)

(21) Appl. No.: 12/699,012

(22) Filed: Feb. 2, 2010

Related U.S. Application Data
(60) Provisional application No. 61/149,333, filed on Feb. 2, 2009.

(51) Int. Cl. A61B 17/00; D03D 19/00; B32B 37/12; B32B 37/00

(52) U.S. Cl. 606/151; 442/1; 156/60; 156/299

(57) ABSTRACT

A composite implantable device for promoting tissue ingrowth therein comprising a biodurable reticulated elastomeric matrix having a three-dimensional porous structure having a continueous network of interconnected and intercommunicating open pores and a support structure is disclosed. The support structure may be a polymeric surgical mesh comprising a plurality of intersecting one-dimensional reinforcement elements, wherein said mesh is affixed to a face of said first matrix. Methods of making and using the implantable device are also provided.

This application claims priority under 35 U.S.C. §119(e) to U.S. Provisional Patent Application Ser. No. 61/149,333, filed Feb. 2, 2009, the disclosures of which are hereby incorporated by reference herein.

### FIELD OF THE INVENTION

This invention relates to composite mesh devices intended for repair of soft tissue defects, comprising a novel biodurable reticulated elastomeric matrix which is designed to support tissue ingrowth and at least one functional element.

### BACKGROUND OF THE INVENTION

Presently available hernia devices are made from synthetic components which are polypropylene, polyester, or expanded poly(tetrafluoroethylene)) (“ePTFE”) formed into a two dimensional shape or from biological sources such as decullarized human cadaver skin or from animal sources such as porcine or bovine collagen. Currently, there is no complete solution to the repair of soft tissue defects, specifically inguinal, femoral, incisional, umbilical, and epigastric hernias.

There is an ongoing need for an improved method of treatment of a soft tissue defect, such as a hernia.

### SUMMARY OF THE INVENTION

A composite implantable device for promoting tissue ingrowth therein is provided, comprising (i) a first biodurable reticulated elastomeric matrix having a three-dimensional porous structure comprising a continuous network of interconnected and intercommunicating open pores, and (ii) a polymeric surgical mesh comprising a plurality of intersecting one-dimensional reinforcement elements. The mesh is affixed to a face of the first matrix. Preferably, the first matrix comprises polycarbonate polyurethane or polycarbonate polyurethane-urea. In some embodiments, the mesh may comprise an absorbable or non-resorbable material. Preferably, the mesh comprises knitted polypropylene monofilament fibers. In other embodiments, the composite implantable device may further comprise a second biodurable reticulated elastomeric matrix having a three-dimensional porous structure comprising a continuous network of interconnected and intercommunicating open pores. The mesh is sandwiched between said first and second matrices. In another embodiment, the device comprises a polymeric film coating the first matrix or the mesh. The coating reduces adhesion of the device to biologic surfaces. The polymeric film comprises poly (L-lactide co ε-caprolactone).

A method for treating a hernia is provided. The method includes making an incision into an affected area, placing the composite implantable device onto the affected area, and securing the device to the affected area.

In another embodiment, a method for manufacturing a composite implantable device is provided. The method includes preparing a biodurable reticulated elastomeric matrix having a three-dimensional porous structure comprising a continuous network of interconnected and intercommunicating open pores, applying an adhesive to a polymeric surgical mesh, and affixing the mesh to a face of the matrix to form the composite implantable device. The mesh comprises a plurality of intersecting one-dimensional reinforcement elements.

These and other aspects of the present invention will become apparent to those skilled in the art after a reading of the following detailed description of the invention, including the figures and appended claims.

### BRIEF DESCRIPTION OF THE FIGURES

Some embodiments of the invention, and of making and using the invention, are described in detail below, which description is to be read with and in the light of the foregoing description, by way of example, with reference to the accompanying drawings in which:

FIG. 1 is a schematic view showing one possible morphology for a portion of the microstructure of one embodiment of a porous biodurable elastomeric product according to the invention;

FIG. 2 is a schematic block flow diagram of a process for preparing a porous biodurable elastomeric implantable device according to the invention;

FIG. 3 illustrates an exemplary schematic of the “sandwich design” or a composite elastomeric matrix with 2-dimensional mesh reinforcement;

FIG. 4 illustrates schematic of manufacturing of the “sandwich design” or a composite elastomeric matrix with 2-dimensional mesh reinforcement;

FIG. 5 illustrates several different exemplary reticulated elastomeric matrix reinforcement grids;

FIG. 6 illustrates several different exemplary reticulated elastomeric matrix reinforcement grids;

FIG. 7 illustrates exemplary reticulated elastomeric matrix 2-dimensional reinforcement grid;

FIG. 8 illustrates an exemplary schematic of a 2-dimensional mesh reinforcement attached to one layer of elastomeric matrix using an adhesive and a film of biocompatible polymer acting as anti-adhesive coating.

FIG. 9 illustrates schematic of manufacturing dimensional mesh reinforcement attached to one layer of elastomeric matrix using an adhesive and a film of biocompatible polymer acting as anti-adhesive coating;

FIG. 10 illustrates the geometry of the suture pullout strength test;

FIG. 11 shows a histology analysis photograph of the device of Example 3;

FIG. 12 a histology analysis photograph of the device of Example 5;

FIG. 13 is a scanning electron micrograph image of Reticulated Elastomeric Matrix 2;

FIGS. 14a-14c are photographic examples of Surgical Mesh for embodiments of the invention;

FIGS. 15a-15c are photographic examples of Surgical Mesh With Coatings Cross Section SEM for embodiments of the invention;

FIGS. 16a-16h are photographic examples of “Double sided Biomerix Mesh bonded to a polypropylene mesh with a silicone adhesive” for embodiments of the invention;

FIGS. 17a and 17b are photographic examples of “Biomerix Matrix with anti-adhesion coating” for embodiments of the invention;

FIG. 18 is a photographic example of Porous Structure (SEM) for embodiments of the invention;

FIG. 19 is a flow chart showing an exemplary process flow diagram for an exemplary embodiment of the invention;

FIGS. 20a-20f are photographic illustrations of examples of microscope evaluations from in vivo testing in rat models at various time points shown in 40× magnification; and

FIGS. 21a-21d are photographic illustrations of microscope evaluations at 26 weeks from in vivo testing in rat models at 26 weeks shown in 4×, 10×, 20× and 40× magnification.

### DETAILED DESCRIPTION OF THE INVENTION

Reference will now be made in detail to embodiments of the invention, one or more examples of which are illustrated in the accompanying drawings. Each example is provided by way of explanation of the invention, not as a limitation of the invention. It will be apparent to those skilled in the art that various modifications and variations can be made in the present invention without departing from the scope or spirit of the invention. For example, features illustrated or described as part of one embodiment can be used on another embodiment to yield a still further embodiment. Thus, it is intended that the present invention cover such modifications and variations that come within the scope of the invention.

The inventive implantable device for repair of soft tissue defects generally includes a biodurable, reticulated elastomeric matrix comprising a plurality of pores (the pores may be interconnected and intercommunicating open pores, forming a network that permits tissue in-growth and proliferation into the implant) and a support structure for reinforcing the mechanical properties of the device. In addition, the implantable device for embodiments of the invention can be formed from two or more individual reticulated elastomeric matrices. The implantable device according to the present invention is particular useful for surgical repair of hernias. Certain embodiments of the invention provide a complete solution to the repair of soft tissue defects, specifically inguinal, femoral, ventral, incisional, umbilical, and epigastric hernias.

### Biodurable Reticulated Matrix

A first component of the implantable device of the present invention is a reticulated elastomeric matrix. The reticulated elastomeric matrix for embodiments of the invention comprises a network of cells which forms a three-dimensional spatial structure. The cells communicate and connect to each other via the open-celled pores contained within the cells. This network results in a matrix with a unique morphology, composed of continuous interconnected and intercommunicating pores. The reticulated elastomeric matrix permits tissue in-growth and proliferation into the implant. Preferably, the reticulated elastomeric matrix is biodurable. In an exemplary embodiment, the reticulated elastomeric matrix may be resiliently compressible and may preferably comprise polycarbonate polyurethane or polycarbonate polyurethane urea. Suitable matrices include, without limitation, those described in U.S. Patent Application Publication No. 2007/0190108, the disclosures of which are hereby incorporated by reference.

Because of the biointegrative three dimensional porous structure characteristics of the reticulated elastomeric matrix, embodiments of the invention have the advantage of potentially better and faster tissue in-growth, healing, and remodeling.

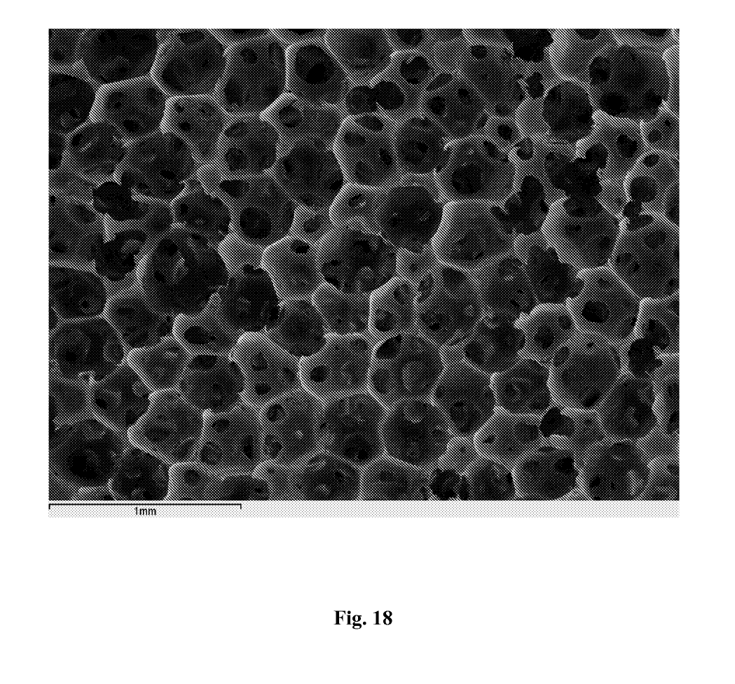

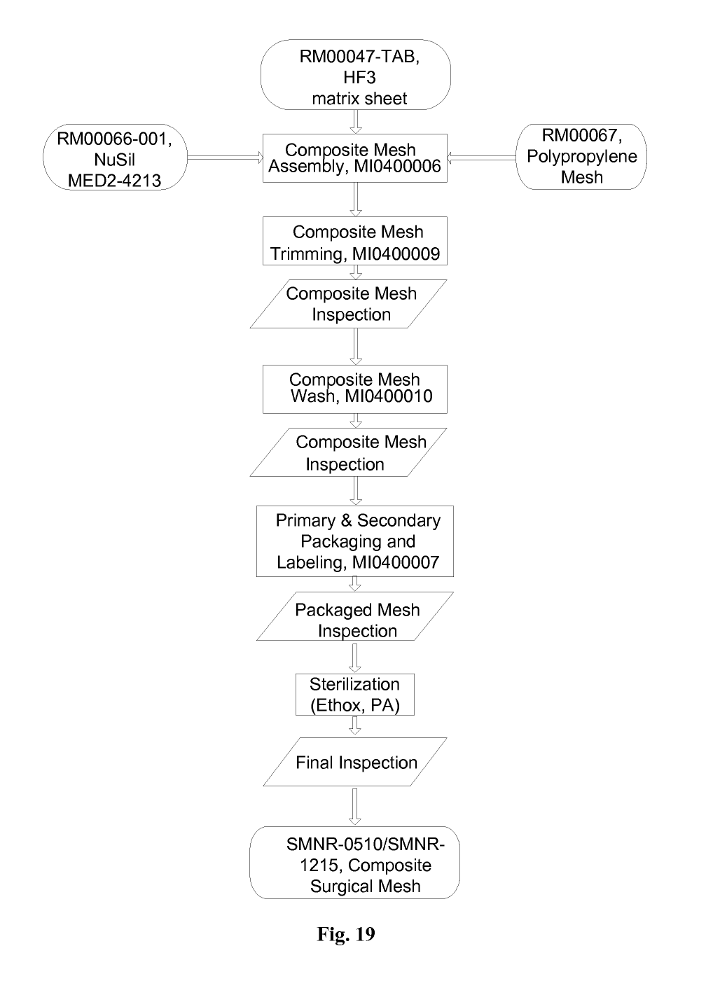

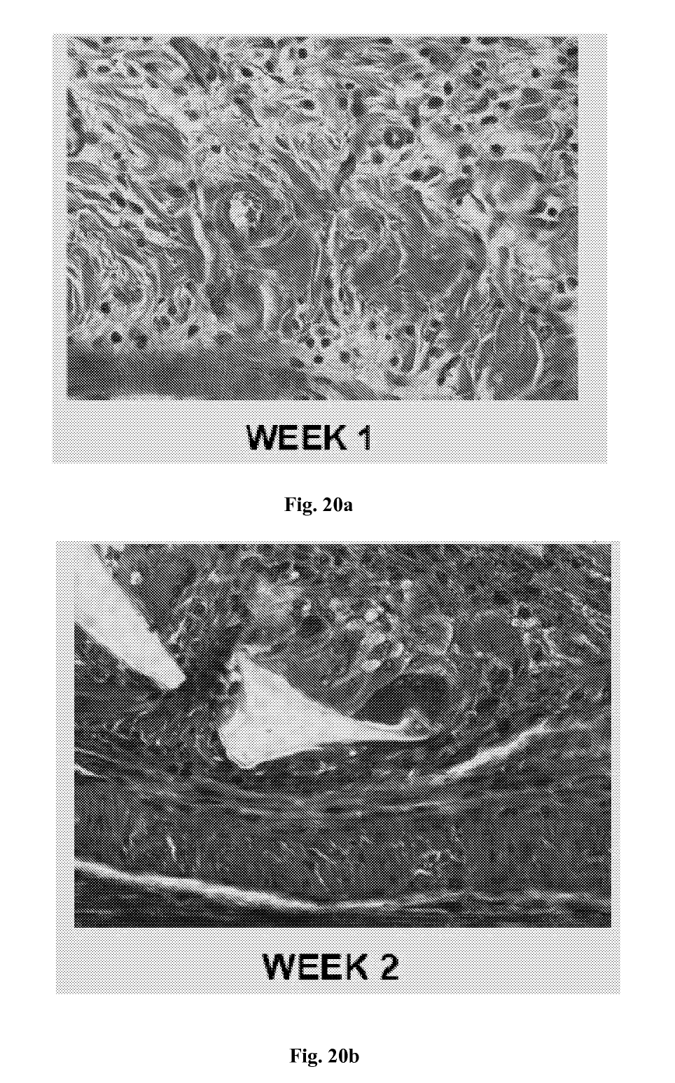

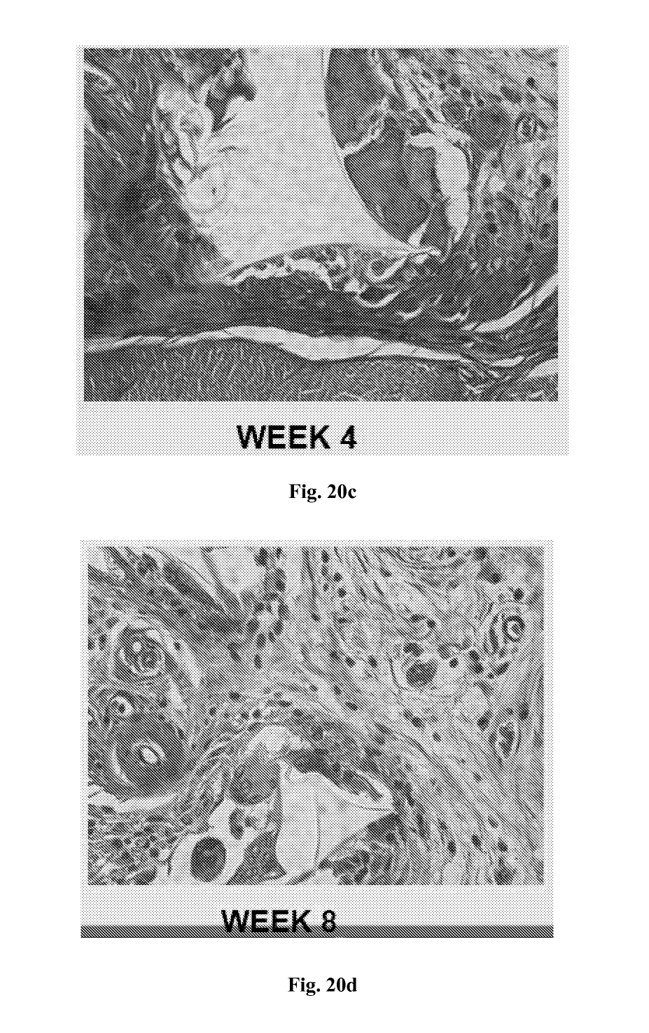

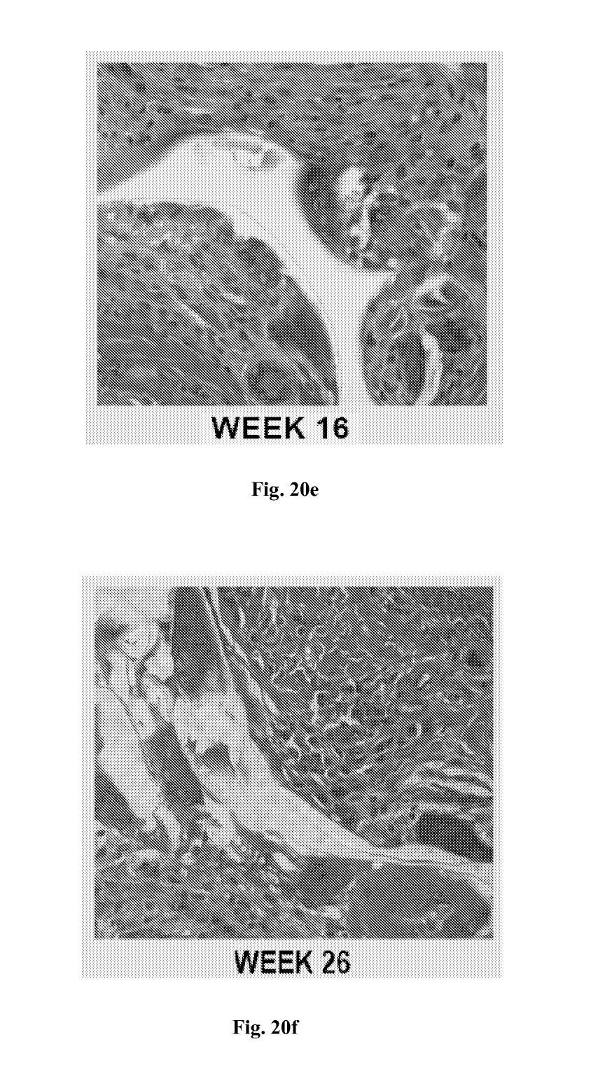

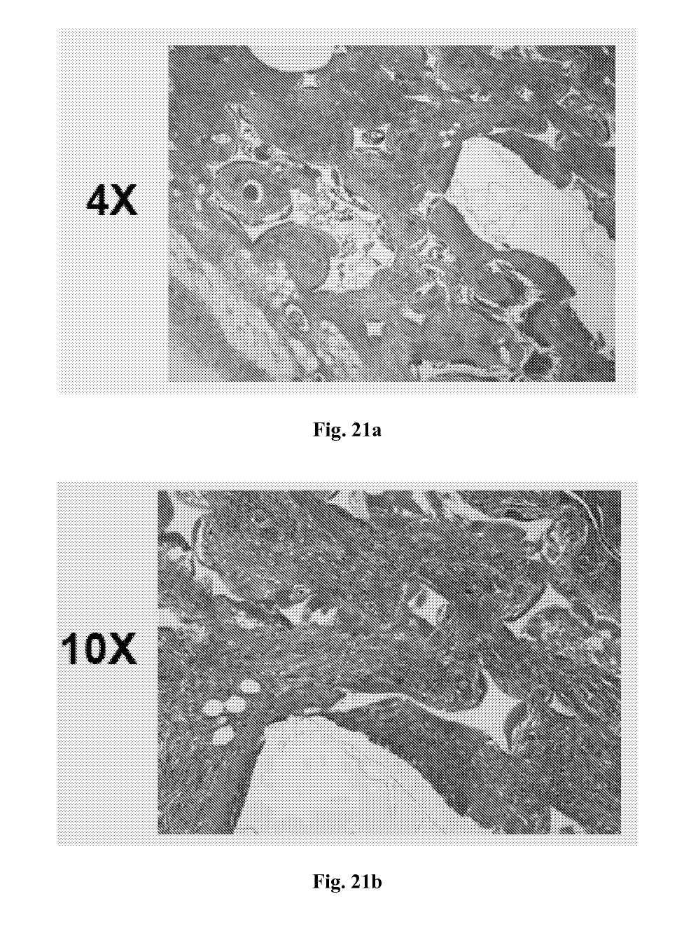

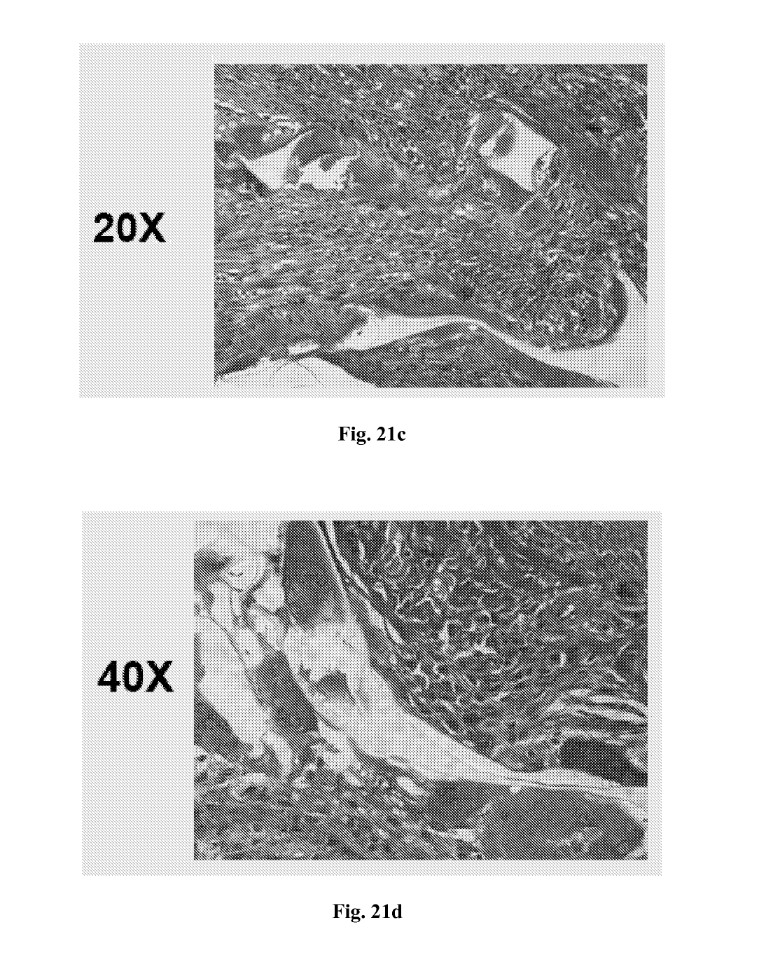

FIG. 18 is a photographic illustration that illustrates an example of the porous structure for embodiments of the invention.

Certain embodiments of the invention comprise reticulated biodurable elastomer products, which are also compressible and exhibit resilience in their recovery, that have a diversity of applications and can be employed, by way of example, in biological implantation, especially into humans, for long-term TE implants, especially but not limited to where dynamic loadings and/or extensions are experienced, such as in soft tissue related orthopedic applications; repair of soft tissue defects, specifically inguinal, femoral, ventral, incisional, umbilical, and epigastric hernias; surgical meshes for tissue augmentation, support and repair; for therapeutic purposes; for cosmetic, reconstructive, urologic or gastroesophageal purposes; or as substrates for pharmaceutically-active agent, e.g., drug, delivery. Other embodiments involve reticulated biodurable elastomer products for in vivo delivery via catheter, endoscope, arthoscope, laproscop, cystoscope, syringe or other suitable delivery-device and can be satisfactorily implanted or otherwise exposed to living tissue and fluids for extended periods of time, for example, at least 29 days.

It would be desirable to form implantable devices suitable for use as tissue engineering scaffolds, or other comparable substrates, to support in vivo cell propagation applications, for example in a large number of orthopedic applications especially in soft tissue attachment, in repair of soft tissue defects such as number of hernia applications, surgical meshes for augmentation, support and ingrowth of a prosthetic organ. Without thout being bound by any particular theory, the reticulated implantable devices having a high void content and a high degree of reticulation allowing unfettered acccess to the inter-connected and inter-communicating high void content is thought to allow the implantable device to become at least partially ingrown and/or proliferated, in some cases substantially ingrown and proliferated, in some cases completely ingrown and proliferated, with cells including tissues such as fibroblasts, fibrous tissues, scar tissues, endothelial cells, synovial cells, bone marrow stromal cells, stem cells and/or fibrocartilage cells. The ingrown and/or proliferated tissues thereby provide functionality, such as load bearing capability, for defect repair of the original tissue that is being repaired or replaced. However, prior to the advent of the present invention, materials and/or products meeting the requirements for such implantable devices have not been available.

Because of the biointegrative three dimensional inter-connected and inter-communicating structure characteristics of the base reticulated implantable devices, embodiments of the invention have the advantage of potentially better and faster tissue in-growth, healing, and remodeling.

Broadly stated, certain embodiments of the reticulated biodurable elastomeric products of the invention comprise, or are largely if not entirely, constituted by a highly permeable, reticulated matrix formed of a biodurable polymeric elastomer that is resiliently-compressible so as to regain its shape after delivery to a biological site. In one embodiment, the elastomeric matrix has good fatigue resistance associated with dynamic loading. In another embodiment, the elastomeric matrix is chemically well-characterized. In another embodiment, the elastomeric matrix is physically well-characterized. In another embodiment, the elastomeric matrix is chemically and physically well-characterized.

Certain embodiments of the invention can support cell growth and permit cellular ingrowth and proliferation in vivo and are useful as in vivo biological implantable devices, for example, for tissue engineering scaffolds that may be used in vitro or in vivo to provide a substrate for cellular propagation.

The implantable devices of the invention are useful for many applications as long-term tissue engineering implantssuch as in repair and regeneration of soft tissue related orthopedic applications, in repair of soft tissue defects such as number of hernia applications and is the use of surgical meshes for regeneration, augmentation, etc. Other embodiments of the invention provide composite mesh comprising a novel biodurable reticulated elastomeric matrix which is designed to support tissue ingrowth and at least one functional element for the intended for repair of soft tissue defects related orthopedic applications and in the repair of soft tissue defects such as number of hernia applications; specifically inguinal, femoral, ventral, incisional, umbilical, and epigastric hernias. In one embodiment, the functional element is a reinforcing elment that can be fiber or a mesh designed to enhance the mechanical load bearing fucntions such as strength, stiffnes, tear resistance, burst strength, suture pull out strength, etc. Such reinforceemnts may either be permanent (e.g., polyester, polypropylene, teflon, etc) or resorbable (copolymers and homopolymers of polylactic acid, poly glycolic acid, polycapralactone, polyparadioxanone, etc.). In other embodiments, the functional element is a thin layer, coating or film of either a permanent polymer or biodegradable polymer or a bioactive polymer or a biopolymer or biologically derived collagen used to reduce the potential for adhesions, reduce the potential for biological adhesions and enhance tissue response. In yet another embodiment, the functional element is a polymeric and/or metallic structures used to impart shape memory; and markers including dyes used to differentiate between two sides of a mesh which may have differing characteristics. In one embodiment, one or all or at least a selected number of the functional elements can be incorporated into the biodurable reticulated elastomeric matrix. Any of these preferred functional elements may be incorporated with the biodurable reticulated elastomeric matrix using various processing techniques known in the art including adhesive bonding, melt processing, compression molding, solution casting, thermal bonding, suturing, and other techniques.

In one embodiment, the reticulated elastomeric matrix of the invention facilitates tissue ingrowth by providing a surface for cellular attachment, migration, proliferation and/or coating (e.g., collagen) deposition. In another embodiment, any type of tissue can grow into an implantable device comprising a reticulated elastomeric matrix of the invention, including, by way of example, epithelial tissue (which includes, e.g., squamous, cuboidal and columnar epithelial tissue), connective tissue (which includes, e.g., areolar tissue, dense regular and irregular tissue, reticular tissue, adipose tissue, cartilage and bone), and muscle tissue (which includes, e.g., skeletal, smooth and cardiac muscle), or any combination thereof, e.g., fibrovascular tissue.

The structure, morphology and properties of the elastomeric matrices of this invention can be engineered or tailored over a wide range of performance by varying the starting materials and/or the processing and/or the post processing conditions for different functional or therapeutic uses. In another embodiment, the structure, morphology and properties of the composite mesh comprising elastomeric matrices and at least one functional element can be engineered or tailored over a wide range of performance by varying the starting materials and/or the processing and/or the post processing conditions.

In one embodiment, elastomeric matrices of the invention have sufficient resilience to allow substantial recovery, e.g., to at least about 50% of the size of the relaxed configuration in at least one dimension, after being compressed for implantation in the human body, for example, a low compression set, e.g., at 25° C. or 37° C., and sufficient strength and flow-through for the matrix to be used for controlled release of pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment, elastomeric matrices of the invention have sufficient resilience to allow recovery to at least about 75% of the size of the relaxed configuration in at least one dimension after being compressed for implantation in the human body. In another embodiment, elastomeric matrices of the invention have sufficient resilience to allow recovery to at least about 90% of the size of the relaxed configuration in at least one dimension after being compressed for implantation in the human body. In another embodiment, elastomeric matrices of the invention have sufficient resilience to allow recovery to at least about 95% of the size of the relaxed configuration in at least one dimension after being compressed for implantation in the human body.

In the present application, the term “biodurable” describes elastomers and other products that are stable for extended periods of time in a biological environment. Such products should not exhibit significant symptoms of breakdown or degradation, erosion or significant deterioration of mechanical properties relevant to their employment when exposed to biological environments for periods of time commensurate with the use of the implantable device. The period of implantation may be weeks, months or years; the lifetime of a host product in which the elastomeric products of the invention are incorporated, such as a graft or prosthetic; or the lifetime of a patient host to the elastomeric product. In one embodiment, the desired period of exposure is to be understood to be at least about 29 days. In another embodiment, the desired period of exposure is to be understood to be at least 29 days. In one embodiment, the implantable device is biodurable for at least 2 months. In another embodiment, the implantable device is biodurable for at least 6 months. In another embodiment, the implantable device is biodurable for at least 12 months. In another embodiment, the implantable device is biodurable for longer than 12 months. In another embodiment, the implantable device is biodurable for at least 24 months. In another embodiment, the implantable device is biodurable for at least 5 years. In another embodiment, the implantable device is biodurable for longer than 5 years.

In one embodiment, biodurable products of the invention are also biocompatible. In the present application, the term “biocompatible” means that the product induces few, if any, adverse biological reactions when implanted in a host patient. Similar considerations applicable to “biodurable” also apply to the property of “biocompatibility”.

An intended biological environment can be understood to in vivo, e.g., that of a patient host into which the product is implanted or to which the product is topically applied, for example, a mammalian host such as a human being or other primate, a pet or sports animal, a livestock or food animal, or a laboratory animal. All such uses are contemplated as being within the scope of the invention.

In one embodiment, structural materials for the inventive biodurable reticulatd elastomers are synthetic polymers, especially but not exclusively, elastomeric polymers that are resistant to biological degradation, for example, in one embodiment, polycarbonate polyurethanes, polycarbonate urea-urethanes, poly(carbonate-co-ether) urea-urethanes, polysiloxanes and the like, in another embodiment polycarbonate polyurethanes, polycarbonate urea-urethanes, polycarbonate polysiloxane polyurethanes, polycarbonate polysiloxane urea-urethanes, and polysiloxanes, in another embodiment polycarbonate polyurethanes, polycarbonate urea-urethanes, and polysiloxanes. Such elastomers are generally hydrophobic but, pursuant to the invention, may be treated to have surfaces that are less hydrophobic or somewhat hydrophilic. In another embodiment, such elastomers may be produced with surfaces that are significantly or largely-hydrophobic.

The reticulated biodurable elastomeric products of the invention can be described as having a “macrostructure” and a “microstructure”, which terms are used herein in the general senses described in the following paragraphs.

The “macrostructure” refers to the overall physical characteristics of an article or object formed of the biodurable elastomeric product of the invention, such as: the outer periphery as described by the geometric limits of the article or object, ignoring the pores or voids; the “macrostructural surface area” which references the outermost surface areas as though any pores thereon were filled, ignoring the surface areas within the pores; the “macrostructural volume” or simply the “volume” occupied by the article or object which is the volume bounded by the macrostructural, or simply “macro”, surface area; and the “bulk density” which is the weight per unit volume of the article or object itself as distinct from the density of the structural material.

The “microstructure” refers to the features of the interior structure of the biodurable elastomeric material from which the inventive products are constituted such as pore dimensions; pore surface area, being the total area of the material surfaces in the pores; and the configuration of the struts and intersections that constitute the solid structure of certain embodiments of the inventive elastomeric product.

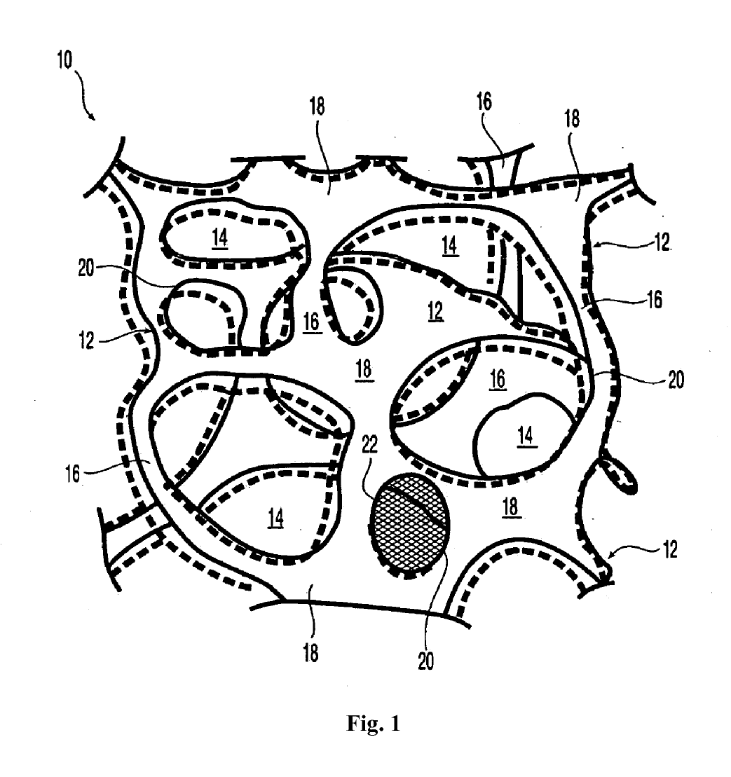

Referring to FIG. 1, what is shown for convenience is a schematic depiction of the particular morphology of a reticulated matrix. FIG. 1 is a convenient way of illustrating some of the features and principles of the microstructure of some embodiments of the invention. This figure is not intended to be an idealized depiction of an embodiment of, nor is it a detailed rendering of a particular embodiment of the elastomeric products of the invention. Other features and principles of the microstructure will be apparent from the present specification, or will be apparent from one or more of the inventive processes for manufacturing porous elastomeric products that are described herein.

Morphology

Described generally, the microstructure of the illustrated porous biodurable elastomeric matrix 10, which may, inter alia, be an individual element having a distinct shape or an extended, continuous or amorphous entity, comprises a reticulated solid phase 12 formed of a suitable biodurable elastomeric material and interspersed therewithin, or defined thereby, a continuous interconnected void phase 14, the latter being a principle feature of a reticulated structure.

In one embodiment, the elastomeric material of which elastomeric matrix 10 is constituted may be a mixture or blend of multiple materials. In another embodiment, the elastomeric material is a single synthetic polymeric elastomer such as will be described in more detail below. In other embodiments, although elastomeric matrix 10 is subjected to post-reticulation processing, such as annealing, compressive molding and/or reinforcement, it is to be understood that the elastomeric matrix 10 retains its defining characteristics, that is, it remains biodurable, reticulated and elastomeric.

Void phase 14 will usually be air- or gas-filled prior to use. During use, void phase 14 will in many but not all cases become filled with liquid, for example, with biological fluids or body fluids.

Solid phase 12 of elastomeric matrix 10, as shown in FIG. 1, has an organic structure and comprises a multiplicity of relatively thin struts 16 that extend between and interconnect a number of intersections 18. The intersections 18 are substantial structural locations where three or more struts 16 meet one another. Four or five or more struts 16 may be seen to meet at an intersection 18 or at a location where two intersections 18 can be seen to merge into one another. In one embodiment, struts 16 extend in a three-dimensional manner between intersections 18 above and below the plane of the paper, favoring no particular plane. Thus, any given strut 16 may extend from an intersection 18 in any direction relative to other struts 16 that join at that intersection 18. Struts 16 and intersections 18 may have generally curved shapes and define between them a multitude of pores 20 or interstitial spaces in solid phase 12. Struts 16 and intersections 18 form an interconnected, continuous solid phase.

As illustrated in FIG. 1, the structural components of the solid phase 12 of elastomeric matrix 10, namely struts 16 and intersections 18, may appear to have a somewhat laminar configuration as though some were cut from a single sheet, it will be understood that this appearance may in part be attributed to the difficulties of representing complex three-dimensional structures in a two dimensional figure. Struts 16 and intersections 18 may have, and in many cases will have, non-laminar shapes including circular, elliptical and non-circular cross-sectional shapes and cross sections that may vary in area along the particular structure, for example, they may taper to smaller and/or larger cross sections while traversing along their longest dimension.

The cells of elastomeric matrix 10 are formed from clusters or groups of pores 20, which would form the walls of a cell except that the cell walls 22 of most of the pores 20 are absent or substantially absent owing to reticulation. In particular, a small number of pores 20 may have a cell wall of structural material also called a “window” or “window pane” such as cell wall 22. Such cell walls are undesirable to the extent that they obstruct the passage of fluid and/or propagation and proliferation of tissues through pores 20. Cell walls 22 may, in one embodiment, be removed in a suitable process step, such as reticulation as discussed below.

The individual cells forming the reticulated elastomeric matrix are characterized by their average cell diameter or, for nonspeherical cells, by their largest transverse dimension. The reticulated elastomeric matrix comprises a network of cells that form a three-dimensional spatial structure or void phase 14 which is interconnected via the open pores 20 therein. In one embodiment, the cells form a 3-dimensional superstructure. The pores provide connectivity between the individual cells, or between clusters or groups of pores which form a cell.

Except for boundary terminations at the macrostructural surface, in the embodiment shown in FIG. 1 solid phase 12 of elastomeric matrix 10 comprises few, if any, free-ended, dead-ended or projecting “strut-like” structures extending from struts 16 or intersections 18 but not connected to another strut or intersection.

Struts 16 and intersections 18 can be considered to define the shape and configuration of the pores 20 that make up void phase 14 (or vice versa). Many of pores 20, in so far as they may be discretely identified, open into and communicate, by the at least partial absence of cell walls 22, with at least two other pores 20. At intersections 18, three or more pores 20 may be considered to meet and intercommunicate. In certain embodiments, void phase 14 is continuous or substantially continuous throughout elastomeric matrix 10, meaning that there are few if any closed cell In another embodiment, closed cell pores, if present, comprise less than about 60% of the volume of elastomeric matrix 10. In another embodiment, closed cell pores, if present, comprise less than about 50% of the volume of elastomeric matrix 10. In another embodiment, closed cell pores, if present, comprise less than about 30% of the volume of elastomeric matrix 10. In another embodiment, closed cell pores, if present, comprise less than about 25% of the volume of elastomeric matrix 10. In another embodiment, closed cell pores, if present, comprise less than about 20% of the volume of elastomeric matrix 10. In another embodiment, closed cell pores, if present, comprise less than about 15% of the volume of elastomeric matrix 10. In another embodiment, closed cell pores, if present, comprise less than about 10% of the volume of elastomeric matrix 10. In another embodiment, closed cell pores, if present, comprise less than about 5% of the volume of elastomeric matrix 10. In another embodiment, closed cell pores, if present, comprise less than about 2% of the volume of elastomeric matrix 10. The presence of closed cell pores can be noted by their influence in reducing the volumetric flow rate of a fluid through elastomeric matrix 10 and/or as a reduction in cellular ingrowth and proliferation into elastomeric matrix 10.

In another embodiment, elastomeric matrix 10 is reticulated. In another embodiment, elastomeric matrix 10 is substantially reticulated. In another embodiment, elastomeric matrix 10 is fully reticulated. In another embodiment, elastomeric matrix 10 has many cell walls 22 removed. In another embodiment, elastomeric matrix 10 has most cell walls 22 removed. In another embodiment, elastomeric matrix 10 has substantially all cell walls 22 removed.

In another embodiment, void phase 14 is also a continuous network of interstitial spaces, or intercommunicating fluid passageways for gases or liquids, which fluid passageways extend throughout and are defined by (or define) the structure of solid phase 12 of elastomeric matrix 10 and open into all its exterior surfaces. In another embodiment, void phase 14 of elastomeric matrix 10 is continuous and fully accessible and interconnected and inter-communicating. In another embodiment, void phase 14 of elastomeric matrix 10 is a continuous interconnected and inter-communicating network of voids, cells and pores and this continuous void phase is the principle characteristic of the reticulated matrix. In other embodiments, as described above, there are only a few, substantially no, or no occlusions or closed cell pores that do not communicate with at least one other pore 20 in the void network. Also in this void phase network, a hypothetical line may be traced entirely through void phase 14 from one point in the network to any other point in the network.

In concert with the objectives of the invention, in one embodiment the microstructure of elastomeric matrix 10 is constructed to permit or encourage cellular adhesion to the surfaces of solid phase 12, neointima formation thereon and cellular and tissue ingrowth and proliferation into pores 20 of void phase 14, when elastomeric matrix 10 resides in suitable in vivo locations for a period of time.

In another embodiment, such cellular or tissue ingrowth and proliferation, which may for some purposes include fibrosis, can occur or be encouraged not just into exterior layers of pores 20, but into the deepest interior of and throughout elastomeric matrix 10. This is possible owing to the presence of interconnected and inter-communicating cells and pores and voids, all of which are accesible for cellular or tissue ingrowth and proliferation. Thus, in this embodiment, the space occupied by elastomeric matrix 10 becomes entirely filled by the cellular and tissue ingrowth and proliferation in the form of fibrotic, scar or other tissue except for the space occupied by the elastomeric solid phase 12.

To this end, particularly with regard to the morphology of void phase 14, in one embodiment elastomeric matrix 10 is reticulated with open interconnected and inter-communicating pores. Without being bound by any particular theory, this is thought to permit natural irrigation of the interior of elastomeric matrix 10 with bodily fluids, e.g., blood, even after a cellular population has become resident in the interior of elastomeric matrix 10 so as to sustain that population by supplying nutrients thereto and removing waste products therefrom. In another embodiment, elastomeric matrix 10 is reticulated with open interconnected and inter-communicating pores of a particular size range. In another embodiment, elastomeric matrix 10 is reticulated with open interconnected and inter-communicating pores pores with a distribution of size ranges. In another embodiment, elastomeric matrix 10 is reticulated with interconnected and inter-communicating cell and pores and voids, all of which are accesible by bodily fluids and cells and tissues.

It is intended that the various physical and chemical parameters of elastomeric matrix 10 including in particular the parameters to be described below, be selected to encourage cellular ingrowth and proliferation also tissue ingrowth and proliferation according to the particular application for which an elastomeric matrix 10 is intended.

It will be understood that such constructions of elastomeric matrix 10 that provide interior cellular irrigation will be fluid permeable and may also provide fluid access through and to the interior of the matrix for purposes other than cellular irrigation, for example, for elution of pharmaceutically-active agents, e.g., a drug, or other biologically useful materials. Such materials may optionally be secured to the interior surfaces of elastomeric matrix 10.

In another embodiment of the invention, gaseous phase 12 can be filled or contacted with a deliverable treatment gas, for example, a sterilant such as ozone or a wound healant such as nitric oxide, provided that the macrostructural surfaces are sealed, for example by a bioabsorbable membrane to contain the gas within the implanted product until the membrane erodes releasing the gas to provide a local therapeutic or other effect.

Porosity

Post-reticulation, void phase 14 may comprise as little as 10% by volume of elastomeric matrix 10, referring to the volume provided by the interstitial spaces of elastomeric matrix 10 before any optional interior pore surface coating or layering is applied, such as for a reticulated elastomeric matrix that, after reticulation, has been compressively molded and/or reinforced as described in detail herein. In another embodiment, void phase 14 may comprise as little as 20% by volume of elastomeric matrix 10. In another embodiment, void phase 14 may comprise as little as 35% by volume of elastomeric matrix 10. In another embodiment, void phase 14 may comprise as little as 50% by volume of elastomeric matrix 10. In one embodiment, the volume of void phase 14, as just defined, is from about 10% to about 99% of the volume of elastomeric matrix 10. In another embodiment, the volume of void phase 14, as just defined, is from about 20% to about 99% of the volume of elastomeric matrix 10. In another embodiment, the volume of void phase 14, as just defined, is from about 30% to about 97% of the volume of elastomeric matrix 10. In another embodiment, the volume of void phase 14, as just defined, is from about 50% to about 99% of the volume of elastomeric matrix 10. In another embodiment, the volume of void phase 14, as just defined, is from about 70% to about 99% of the volume of elastomeric matrix 10. In another embodiment, the volume of void phase 14 is from about 80% to about 98% of the volume of elastomeric matrix 10. In another embodiment, the volume of void phase 14 is from about 90% to about 98% of the volume of elastomeric matrix 10. In another embodiment, the volume of void phase 14 is from about 90% to about 99% of the volume of elastomeric matrix 10. In another embodiment, the volume of void phase 14 is from about 95% to about 99% of the volume of elastomeric matrix 10. In another embodiment, the volume of void phase 14 is from about 96% to about 99% of the volume of elastomeric matrix 10.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. As used herein, the “average diameter or other largest transverse dimension” refers to the number average diameter, for spherical or substantially spherical pores, or to the number average largest transverse dimension, for non-spherical pores.

In one embodiment relating to orthopedic applications, hernia applications, surgical mesh applications and the like, to encourage cellular ingrowth and proliferation and to provide adequate fluid permeability, the average diameter or other largest transverse dimension of pores 20 is at least about 10 μm. In another embodiment, the average diameter or other largest transverse dimension of pores 20 is at least about 20 μm. In another embodiment, the average diameter or other largest transverse dimension of pores 20 is at least about 50 μm. In another embodiment, the average diameter or other largest transverse dimension of pores 20 is at least about 100 μm. In another embodiment, the average diameter or other largest transverse dimension of pores 20 is at least about 150 μm. In another embodiment, the average diameter or other largest transverse dimension of pores 20 is at least about 250 μm. In another embodiment, the average diameter or other largest transverse dimension of pores 20 is greater than about 250 μm. In another embodiment, the average diameter or other largest transverse dimension of pores 20 is greater than 250 μm. In another embodiment, the average diameter or other largest transverse dimension of pores 20 is at least about 450 μm. In another embodiment, the average diameter or other largest transverse dimension of pores 20 is greater than about 450 μm. In another embodiment, the average diameter or other largest transverse dimension of pores 20 is greater than 450 μm. In another embodiment, the average diameter or other largest transverse dimension of pores 20 is at least about 500 μm.

In another embodiment relating to soft tissue such as orthopedic applications, hernia applications, surgical mesh applications and the like, the average diameter or other largest transverse dimension of pores 20 is not greater than about 600 μm. In another embodiment, the average diameter or other largest transverse dimension of pores 20 is not greater than about 500 μm. In another embodiment, the average diameter or other largest transverse dimension of pores 20 is not greater than about 450 μm. In another embodiment, the average diameter or other largest transverse dimension of pores 20 is not greater than about 350 μm. In another embodiment, the average diameter or other largest transverse dimension of pores 20 is not greater than about 250 μm. In another embodiment, the average diameter or other largest transverse dimension of pores 20 is not greater than about 150 μm. In another embodiment, the average diameter or other largest transverse dimension of pores 20 is not greater than about 20 μm.

In another embodiment relating to orthopedic applications, hernia applications, surgical mesh applications and the like, the average diameter or other largest transverse dimension of the cells of elastomeric matrix 10 is not greater than about 1000 μm. In another embodiment, the average diameter or other largest transverse dimension of its cells is not greater than about 850 μm. In another embodiment, the average diameter or other largest transverse dimension of its cells is not greater than about 450 μm. In another embodiment, the average diameter or other largest transverse dimension of its cells is not greater than about 700 μm. In another embodiment, the average diameter or other largest transverse dimension of its cells is not greater than about 650 μm. In another embodiment, the average diameter or other largest transverse dimension of its cells is not greater than about 900 μm. In another embodiment, the average diameter or other largest transverse dimension of its cells is not greater than about 1200 μm.

In another embodiment relating to orthopedic applications, hernia applications, surgical mesh applications and the like, the average diameter or other largest transverse dimension of the cells of elastomeric matrix 10 is from about 100 μm to about 1000 μm. In another embodiment, the average diameter or other largest transverse dimension of its cells is from about 150 μm to about 850 μm. In another embodiment, the average diameter or other largest transverse dimension of its cells is from about 150 μm to about 1200 μm. In another embodiment, the average diameter or other largest transverse dimension of its cells is from about 200 μm to about 700 μm. In another embodiment, the average diameter or other largest transverse dimension of its cells is from about 250 μm to about 650 μm.

It is well known that hunam or animal cells will adhere, proliferate and differentiate along and through the contours of the structure formed by the pore size distribution. The cell orientation and cell morphology will result in engineered or newly-formed tissue that may substantially replicate or mimic the anatomical features of real tissues, e.g., of the tissues being replaced. This preferential cell morphology and orientation ascribed to the continuous or step-wise pore size distribution variations, with or without pore orientation, can occur when the implantable device is placed, without prior cell seeding, into the tissue repair and regeneration site. This preferential cell morphology and orientation ascribed to the continuous or step-wise pore size distribution can also occur when the implantable device is placed into a patient, e.g., human or animal, tissue repair and regeneration site after being subjected to in vitro cell culturing. These continuous or step-wise pore size distribution variations, with or without pore orientation, can be important characteristics for TE scaffolds in a number of orthopedic applications, especially in soft tissue attachment, repair, regeneration, augmentation and/or support encompassing the spine, shoulder, knee, hand or joints, and in the growth of a prosthetic organ. In another embodiment, these continuous or step-wise pore size distribution variations, with or without pore orientation, can be important characteristics for TE scaffolds in a number of repair and regenertaion of soft tissue defects such as number of hernia applications and is the use of surgical meshes for regeneration, augmentation, etc. These continuous or step-wise pore size distribution variations, with or without pore orientation, can be important characteristics for TE scaffolds in a number of repair of soft tissue defects, specifically inguinal, femoral, ventral, incisional, umbilical, and epigastric hernias.

Size and Shape

Elastomeric matrix 10 or composite mesh comprising reticulated elastomeric matrix 10 can be readily fabricated in any desired size and shape. It is a benefit of the invention that elastomeric matrix 10 is suitable for mass production from bulk stock by subdividing such bulk stock, e.g., by cutting, machining, die punching, laser slicing, or compression molding. In one embodiment, subdividing the bulk stock can be done using a heated surface. It is a further benefit of the invention that the shape and configuration of elastomeric matrix 10 may vary widely and can readily be adapted to desired anatomical morphologies.

The size, shape, configuration and other related details of elastomeric matrix 10 can be either customized to a particular application or patient or standardized for mass production. However, economic considerations may favor standardization. To this end, elastomeric matrix 10 or reticulated elastomeric matrix 10 or composite mesh comprising reticulated elastomeric matrix 10 can be embodied in a kit comprising elastomeric implantable device pieces of different sizes and shapes. Also, as discussed elsewhere in the present specification and as is disclosed in the applications to which priority is claimed, multiple, e.g. two, three or four, individual elastomeric matrices 10 or or composite mesh comprising reticulated elastomeric matrix 10 can be used as an implantable device system for a single target biological site, being sized or shaped or both sized and shaped to function cooperatively for treatment of an individual target site.

The practitioner performing the procedure, who may be a surgeon or other medical or veterinary practitioner, researcher or the like, may then choose one or more implantable devices from the available range to use for a specific treatment, for example, as is described in the applications to which priority is claimed.

By way of example, the minimum dimension of elastomeric matrix 10 or composite mesh comprising reticulated elastomeric matrix 10 may be as little as 0.5 mm and the maximum dimension as much as 100 mm or even greater. In another embodiment, the minimum dimension of elastomeric matrix 10 or composite mesh comprising reticulated elastomeric matrix 10 may be as little as 0.5 mm and the maximum dimension as much as 200 mm or even greater. However, in one embodiment it is contemplated that an elastomeric matrix 10 or composite mesh comprising reticulated elastomeric matrix 10 of such dimension intended for implantation would have an elongated shape, such as the shapes of cylinders, rods, tubes or elongated prismatic forms, or a folded, coiled, helical or other more compact configuration. In another embodiment, it is contemplated that an elastomeric matrix 10 or composite mesh comprising reticulated elastomeric matrix 10 of such dimension intended for implantation would have a shape of a flat sheet or a long ribbon or a folded sheet with square or rectangular configuration. Comparably, a dimension as small as 0.5 mm can be a transverse dimension or the cross-sectional dimension of an elongated shape or of a ribbon or sheet-like implantable device.

In an alternative embodiment, an elastomeric matrix 10 or composite mesh comprising reticulated elastomeric matrix 10 having a spherical, cubical, tetrahedral, toroidal or other form having no dimension substantially elongated when compared to any other dimension and with a diameter or other maximum dimension of from about 0.5 mm to about 500 mm may have utility, for example, for an orthopedic application site, soft tissue defect site such as various forms of hernias, other soft tissue defect site for augmentation, support and ingrowth that require surgical meshes and wound healing sites. In another embodiment, the elastomeric matrix 10 or composite mesh comprising reticulated elastomeric matrix 10 having such a form has a diameter or other maximum dimension from about 3 mm to about 20 mm. In another embodiment, the elastomeric matrix 10 having such a form has a diameter or other maximum dimension from about 0.7 mm to about 300 mm.

For treatment of orthopedic applications, hernia applications, surgical mesh appplications for augmentation, support and ingrowth, it is an advantage of the invention that the implantable elastomeric matrix elements or composite mesh comprising reticulated elastomeric matrix 10 can be effectively employed without any need to closely conform to the configuration of the orthopedic application site, which may often be complex and difficult to model. Thus, in one embodiment, the implantable elastomeric matrix elements of the invention have significantly different and simpler configurations, for example, as described in the applications to which priority is claimed. Another advantage of the invention is that the implantable elastomeric matrix elements or composite mesh comprising reticulated elastomeric matrix 10 embodiment is that when oversized with respect to the soft tisue defect which can be for orthopedic or hernia repair, the implantable device conformally fits the tissue defect. Without being bound by any particular theory, the resilience and recoverable behavior that leads to such a conformal fit results in the formation of a tight boundary between the walls of the implantable device and the defect with substantially no clearance, thereby providing an interface conducive to the promotion of cellular ingrowth and tissue proliferation.

Furthermore, in one embodiment, the implantable device of the present invention, or implantable devices if more than one is used, should not completely fill the application site even when fully expanded in situ. The application site can be orthopedic application site, soft tissue defect site such as various forms of hernias, other soft tissue defect site for augmentation, support and ingrowth that require surgical meshes and wound healing sites. In one embodiment, the fully expanded implantable device(s) of the present invention are smaller in a dimension than the application site and provide sufficient space within the application site to ensure vascularization, cellular ingrowth and proliferation, and for possible passage of blood to the implantable device. In another embodiment, the fully expanded implantable device(s) of the present invention are substantially the same in a dimension as the application site. In another embodiment, the fully expanded implantable device(s) of the present invention are larger in a dimension than the application site. In another embodiment, the fully expanded implantable device(s) of the present invention are smaller in volume than the orthopedic application site. In another embodiment, the fully expanded implantable device(s) of the present invention are substantially the same volume as application site. In another embodiment, the fully expanded implantable device(s) of the present invention are larger in volume than the application site.

In another embodiment, after being placed in the application site the expanded implantable device(s) of the present invention does not swell signifiantly or appreciably. The reticulated matrix or the implantable device(s) of the present invention are not considered to be an expansible material or a hydrogel or water swellable. The reticulated matrix is not considered to be a foam gel. The reticulated matrix does not expand swell on contact with bodily fluid or blood or water. In one embodiment, the reticulated matrix does not substantially expand or swell on contact with bodily fluid or blood or water.

It is contemplated, in another embodiment, that upon implantation, before their pores become filled with biological fluids, bodily fluids and/or tissue, such implantable devices for applications such as soft tissue orthopedic defect, soft tissue defect site such as various forms of hernias, other soft tissue defect site for augmentation, support and ingrowth that require surgical meshes and wound healing sites do not entirely fill, cover or span the biological site in which they reside and that an individual implanted elastomeric matrix 10 or composite mesh comprising reticulated elastomeric matrix 10 will, in many cases although not necessarily, have at least one dimension of no more than 50% of the biological site within the entrance thereto or over 50% of the damaged tissue that is being repaired or replaced. In another embodiment, an individual implanted elastomeric matrix 10 as described above or composite mesh comprising reticulated elastomeric matrix 10 will have at least one dimension of no more than 75% of the biological site within the entrance thereto or over 75% of the damaged tissue that is being repaired or replaced. In another embodiment, an individual implanted elastomeric matrix 10 as described above or composite mesh comprising reticulated elastomeric matrix 10 will have at least one dimension of no more than 95% of the biological site within the entrance thereto or over 95% of the damaged tissue that is being repaired or replaced.

In another embodiment, that upon implantation, before their pores become filled with biological fluids, bodily fluids and/or tissue, such implantable devices for applications such as soft tissue orthopedic defect, soft tissue defect site such as various forms of hernias, other soft tissue defect site for augmentation, support and ingrowth that require surgical meshes and wound healing sites substantially fill, cover or span the biological site in which they reside and an individual implanted elastomeric matrix 10 or composite mesh comprising reticulated elastomeric matrix 10 will, in many cases, although not necessarily, have at least one dimension of no more than about 100% of the biological site within the entrance thereto or cover 100% of the damaged tissue that is being repaired or replaced. In another embodiment, an individual implanted elastomeric matrix 10 as described above or composite mesh comprising reticulated elastomeric matrix 10 will have at least one dimension of no more than about 98% of the biological site within the entrance thereto or cover 98% of the damaged tissue that is being repaired or replaced. In another embodiment, an individual implanted elastomeric matrix 10 as described or composite mesh comprising reticulated elastomeric matrix 10 above will have at least one dimension of no more than about 102% of the biological site within the entrance thereto or cover 102% of the damaged tissue that is being repaired or replaced.

In another embodiment, that upon implantation, before their pores become filled with biological fluids, bodily fluids and/or tissue, such implantable devices for applications such as soft tissue orthopedic defect, soft tissue defect site such as various forms of hernias, other soft tissue defect site for augmentation, support and ingrowth that require surgical meshes and wound healing sites over fill, cover or span the biological site in which they reside and an individual implanted elastomeric matrix 10 or composite mesh comprising reticulated elastomeric matrix 10 will, in many cases, although not necessarily, have at least one dimension of more than about 105% of the biological site within the entrance thereto or cover 105% of the damaged tissue that is being repaired or replaced. In another embodiment, an individual implanted elastomeric matrix 10 as described above or composite mesh comprising reticulated elastomeric matrix 10 will have at least one dimension of more than about 125% of the biological site within the entrance thereto or cover 125% of the damaged tissue that is being repaired or replaced. In another embodiment, an individual implanted elastomeric matrix 10 as described above or composite mesh comprising reticulated elastomeric matrix 10 will have at least one dimension of more than about 150% of the biological site within the entrance thereto or cover 150% of the damaged tissue that is being repaired or replaced. In another embodiment, an individual implanted elastomeric matrix 10 as described or composite mesh comprising reticulated elastomeric matrix 10 above will have at least one dimension of more than about 200% of the biological site within the entrance thereto or cover 200% of the damaged tissue that is being repaired or replaced. In another embodiment, an individual implanted elastomeric matrix 10 as described or composite mesh comprising reticulated elastomeric matrix 10 above will have at least one dimension of more than about 300% of the biological site within the entrance thereto or cover 300% of the damaged tissue that is being repaired or replaced.

One embodiment for use in the practice of the invention is a reticulated elastomeric matrix 10 which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration in situ. Furthermore, in another embodiment, an elastomeric matrix has the herein described resilient-compressibility after being compressed about 5-95% of an original dimension (e.g., compressed about 19/20th- 1/20th of an original dimension). In another embodiment, an elastomeric matrix has the herein described resilient-compressibility after being compressed about 10-90% of an original dimension (e.g., compressed about 9/10th- 1/10th of an original dimension). As used herein, elastomeric matrix 10 has “resilient-compressibility”, i.e., is “resiliently-compressible”, when the second, working configuration, in vitro, is at least about 50% of the size of the relaxed configuration in at least one dimension. In another embodiment, the resilient-compressibility of elastomeric matrix 10 is such that the second, working configuration, in vitro, is at least about 80% of the size of the relaxed configuration in at least one dimension. In another embodiment, the resilient-compressibility of elastomeric matrix 10 is such that the second, working configuration, in vitro, is at least about 90% of the size of the relaxed configuration in at least one dimension. In another embodiment, the resilient-compressibility of elastomeric matrix 10 is such that the second, working configuration, in vitro, is at least about 97% of the size of the relaxed configuration in at least one dimension.

One embodiment for use in the practice of the invention is a r composite mesh comprising reticulated elastomeric matrix which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration in situ.

Elastomeric Matrix Physical Properties

Elastomeric matrix 10, a reticulated elastomeric matrix, an implantable device comprising a reticulated elastomeric matrix, and/or an implantable device comprising a compressive molded reticulated elastomeric matrix can have any suitable bulk density, also known as specific gravity, consistent with its other properties. For example, in one embodiment, the bulk density, as measured pursuant to the test method described in ASTM Standard D3574, may be from about 0.005 g/cc to about 0.96 g/cc (from about 0.31 lb/ft³ to about 60 lb/ft³). In another embodiment, the bulk density may be from about 0.048 g/cc to about 0.56 g/cc (from about 3.0 lb/ft³ to about 35 lb/ft³). In another embodiment, the bulk density may be from about 0.005 g/cc to about 0.15 g/cc (from about 0.31 lb/ft³ to about 9.4 lb/ft³). In another embodiment, the bulk density may be from about 0.008 g/cc to about 0.127 g/cc (from about 0.5 lb/ft³ to about 8 lb/ft³). In another embodiment, the bulk density may be from about 0.015 g/cc to about 0.115 g/cc (from about 0.93 lb/ft³ to about 7.2 lb/ft³). In another embodiment, the bulk density may be from about 0.024 g/cc to about 0.104 g/cc (from about 1.5 lb/ft³ to about 6.5 lb/ft³).

In one embodiment, reticulated elastomeric matrix 10 has sufficient structural integrity to be self-supporting and free-standing in vitro. However, in another embodiment, elastomeric matrix 10 can be furnished with structural supports such as ribs or struts.

The reticulated elastomeric matrix 10 has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. Thus, for example, in one embodiment reticulated elastomeric matrix 10 may have a tensile strength of from about 700 kg/m² to about 350,000 kg/m² (from about 1 psi to about 500 psi). In another embodiment, elastomeric matrix 10 may have a tensile strength of from about 700 kg/m² to about 70,000 kg/m² (from about 1 psi to about 100 psi). In another embodiment, elastomeric matrix 10 may have a tensile strength of from about 3,500 to about 140,000 kg/m² (from about 5 to about 200 psi). In another embodiment, elastomeric matrix may have a tensile strength of from about 14,000 to about 105,000 kg/m² (from about 20 to about 150 psi). In another embodiment, reticulated elastomeric matrix 10 may have a tensile modulus of from about 1,400 kg/m² to about 140,000 kg/m² (from about 2 psi to about 200 psi). In another embodiment, reticulated elastomeric matrix 10 may have a tensile modulus of from about 3,500 kg/m² to about 105,000 kg/m² (from about 5 psi to about 150 psi). In another embodiment, elastomeric matrix 10 may have a tensile modulus of from about 17,500 kg/m² to about 70,000 kg/m² (from about 25 psi to about 100 psi).

Sufficient ultimate tensile elongation is also desirable. For example, in another embodiment, reticulated elastomeric matrix 10 has an ultimate tensile elongation of at least about 25%. In another embodiment, elastomeric matrix 10 has an ultimate tensile elongation of at least about 50%. In another embodiment, elastomeric matrix 10 has an ultimate tensile elongation of at least about 75%. In another embodiment, elastomeric matrix 10 has an ultimate tensile elongation of at least about 150%. In another embodiment, elastomeric matrix 10 has an ultimate tensile elongation of at least about 50% to at least about 400%. In another embodiment, reticulated elastomeric matrix 10 has an ultimate tensile elongation of at least 75% to at least about 300%. In yet another embodiment, reticulated elastomeric matrix 10 has an ultimate tensile elongation of at least about 100% to at least about 250%.

In one embodiment, the elastomeric matrix 10 expands from the first, compact configuration to the second, working configuration over a short time, e.g., about 95% recovery in 90 seconds or less in one embodiment, or in 40 seconds or less in another embodiment, each from 75% compression strain held for up to 10 minutes. In another embodiment, the expansion from the first, compact configuration to the second, working configuration occurs over a short time, e.g., about 95% recovery in 180 seconds or less in one embodiment, in 90 seconds or less in another embodiment, in 60 seconds or less in another embodiment, each from 75% compression strain held for up to 30 minutes. In another embodiment, elastomeric matrix 10 recovers in about 10 minutes to occupy at least about 97% of the volume occupied by its relaxed configuration, following 75% compression strain held for up to 30 minutes. In another embodiment, elastomeric matrix 10 recovers in about 10 minutes to occupy at least about 97% of the volume occupied by its relaxed configuration, following 75% compression strain held for up to 30 minutes.

In one embodiment, reticulated elastomeric matrix 10 may have a compressive modulus of from about 1,400 kg/m² to about 140,000 kg/m² (from about 2 psi to about 200 psi). In another embodiment, reticulated elastomeric matrix 10 may have a compressive modulus of from about 3,500 kg/m² to about 105,000 kg/m² (from about 5 psi to about 150 psi). In another embodiment, elastomeric matrix 10 may have a compressive modulus of from about 17,500 kg/m² to about 70,000 kg/m² (from about 25 psi to about 100 psi).

In another embodiment, reticulated elastomeric matrix 10 has a compressive strength of from about 210 kg/m² to about 35,000 kg/m² (from about 0.3 psi to about 50 psi) at 50% compression strain. In another embodiment, reticulated elastomeric matrix 10 has a compressive strength of from about 350 kg/m² to about 10,500 kg/m² (from about 0.5 psi to about 15 psi) at 50% compression strain. In another embodiment, reticulated elastomeric matrix 10 has a compressive strength of form about 490 kg/m² to about 70,000 kg/m² (from about 0.7 psi to about 100 psi) at 75% compression strain. In another embodiment, reticulated elastomeric matrix 10 has a compressive strength of from about 560 kg/m² to about 24,500 kg/m² (from about 0.8 psi to about 35 psi) at 75% compression strain.

In another embodiment, reticulated elastomeric matrix 10 has a compression set, when compressed to 50% of its thickness at about 25° C., i.e., pursuant to ASTM D3574, of not more than about 30%. In another embodiment, elastomeric matrix 10 has a compression set of not more than about 20%. In another embodiment, elastomeric matrix 10 has a compression set of not more than about 10%. In another embodiment, elastomeric matrix 10 has a compression set of not more than about 5%.

In another embodiment, reticulated elastomeric matrix 10 has a tear strength, as measured pursuant to the test method described in ASTM Standard D3574, of from about 0.18 kg/linear cm to about 8.90 kg/linear cm (from about 1 lbs/linear inch to about 50 lbs/linear inch). In another embodiment, reticulated elastomeric matrix 10 has a tear strength, as measured pursuant to the test method described in ASTM Standard D3574, of from about 0.18 kg/linear cm to about 1.78 kg/linear cm (from about 1 lbs/linear inch to about 10 lbs/linear inch).

In another embodiment, reticulated elastomeric matrix 10 has a static recovery time, t-90% (as measured by the time to recover the 90% of the original thickness after the reticulated elastomeric matrix 10 was subject to 50% strain over 120 minutes) was of from about 10 sec. to about 1000 sec. In another embodiment, reticulated elastomeric matrix 10 has a static recovery time, t-90%, of from about 20 sec. to about 500 sec. In another embodiment, reticulated elastomeric matrix 10 has a static recovery time, t-90%, of from about 25 sec. to about 200 sec.

Biodurability and Biocompatibility

In one embodiment, elastomers are sufficiently biodurable so as to be suitable for long-term implantation in patients, e.g., animals or humans. Biodurable elastomers and elastomeric matrices have chemical, physical and/or biological properties so as to provide a reasonable expectation of biodurability, meaning that the elastomers will continue to exhibit stability when implanted in an animal, e.g., a mammal, for a period of at least 29 days. The intended period of long-term implantation may vary according to the particular application. For many applications, substantially longer periods of implantation may be required and for such applications biodurability for periods of at least 6, 12 or 24 months or 5 years, or longer, may be desirable. Of especial benefit are elastomers that may be considered biodurable for the life of a patient. In the case of the possible use of an embodiment of elastomeric matrix 10 to treat such conditions may present themselves in rather young human patients, perhaps in their thirties, biodurability in excess of 50 years may be advantageous.

Without being bound by any particular theory, biodurability of the elastomeric matrix formed by a process comprising polymerization, cross-linking, foaming and reticulation and include the selection of starting components that are biodurable and the stoichiometric ratios of those components, such that the elastomeric matrix retains the biodurability of its components. Further following reticulation, more extensive washing in exemplery solvents such as isopropyl alcohol are used to remove unreacted chemical ingredients or processing aids from the reticulated matrix. For example, elastomeric matrix biodurability can be promoted by minimizing or eliminating the presence and formation of chemical bonds and groups, such as ester groups, that are susceptible to hydrolysis, e.g., at the patient's body fluid temperature and pH. In another example, elastomeric matrix biodurability can be promoted by minimizing or eliminating the presence and formation of chemical bonds and groups, such as polyether groups, that are susceptible to oxidative degradation , e.g., at the patient's body fluid temperature and pH or by action of enzymes and cells in the body. As a further example, a curing step in excess of about 2 hours can be performed after cross-linking and foaming to minimize the presence of free amine groups in the elastomeric matrix. Moreover, it is important to minimize degradation that can occur during the elastomeric matrix preparation process, e.g., because of exposure to shearing or thermal energy such as may occur during admixing, dissolution, cross-linking and/or foaming, by ways known to those in the art. Without being bound by any particular theory, biodurability of the elastomeric matrix is also enahnced by the chemical and physical cross-linkings that are present in elastomeric matrix 10.

As previously discussed, biodurable elastomers and elastomeric matrices are stable for extended periods of time in a biological environment. Such products do not exhibit significant symptoms of breakdown, degradation, erosion or significant deterioration of mechanical properties relevant to their use when exposed to biological environments and/or bodily stresses for periods of time commensurate with that use. Furthermore, in certain implantation applications, it is anticipated that elastomeric matrix 10 will become in the course of time, for example, in 2 weeks to 1 year, will promote cellular ingrowth followed by ingrowth and proliferation of tissues that will remodel over time or incorporated and totally integrated or bio-integrated into, e.g., the tissue being repaired or the lumen being treated. In this condition, elastomeric matrix 10 has reduced exposure to mobile or circulating biological fluids. Accordingly, the probabilities of biochemical degradation or release of undesired, possibly nocuous, products into the host organism may be attenuated if not eliminated. Owing to the reticulated nature of the elastomeric matrix 10 that comprises of interconnected and inter-communicating network of cell pore and voids that allow for easy passage of body fluids and tissues, the possibility of elastomeric matrix 10 being walled-off or becoming encapsulated by tissue is unlikely. The reticulated nature of elastomeric matrix 10 is believed to limit the undesirable fibrotic scarring and limit undesirable encapsulation as has been observed from the results of the in vivio implantation studies.

Elastomeric Matrices from Elastomer Polymerization, Cross-Linking and Foaming

In further embodiments, the invention provides a porous biodurable elastomer and a process for polymerizing, cross-linking and foaming the same which can be used to produce a biodurable reticulated elastomeric matrix 10 as described herein. In another embodiment, reticulation follows.

More particularly, in another embodiment, the invention provides a process for preparing a biodurable elastomeric polyurethane matrix which comprises synthesizing the matrix from a polycarbonate polyol component and an isocyanate component by polymerization, cross-linking and foaming, thereby forming pores, followed by reticulation of the foam to provide a reticulated product. The product is designated as a polycarbonate polyurethane, being a polymer comprising urethane groups formed from, e.g., the hydroxyl groups of the polycarbonate polyol component and the isocyanate groups of the isocyanate component. In this embodiment, the process employs controlled chemistry to provide a reticulated elastomer product with good biodurability characteristics. Pursuant to the invention, the polymerization is conducted to provide a foam product employing chemistry that avoids biologically undesirable or nocuous constituents therein.

In one embodiment, as one starting material, the process employs at least one polyol component. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. Exemplary polyols can comprise, on the average, from about 2 to about 5 hydroxyl groups per molecule. In one embodiment, as one starting material, the process employs a difunctional polyol component. In this embodiment, because the hydroxyl group functionality of the diol is about 2, it does not provide the so-called “soft segment” with soft segment cross-linking another embodiment, the soft segment is composed of a polyol component that is generally of a relatively low molecular weight, in one embodiment from about 350 to about 6,000 Daltons, and from about 450 to about 4,000 Daltons in another embodiment. Thus, these polyols are generally liquids or low-melting-point solids.

Polycarbonate-type polyols typically result from the reaction, with a carbonate monomer, of one type of hydrocarbon diol or, for a plurality of diols, hydrocarbon diols each with a different hydrocarbon chain length between the hydroxyl groups The molecular weight for the commercial-available products of this reaction varies from about 500 to about 5,000 Daltons. If the polycarbonate polyol is a solid at 25° C., it is typically melted prior to further processing.

Polysiloxane polyols are oligomers of, e.g., alkyl and/or aryl substituted siloxanes such as dimethyl siloxane, diphenyl siloxane or methyl phenyl siloxane, comprising hydroxyl end-groups. Polysiloxane polyols with an average number of hydroxyl groups per molecule greater than 2, e.g., a polysiloxane triol, can be made by using, for example, methyl hydroxymethyl siloxane, in the preparation of the polysiloxane polyol component.

Additionally, in another embodiment, copolymers or copolyols can be formed from any of the above polyols by methods known to those in the art In another embodiment, the polyol component is a polycarbonate polyol, hydrocarbon polyol, polysiloxane polyol, poly(carbonate-co-hydrocarbon)polyol, poly(carbonate-co-siloxane)polyol, poly(hydrocarbon-co-siloxane)polyol or a mixture thereof. In another embodiment, the polyol component is a polycarbonate polyol, poly(carbonate-co-hydrocarbon)polyol, poly(carbonate-co-siloxane)polyol, poly(hydrocarbon-co-siloxane)polyolor a mixture thereof. In another embodiment, the polyol component is a polycarbonate polyol, poly(carbonate-co-hydrocarbon)polyol, poly(carbonate-co-siloxane)polyol or a mixture thereof. In another embodiment, the polyol component is a polycarbonate polyol.

Furthermore, in another embodiment, mixtures, admixtures and/or blends of polyols and copolyols can be used in the elastomeric matrix of the present invention. In another embodiment, the molecular weight of the polyol is varied. In another embodiment, the functionality of the polyol is varied.

The process also employs at least one isocyanate component and, optionally, at least one chain extender component to provide the so-called “hard segment”. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, cross-linker and/or water.

In one embodiment, the average number of isocyanate groups per molecule in the isocyanate component is about 2. In another embodiment, the average number of isocyanate groups per molecule in the isocyanate component is greater than about 2. In another embodiment, the average number of isocyanate groups per molecule in the isocyanate component is greater than 2. When the average number of isocyanate groups per molecule in the isocyanate component is greater than 2, it allows for cross-linking to occcu in elastomeric matrix 10. In one embodiment, the cross-linkingis chemical in nature that is formed by covalent bonding. Without being bound by any particular theory, cross-linking adds to biodurability and biostability of the elastomeric matrix 10 and cross-linking also adds to the resiliency and elastomeric nature of elastomeric matrix 10.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98. In another embodiment, the isocyanate index is from about 0.9 to about 1.0. In another embodiment, the isocyanate index is from about 0.9 to about 1.01.

Exemplary diisocyanates include aliphatic diisocyanates, isocyanates comprising aromatic groups, the so-called “aromatic diisocyanates”, or a mixture thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H₁₂ MDI”), or a mixture thereof Aromatic diisocyanates include p-phenylene diisocyanate, 4,4′-diphenylmethane diisocyanate (“4,4′-MDI”), 2,4′-diphenylmethane diisocyanate (“2,4′-MDI”), 2,4-toluene diisocyanate (“2,4-TDI”), 2,6-toluene diisocyanate(“2,6-TDI”), m-tetramethylxylene diisocyanate, or a mixture thereof.

In one embodiment, the isocyanate component contains a mixture of at least about 5% by weight of 2,4′-MDI with the balance 4,4′-MDI. In another embodiment, the isocyanate component contains a mixture of at least 5% by weight of 2,4′-MDI with the balance 4,4′-MDI. In another embodiment, the isocyanate component contains a mixture of from about 5% to about 50% by weight of 2,4′-MDI with the balance 4,4′-MDI. In another embodiment, the isocyanate component contains a mixture of from 5% to about 50% by weight of 2,4′-MDI with the balance 4,4′-MDI. In another embodiment, the isocyanate component contains a mixture of from about 5% to about 40% by weight of 2,4′-MDI with the balance 4,4′-MDI. In another embodiment, the isocyanate component contains a mixture of from 5% to about 40% by weight of 2,4′-MDI with the balance 4,4′-MDI. In another embodiment, the isocyanate component contains a mixture of from 5% to about 35% by weight of 2,4′-MDI with the balance 4,4′-MDI. In another embodiment, the isocyanate component contains a mixture of from about 10% to about 40% by weight of 2,4′-MDI with the balance 4,4′-MDI. In another embodiment, the isocyanate component contains a mixture of from 10% to about 40% by weight of 2,4′-MDI with the balance 4,4′-MDI. In another embodiment, the isocyanate component contains a mixture of from about 20% to about 40% by weight of 2,4′-MDI with the balance 4,4′-MDI. In another embodiment, the isocyanate component contains a mixture of from 20% to about 40% by weight of 2,4′-MDI with the balance 4,4′-MDI. Without being bound by any particular theory, it is thought that the use of higher amounts of 2,4′-MDI in a blend with 4,4′-MDI results in a softer elastomeric matrix because of the disruption of the crystallinity or formation a regular or ordered structure of the hard segment arising out of the asymmetric 2,4′-MDI structure. Without being bound by any particular theory, it is thought that the use of higher amounts of 2,4′-MDI in a blend with 4,4′-MDI results in a softer elastomeric matrix because of the disruption of the more ordered or more organized structure of the hard segment arising out of the asymmetric 2,4′-MDI structure. Higher the amount of the asymmetric 2,4′-MDI lead to more disruption of the crystallinity or formation a regular or ordered structure or more organized in the hard segment.

Exemplary chain extenders include diols, diamines, alkanol amines or a mixture thereof. In one embodiment, the chain extender is an aliphatic diol having from 2 to 10 carbon atoms. In another embodiment, the diol chain extender is selected from ethylene glycol, 1,2-propane diol, 1,3-propane diol, 1,4-butane diol, 1,5-pentane diol, diethylene glycol, triethylene glycol or a mixture thereof. In another embodiemnt, trifunctional or higher chain extenders as cross-linking agents.

In one embodiment, a small quantity of an optional ingredient, such as a multi-functional hydroxyl compound or other cross-linker having a functionality greater than 2, e.g., glycerol, is present to allow cross-linking In one embodiment, the cross-linking is chemical in nature that is formed by covalent bonding. In one embodiment, a small quantity of an optional ingredient, such as a multi-functional amine compound or other cross-linker having a functionality greater than 2 is present to allow cross-linking In another embodiment, the optional multi-functional cross-linker is present in an amount just sufficient to achieve a stable foam, i.e., a foam that does not collapse to become non-foamlike. Alternatively, or in addition, polyfunctional adducts of aliphatic and cycloaliphatic isocyanates can be used to impart cross-linking in combination with aromatic diisocyanates. Alternatively, or in addition, polyfunctional adducts of aliphatic and cycloaliphatic isocyanates can be used to impart cross-linking in combination with aliphatic diisocyanates. When the average number of isocyanate groups per molecule in the isocyanate component is greater than 2, it allows for chemical cross-linking to occcur in elastomeric matrix 10. In another embodiemnt, trifunctional or higher chain extenders as cross-linking agents. Without being bound by any particular theory, cross-linking adds to biodurability and biostability of the elastomeric matrix 10 and cross-linking also adds to the resiliency and elastomeric nature of elastomeric matrix 10.

Optionally, the process employs at least one catalyst in certain embodiments selected from a blowing catalyst, e.g., a tertiary amine, a gelling catalyst, e.g., dibutyltin dilaurate, or a mixture thereof. Moreover, it is known in the art that tertiary amine catalysts can also have gelling effects, that is, they can act as a blowing and gelling catalyst In certain embodiments, the process employs at least one surfactantIn certain embodiments, the process employs at least one cell-opener.

Cross-linked polyurethanes may be prepared by approaches which include the prepolymer process and the one-shot process.

In one embodiment, the invention provides a process for preparing a flexible polyurethane biodurable matrix capable of being reticulated based on polycarbonate polyol component and isocyanate component starting materials. In another embodiment, the foam is substantially free of isocyanurate linkages. In another embodiment, the foam has no isocyanurate linkages. In another embodiment, the foam is substantially free of biuret linkages. In another embodiment, the foam has no biuret linkages. In another embodiment, the foam is substantially free of allophanate linkages. In another embodiment, the foam has no allophanate linkages. In another embodiment, the foam is substantially free of isocyanurate and biuret linkages. In another embodiment, the foam has no isocyanurate and biuret linkages. In another embodiment, the foam is substantially free of isocyanurate and allophanate linkages. In another embodiment, the foam has no isocyanurate and allophanate linkages. In another embodiment, the foam is substantially free of allophanate and biuret linkages. In another embodiment, the foam has no allophanate and biuret linkages. In another embodiment, the foam is substantially free of allophanate, biuret and isocyanurate linkages. In another embodiment, the foam has no allophanate, biuret and isocyanurate linkages. Without being bound by any particular theory, it is thought that the absence of allophanate, biuret and/or isocyanurate linkages provides an enhanced degree of flexibility to the elastomeric matrix because of lower cross-linking of the hard segments.

Exemplary blowing agents include water and the physical blowing agents, e.g., volatile organic chemicals such as hydrocarbons, ethanol and acetone, and various fluorocarbons and their more environmentally friendly replacements, such as hydrofluorocarbons, chlorofluorocarbons and hydrochlorofluorocarbons. The reaction of water with an isocyanate group yields carbon dioxide, which serves as a blowing agent. Moreover, combinations of blowing agents, such as water with a fluorocarbon, can be used in certain embodiments. In another embodiment, water is used as the blowing agent.

In one embodiment, the inventive reticulated biodurable elastomeric matrix are synthetic polymers, especially, but not exclusively, elastomeric polymers that are resistant to biological degradation, for example, polycarbonate polyurethane-urea, polycarbonate polyurea-urethane, polycarbonate polyurethane, polycarbonate polysiloxane polyurethane, and polysiloxane polyurethane, polycarbonate polysiloxane polyurethane urea, polysiloxane polyurethane urea, polycarbonate hydrocarbon polyurethane, polycarbonate hydrocarbon polyurethane urea or any mixture thereof Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic. In another embodiment, such elastomers may be produced with surfaces that are significantly or largely hydrophobic.

Further Process Aspects of the Invention

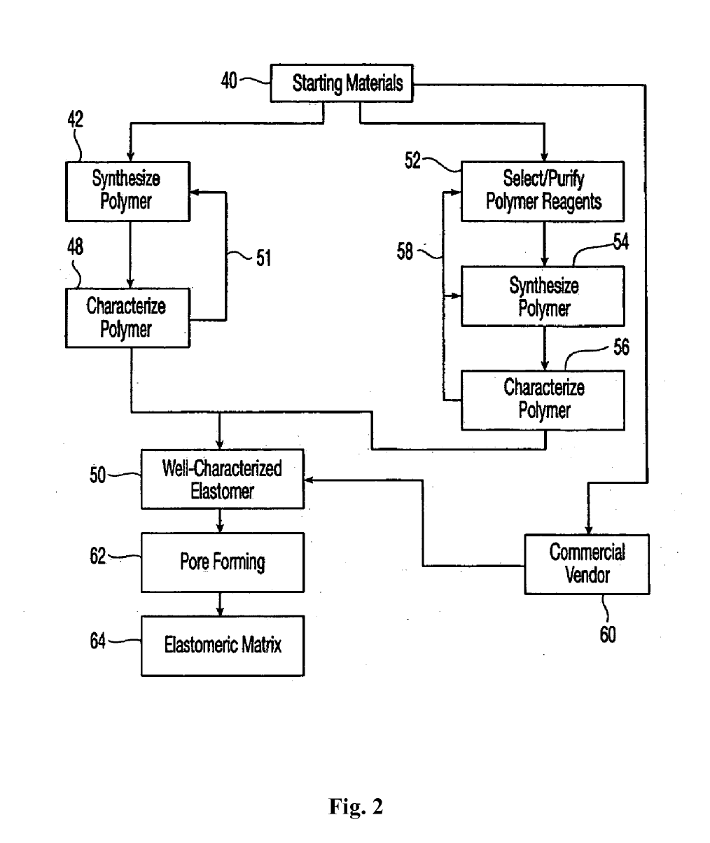

Referring now to FIG. 2, the schematic block flow diagram shown gives a broad overview of alternative embodiments of processes according to the invention whereby an implantable device comprising a biodurable, porous, reticulated, elastomeric matrix 10 can be prepared from raw elastomer or elastomer reagents by one or another of several different process routes.

In a first route, elastomers prepared by a process according to the invention, as described herein, are rendered to comprise a plurality of cells by using, e.g., a blowing agent or agents, employed during their preparation. In particular, starting materials 40, which may comprise, for example, a polyol component, an isocyanate, optionally a cross-linker, and any desired additives such as surfactants and the like, are employed to synthesize the desired elastomeric polymer, in synthesis step 42, either with or without significant foaming or other pore-generating activity. The starting materials are selected to provide desirable mechanical properties and to enhance biocompatibility and biodurability. The elastomeric polymer product of step 42 is then characterized, in step 48, as to chemical nature and purity, physical and mechanical properties and, optionally, also as to biological characteristics, all as described above, yielding well-characterized elastomer 50. Optionally, the characterization data can be employed to control or modify step 42 to enhance the process or the product, as indicated by pathway 51.

Reticulation of Elastomeric Matrices

Elastomeric matrix 10 can be subjected to any of a variety of post-processing treatments to enhance its utility, some of which are described herein and others of which will be apparent to those skilled in the art. In one embodiment, reticulation of an elastomeric matrix 10 of the invention, if not already a part of the described production process, may be used to remove at least a portion of any existing interior “windows”, i.e., the residual cell walls 22 illustrated in FIG. 1. Reticulation tends to increase porosity and fluid permeability.

Porous or foam materials with some ruptured cell walls are generally known as “open-cell” materials or foams. In contrast, porous materials known as “reticulated” or “at least partially reticulated” have many, i.e., at least about 40%, of the cell walls that would be present in an identical porous material except composed exclusively of cells that are closed, at least partially removed. Where the cell walls are least partially removed by reticulation, adjacent reticulated cells open into, interconnect with, and communicate with each other. Porous materials from which more, i.e., at least about 65%, of the cell walls have been removed are known as “further reticulated”. If most, i.e., at least about 80%, or substantially all, i.e., at least about 90%, of the cell walls have been removed then the porous material that remains is known as “substantially reticulated” or “fully reticulated”, respectfully. It will be understood that, pursuant to this art usage, a reticulated material or foam comprises a network of at least partially open interconnected cells.

“Reticulation” generally refers to a process for at least partially removing cell walls, not merely rupturing or tearing them by a crushing process. Moreover, crushing undesirable creates debris that must be removed by further processing. In another embodiment, the reticulation process substantially fully removes at least a portion of the cell walls. Reticulation may be effected, for example, by at least partially dissolving away cell walls, known variously as “solvent reticulation” or “chemical reticulation”; or by at least partially melting, burning and/or exploding out cell walls, known variously as “combustion reticulation”, “thermal reticulation” or “percussive reticulation”. Melted material arising from melted cell walls can be deposited on the struts. In one embodiment, such a procedure may be employed in the processes of the invention to reticulate elastomeric matrix 10. In another embodiment, all entrapped air in the pores of elastomeric matrix 10 is evacuated by application of vacuum prior to reticulation. In another embodiment, reticulation is accomplished through a plurality of reticulation steps. In another embodiment, two reticulation steps are used. In another embodiment, a first combustion reticulation is followed by a second combustion reticulation. In another embodiment, combustion reticulation is followed by chemical reticulation. In another embodiment, chemical reticulation is followed by combustion reticulation. In another embodiment, a first chemical reticulation is followed by a second chemical reticulation.

Optionally, the reticulated elastomeric matrix may be purified, for example, by solvent extraction, either before or after reticulation. Any such solvent extraction, such as with isopropyl alcohol, or other purification process is, in one embodiment, a relatively mild process which is conducted so as to avoid or minimize possible adverse impact on the mechanical or physical properties of the elastomeric matrix that may be necessary to fulfill the objectives of this invention.

One embodiment employs chemical reticulation, where the elastomeric matrix is reticulated in an acid bath comprising an inorganic acid.

In one embodiment, combustion reticulation may be employed in which a combustible atmosphere, e.g., a mixture of hydrogen and oxygen or methane and oxygen, is ignited, e.g., by a spark. In another embodiment, combustion reticulation is conducted in a pressure chamber. In another embodiment, the pressure in the pressure chamber is substantially reduced, e.g., to below about 50-150 torr and preferably below 2-100 torr by evacuation for at least about 2 minutes, before, e.g., hydrogen, oxygen or a mixture thereof, is introduced. In another embodiment, the pressure in the pressure chamber is substantially reduced in more than one cycle, e.g., the pressure is substantially reduced, an unreactive gas such as argon or nitrogen is introduced then the pressure is again substantially reduced, before hydrogen, oxygen or a mixture thereof is introduced. The temperature at which reticulation occurs can be influenced by, e.g., the temperature at which the chamber is maintained and/or by the hydrogen/oxygen ratio in the chamber. In one embodiemnt, the molar ratio of hydrogen to oxygen is between about 1.3 to 2.7 but preferably above 1.9. The pressure of the hydrogen-oxygen mixture is above atmospheric before initiating the reticulation porcess. In another embodiment, combustion reticulation is followed by an annealing period. In any of these combustion reticulation embodiments, the reticulated foam can optionally be washed. In any of these combustion reticulation embodiments, the reticulated foam can optionally be dried.

In one embodiment, the reticulated elastomeric matrix's permeability to a fluid, e.g., a liquid, is greater than the permeability to the fluid of an unreticulated matrix from which the reticulated elastomeric matrix was made. In another embodiment, the reticulation process is conducted to provide an elastomeric matrix configuration favoring cellular ingrowth and proliferation into the interior of the matrix. In another embodiment, the reticulation process is conducted to provide an elastomeric matrix configuration which favors cellular ingrowth and proliferation throughout the elastomeric matrix configured for implantation, as described herein.

In certain exemplary embodiments, reticulated elastomeric matrices comprising polycarbonate polyurethane or polycarbonate polyurethane urea are contemplated to be particularly suitable. Specifically, the reticulated elastomeric matrix may be made from a single sheet of reticulated polycarbonate polyurethane. The polycarbonate polyurethane may comprise an isocyanate component and a polycarbonate polyol component. Exemplary isocyanate components may contain 2,4′diphenylmethane diisocyanate (“2,4′-MDI”), 4,4′diphenylmethane diisocyanate (4,4′-MDI), or a mixture thereof. Preferably, the isocyanate component contains a mixture of at least about 5%, and more preferably about 5% to about 50%, by weight of 2,4′-MDI with the balance 4,4′-MDI. The isocyanate index is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98.

In certain embodiments, the matrix has a void content greater than 90% with average cell sizes measuring in the range of 250 to 500 microns.

The elastomeric matrix that incorporates the fibers into the reticulated cross-linked biodurable elastomeric polycarbonate urea-urethane matrix can vary in its density and/or in its orientation. The density of the elastomeric matrix can vary, in one embodiment from about 2 lbs/ft³ to about 25 lbs/ft³ (from about 0.032 g/cc to about 0.401 g/cc), from about 2.5 lbs/ft³ to about 10 lbs/ft³ (from about 0.040 g/cc to about 0.160 g/cc) in another embodiment, or from about 3 lbs/ft³ to about 8.5 lbs/ft³ (from about 0.480 g/cc to about 0.136 g/cc) in another embodiment. Orientation can occur during initial formation of foam, during reticulation, or during secondary processing that may occur after reticulation and thermal curing of the foam. The results of orientation are manifested by enhanced properties and/or enhanced performance in the direction of orientation. In one embodiment, a device made from a reinforced reticulated elastomeric matrix is positioned in the tissue being repaired in such a way that the enhanced properties and/or enhanced performance of the oriented matrix is aligned in the direction to resist the higher load bearing direction. Incorporation of the reinforcement may lead to enhanced performance of the matrix, which is superior to that which would be obtained by orienting the reinforced matrix in one or more directions.

Certain embodiments of the invention comprise a biostable cross-linked reticulated resilient elastomeric matrix made from polycarbonate polyurethane-urea with a morphology composed of continuous interconnected and intercommunicating pores. The matrix is made, for example, by a polymerization reaction between aromatic isocyanate and polycarbonate polyol in the presence of chain extenders, cross-linking agent, surfactants, catalysts and processing aids. This reaction leads to the formation of a segmented polyurethane polymer with hard and soft segments. The polymerization reaction is accompanied by a second reaction between aromatic isocyanate and water, which produces the urea bonds or segments with simultaneous formation of carbon dioxide (CO₂). Release of the CO₂ aids in the formation of a porous material with cellular structure. The membranes between the cellular walls formed during the polymerization reaction are removed to provide an inter-communicating and inter-connected porous structure. Both chemical and physical cross-links are present in this material. The segmented and cross-linked material formed is elastomeric and demonstrates resilient recovery after being deformed under both compression and tension.

### Imparting Endopore Features

Within pores 20, elastomeric matrix 10 or composite mesh comprising reticulated elastomeric matrix 10 may, optionally, have features in addition to the void or gas-filled volume described above. In one embodiment, elastomeric matrix 10 or composite mesh comprising reticulated elastomeric matrix 10 may have what are referred to herein as “endopore” features as part of its microstructure, i.e., features of elastomeric matrix 10 that are located “within the pores”. In one embodiment, the internal surfaces of pores 20 may be “endoporously coated”, i.e., coated or treated to impart to those surfaces a degree of a desired characteristic, e.g., hydrophilicity. The coating or treating medium can have additional capacity to transport or bond to active ingredients that can then be preferentially delivered to pores 20. In one embodiment, this coating medium or treatment can be used facilitate covalent bonding of materials to the interior pore surfaces, for example, as are described in the applications to which priority is claimed. In another embodiment, the coating comprises a biodegradable or absorbable polymer and an inorganic component, such as hydroxyapatite. Hydrophilic treatments may be effected by chemical or radiation treatments on the fabricated reticulated elastomeric matrix 10 or composite mesh comprising reticulated elastomeric matrix 10, by exposing the elastomer to a hydrophilic, e.g., aqueous, environment during elastomer setting, or by other means known to those skilled in the art.

Furthermore, one or more coatings may be applied endoporously by contacting with a film-forming biocompatible polymer either in a liquid coating solution or in a melt state under conditions suitable to allow the formation of a biocompatible polymer film on reticulated elastomeric matrix 10 or composite mesh comprising reticulated elastomeric matrix 10. In one embodiment, biocompatible polymer films can be first made from a melt state or casting from a solution state before incorporating them with the biodurable reticulated elastomeric matrix using various processing techniques known in the art including adhesive bonding, melt processing, compression molding, solution casting, thermal bonding, suturing, and other techniques. In one embodiment, the polymers used for such coatings are film-forming biocompatible polymers with sufficiently high molecular weight so as not to be waxy or tacky. The polymers should also adhere to the solid phase 12. In another embodiment, the bonding strength is such that the polymer film does not crack or dislodge during handling or deployment of reticulated elastomeric matrix 10 or composite mesh comprising reticulated elastomeric matrix 10. In one embodiemnt, one or more coatings that may be applied endoporously may have anti-adhesion properties. The coating or coatings can act as or impart anti-adhesion functionality in repair of some soft tissue defects such as in a number of hernia applications. The coating is important to impart anti-adhesion functionality, and is especially important in anatomic sites such as abdominal wall wherein adhesions are likely to form between internal organ structures and the exposed mesh surface.

In one embodiment, one or more coatings that may be applied endoporously and may have anti-adhesion properties need not necesasarily form a polymer film or a continuous polymer film. In another embodiment, one or more coatings that may be applied endoporously and may have anti-adhesion properties may coat the the internal surfaces of pores 20. In one embodiment, the internal surfaces of pores 20 may be “endoporously coated”, i.e., coated or treated to impart to those surfaces a degree of a desired characteristic, e.g., have anti-adhesion properties or have anti-adhesion barrier.

Suitable biocompatible polymers include polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone or a mixture thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules or a mixture thereof. For the purpose of this invention aliphatic polyesters include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one or a mixture thereof. In one embodiment, the reinforcement can be made from biopolymer, such as collagen, elastin, and the like. The biopolymer can be biodegradable or bioabsorbable. Biodegradable or bioabsorbable coatings made from copolymers of caprolactone with lactic acid, glycolic acid, acid d-, l- and meso lactide and para-dioxanone are considered favorable for coating applications for providing anti-adhesion properties with copolymers of caprolactone with lactic acid in the the ratio of 40/60, 30/70 or 20/80 polycaprolactone to polylactic acid being prfrred for anti-adhesion properties. In another embodiment, biodegradable or bioabsorbable coatings comprise copolymers of caprolactone, lactic acid, glycolic acid, acid d-, l- and meso lactide and para-dioxanone, etc. or mixtures thereof para-dioxanone Further, the thermoplastic biodegradable or bioabsorbable polymer used for coating may comprise an ε-caprolactone copolymer, and optionally an ε-caprolactone-lactic acid copolymer or an ε-caprolactone-lactide copolymer.

Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from cross-linked polyvinyl pyrrolidinone and polyesters. Other polymers can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydoxyalkyl celluloses); or a mixture thereof. For the purpose of this invention, polyamides include polyamides of the general forms:

—N(H)—(CH₂)ₙ—C(O)— and —N(H)—(CH₂)ₓ—N(H)—C(O)—(CH₂)_{y}—C(O)—,

where n is an integer from about 4 to about 13; x is an integer from about 4 to about 12; and y is an integer from about 4 to about 16. It is to be understood that the listings of materials above are illustrative but not limiting.

In another embodiment, biocompatible polymers further include film-forming biodurable polymers with relatively low chronic tissue response, such as polycarbonate polyurethanes, polysiloxane polyurethanes, poly(siloxane-co-ether) polyurethanes, polycarbonate polysiloxane polyurethanes, polycarbonate urea-urethanes, polycarbonate polysiloxane urea-urethanes and the like and their mixtures.

A device such as a composite mesh made from reticulated elastomeric matrix 10 generally is coated by simple dip or spray coating with a polymer, optionally comprising a pharmaceutically-active agent, such as a therapeutic agent or drug. In one embodiment, the coating is a solution and the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight. In another embodiment, the coating may be applied as a solution in a solvent for the polymer, for example, with a polymer content in the coating solution of from about 1% to about 40% by weight. According to other embodiments, the coating solution may be applied by dip coating or spray coating the solution onto the reticulated elastomeric matrix, the solvent can be substantially or completely removed from the coating, and/or the solvent may be non-toxic and non-carcinogenic.

The solvent or solvent blend for the coating solution is chosen with consideration given to, inter alia, the proper balancing of viscosity, deposition level of the polymer, wetting rate and evaporation rate of the solvent to properly coat solid phase 12, as known to those in the art. In one embodiment, the solvent is chosen such the polymer is soluble in the solvent. In another embodiment, the solvent is substantially completely removed from the coating. In another embodiment, the solvent is non-toxic, non-carcinogenic and environmentally benign. Mixed solvent systems can be advantageous for controlling the viscosity and evaporation rates. In all cases, the solvent should not react with the coating polymer. Solvents include by are not limited to: acetone, N-methylpyrrolidone (“NMP”), DMSO, toluene, methylene chloride, chloroform, 1,1,2-trichloroethane (“TCE”), various freons, dioxane, ethyl acetate, hexane, heptane, other liquid hydrocarbon THF, DMF and DMAC.

In another embodiment, the film-forming coating polymer is a thermoplastic polymer that is melted, enters the pores 20 of the elastomeric matrix 10 or composite mesh comprising reticulated elastomeric matrix 10 and, upon cooling or solidifying, forms a coating on at least a portion of the solid material 12 of the elastomeric matrix 10. In other embodiments, a thermoplastic polymer is melted and applied to coat the reticulated elastomeric matrix. In another embodiment, the processing temperature of the thermoplastic coating polymer in its melted form is above about 60° C. In another embodiment, the processing temperature of the thermoplastic coating polymer in its melted form is above about 90° C. In another embodiment, the processing temperature of the thermoplastic coating polymer in its melted form is above about 120° C. The melt can be applied by extruding or coextruding or injection molding or compression molding or compressive molding the melt onto the reticulated elastomeric matrix. In other embodiments, the coating is formed into a film, and is then bonded to the implantable device using an adhesive, such as Nusil™, Chronoflex™, Elast-Eon™ or a biodegradable polymer.

In a further embodiment of the invention, described in more detail below, some or all of the pores 20 of elastomeric matrix 10 or composite mesh comprising reticulated elastomeric matrix 10 are coated or filled with a cellular ingrowth promoter. In another embodiment, the promoter can be foamed. In another embodiment, the promoter can be present as a film. The promoter can be a biodegradable or absorbable material to promote cellular invasion of elastomeric matrix 10 in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of elastomeric matrix 10 is coated or impregnated, as described in the previous section but substituting the promoter for the biocompatible polymer or adding the promoter to the biocompatible polymer, to encourage cellular ingrowth and proliferation.

### Reinforcements

A second component of the implantable device of the present invention is a support structure for reinforcing the mechanical properties of the implantable device. In one embodiment of the invention, the implantable device comprises a reticulated elastomeric matrix that is reinforced with a reinforcement to create a composite structure, such as a composite mesh. The reinforced reticulated elastomeric matrix and/or composite mesh may be made more functional for specific uses in various implantable devices by including or incorporating a support structure, such as a reinforcement (e.g., fibers) with or into the matrix, preferably, a reticulated cross-linked biodurable elastomeric polycarbonate urea-urethane matrix.

Incorporation of the support structure, such as a reinforcement (e.g., fibers, fiber meshes, wires and/or sutures) or more than one reinforcement with or into an reticulated elastomeric matrix may impart enhanced functionalities. The reinforcement may be designed to enhance the mechanical load bearing functions of said implantable device, which include strength, stiffness, tear resistance, burst strength, suture pullout strength, etc. For example, the enhanced functionalities that can be imparted by using a reinforcement include but are not limited to enhancing the ability of the device to withstand pull out loads associated with suturing during surgical procedures, the device's ability to be positioned at the repair site by suture anchors during a surgical procedure, and holding the device at the repair site after the surgery when the tissue healing takes place. In another embodiment, the enhanced functionalities provide additional load bearing capacities to the device during surgery in order to facilitate the repair or regeneration of tissues. In another embodiment, the enhanced functionalities provide additional load bearing capacities to the device, at least through the initial days following surgery, in order to facilitate the repair or regeneration of tissues. In another embodiment, the enhanced functionalities provide additional load bearing capacities to the device following surgery in order to facilitate the repair or regeneration of tissues. In one embodiment, the reinforcement used does not interfere with the matrix's capacity to accommodate tissue ingrowth and proliferation.

In a particular embodiment, the reinforcement is in at least one dimension, e.g., a 1-dimensional reinforcement (such as a fiber), a 2-dimensional reinforcement (such as a 2-dimensional mesh made up of intersecting 1-dimensional reinforcement elements), or a 3-dimensional reinforcement (such as a 3-dimensional grid). In other embodiments, the reinforcement comprises a medical grade textile.

Embodiments of the invention provide, for example, an implantable device comprising a reticulated resiliently-compressible elastomeric matrix having a plurality of pores, wherein the implantable device further comprises a reinforcement in at least one dimension. In embodiments of the invention, the reinforcement may comprise a two-dimensional reinforcement, and the two-dimensional reinforcement may further comprise a grid of a plurality of one-dimensional reinforcement elements, wherein the one-dimensional reinforcement elements cross each other's paths. In further embodiments, the two-dimensional reinforcement may be a two-dimensional mesh having intersecting one-dimensional reinforcement elements.

The reinforcement can comprise mono-filament fiber, multi-filament yarn, braided multi-filament yarns, commingled mono-filament fibers, commingled multi-filament yarns, bundled mono-filament fibers, bundled multi-filament yarns, and the like. The reinforcement can comprise an amorphous polymer, semi-crystalline polymer, e.g., polyester or nylon, carbon, e.g., carbon fiber, glass, e.g., glass fiber, ceramic, cross-linked polymer fiber and the like or any mixture thereof. The fibers can be made from absorbable or non-absorbable materials. In one embodiment, the fiber reinforcement of the present invention is made from a biocompatible material(s).

In one embodiment, the reinforcement can comprise at least one non-absorbable material, such as a non-biodegradable or non-absorbable polymer. Examples of suitable non-absorbable polymers include but are not limited to polyesters (such as polyethylene terephthalate and polybutylene terephthalate); polyolefins (such as polyethylene and polypropylene including atactic, isotactic, syndiotactic, and blends thereof as well as, polyisobutylene and ethylene-alpha-olefin copolymers); acrylic polymers and copolymers; vinyl halide polymers and copolymers (such as polyvinyl chloride); polyvinyl ethers (such as polyvinyl methyl ether); polyvinylidene halides (such as polyvinylidene fluoride and polyvinylidene chloride); polyacrylonitrile; polyvinyl ketones; polyvinyl aromatics (such as polystyrene); polyvinyl esters (such as polyvinyl acetate); copolymers of vinyl monomers with each other and olefins (such as etheylene-methyl methacrylate copolymers, acrylonitrile-styrene copolymers, ABS resins and ethylene-vinyl acetate copolymers); polyamides (such as nylon 4, nylon 6, nylon 66, nylon 610, nylon 11, nylon 12 and polycaprolactam); alkyd resins; polycarbonates; polyoxymethylenes; polyimides; polyethers; epoxy resins; polyurethanes; rayon; rayon-triacetate; and any mixture thereof. Polyamides, for the purpose of this application, also include polyamides of the form —NH—(CH₂)ₙ—C(O)— and —NH—(CH₂)ₓ—NH—C(O)—(CH₂)_{y}—C(O)—, wherein n is an integer from 6 to 13 inclusive; x is an integer from 6 to 12 inclusive; and y is an integer from 4 to 16 inclusive.

In another embodiment, the reinforcement can comprise at least one biodegradable, bioabsorbable or absorbable polymer. Examples of suitable absorbable polymers include but are not limited to aliphatic polyesters, e.g., homopolymers and copolymers of lactic acid, glycolic acid, lactide, glycolide, para-dioxanone, trimethylene carbonate, ε-caprolactone and blends thereof. Further exemplary biocompatible polymers include film-forming bioabsorbable polymers such as aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules, and any mixture thereof. Aliphatic polyesters, for the purpose of this application, include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-gone, 1,5-dioxepan-2-gone, 6,6-dimethyl-1,4-dioxan-2-gone, and any mixture thereof.

Such fiber(s)/yarn(s) can be made by melt extrusion, melt extrusion followed by annealing and stretching, solution spinning, electrostatic spinning, and other methods known to those in the art. Each fiber can be bi-layered, with an inner core and an outer sheath, or multi-layered, with inner core, an outer sheath and one or more intermediate layers. In bi- and multi-layered fibers, the core, the sheath or any layer(s) outside the core can comprise a degradable or dissolvable polymer. The fibers can be uncoated or coated with a coating that can comprise an amorphous polymer, semi-crystalline polymer, carbon, glass, ceramic, and the like or any mixture thereof.

Alternatively, the reinforcement can be made from carbon, glass, a ceramic, bioabsorbable glass, silicate-containing calcium-phosphate glass, or any mixture thereof. The calcium-phosphate glass, the degradation and/or absorption time in the human body of which can be controlled, can contain metals, such as iron, magnesium, sodium, potassium, or any mixture thereof.

In another embodiment, the one-dimensional reinforcement comprises an amorphous polymer fiber, a semi-crystalline polymer fiber, a cross-linked polymer fiber, a biopolymer fiber, a collagen fiber, an elastin fiber, carbon fiber, glass fiber, bioabsorbable glass fiber, silicate-containing calcium-phosphate glass fiber, ceramic fiber, polyester fiber, nylon fiber, an amorphous polymer yarn, a semi-crystalline polymer yarn, a cross-linked polymer yarn, a biopolymer yarn, a collagen yarn, an elastin yarn, carbon yarn, glass yarn, bioabsorbable glass yarn, silicate-containing calcium-phosphate glass yarn, ceramic yarn, polyester yarn, nylon yarn, or any mixture thereof.

In certain embodiments, the one-dimensional reinforcement of the two-dimensional mesh may comprise mono-filament fiber, multi-filament yarn, braided multi-filament yarns, commingled mono-filament fibers, commingled multi-filament yarns, bundled mono-filament fibers, bundled multi-filament yarns, or any mixture thereof. In another embodiment, the two-dimensional reinforcement comprises intersecting one-dimensional reinforcement elements comprising an amorphous polymer fiber, a semi-crystalline polymer fiber, a cross-linked polymer fiber, a biopolymer fiber, carbon fiber, glass fiber, bioabsorbable glass fiber, silicate-containing calcium-phosphate glass fiber, ceramic fiber, polyester fiber, nylon fiber, an amorphous polymer yarn, a semi-crystalline polymer yarn, a cross-linked polymer yarn, a biopolymer yarn, carbon yarn, glass yarn, bioabsorbable glass yarn, silicate-containing calcium-phosphate glass yarn, ceramic yarn, polyester yarn, nylon yarn, or any mixture thereof. According to certain embodiments of the invention, the one-dimensional reinforcement of the two-dimensional mesh comprises one or more absorbable materials, such as any one or more of a homopolymer or copolymer of lactic acid, lactide, and ε-caprolactone, for example, a lactic acid homopolymer, an ε-caprolactone-lactic acid copolymer or an ε-caprolactone-lactide copolymer. In other embodiments, the one-dimensional reinforcement of the two-dimensional mesh comprises at least one non-absorbable material, such as a polyolefin, for example, polypropylene.

In further embodiments, the one-dimensional reinforcement of the two-dimensional mesh is uncoated. In still further embodiments, the one-dimensional reinforcement of the two-dimensional mesh is coated with a polymer. In one exemplary embodiment, the one-dimensional reinforcement of the two-dimensional mesh can be uncoated or coated with a polymer, and/or the absorbable material can be a lactic acid homopolymer coated with a coating comprising an ε-caprolactone copolymer, such as an ε-caprolactone-lactic acid copolymer or an ε-caprolactone-lactide copolymer.

The reinforcement can be incorporated into the reticulated elastomeric matrix in different patterns. In one embodiment, the reinforcement is placed along an entire surface or a contact surface of the elastomeic matrix, said surface or contact surface may be one of two opposing sides to said reticulated elastomeric matrix. In one embodiment, the reinforcement is placed along the border of the device, maintaining a fixed distance from the device's edges. In another embodiment, the reinforcement is placed along the border of the device, maintaining a variable distance from the device's edges. In another embodiment, the reinforcement is placed along the perimeter, e.g., circumference for a circular device, of the device, maintaining a fixed distance from the device's edges. In another embodiment, the reinforcement is placed along the perimeter of the device, maintaining a variable distance from the device's edges. In another embodiment, the reinforcement is present as a plurality of parallel and/or substantially parallel 1-dimensional reinforcement elements, e.g., as a plurality of parallel lines such as parallel fibers. In another embodiment, the reinforcement is placed as a 2- or 3-dimensional reinforcement grid in which the 1-dimensional reinforcement elements cross each other's path. In another embodiment, the reinforcement is placed as a 2- or 3-dimensional reinforcement grid in which the 1-dimensional reinforcement elements cross each other's path, but reinforcement is not placed along the perimeter or border of the device. The grid can have one or multiple reinforcement elements. In 2- or 3-dimensional reinforcement grid embodiments, the elements of the reinforcement can be arranged in geometrically-shaped patterns, such as square, rectangular, trapezoidal, triangular, diamond, parallelogram, circular, eliptical, pentagonal, hexagonal, and/or polygons with seven or more sides. The reinforcement elements comprising a reinforcement grid can all be of the same shape and size or can be of different shapes and sizes. The reinforcement elements comprising a reinforcement grid can additionally include border, perimeter and/or parallel line elements. The performance or properties of the reinforcement grid incorporates the reinforcement into the matrix and the thus-reinforced matrix depends on the inherent properties of the reinforcement as well as the pattern, geometry and number of elements of the grid.

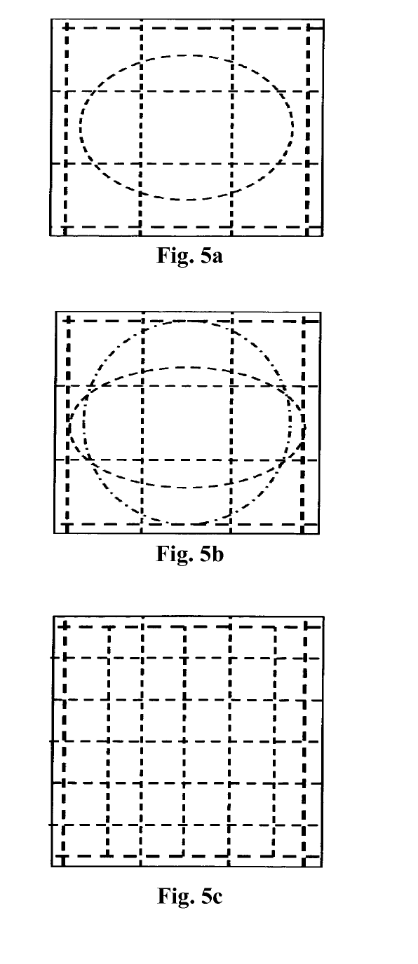

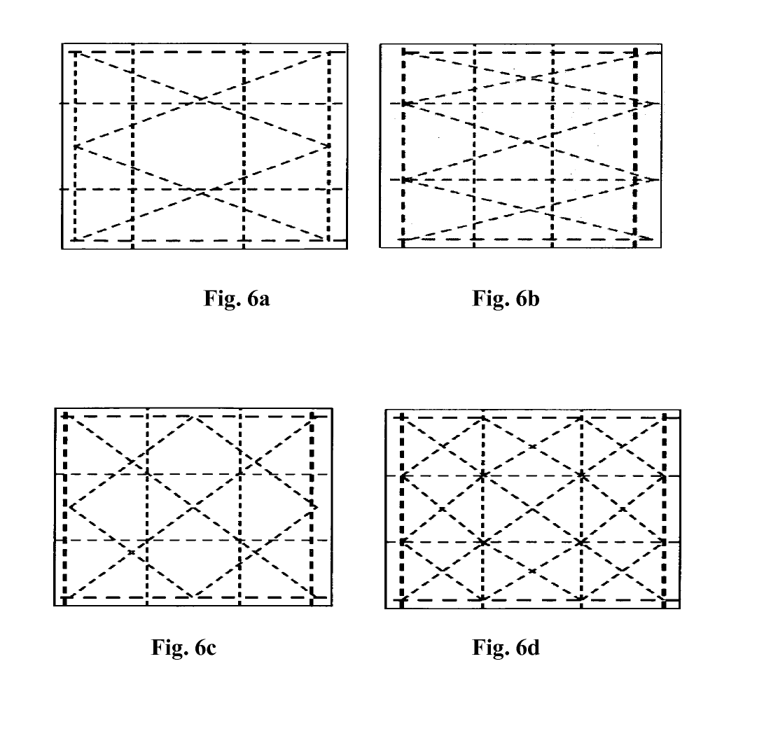

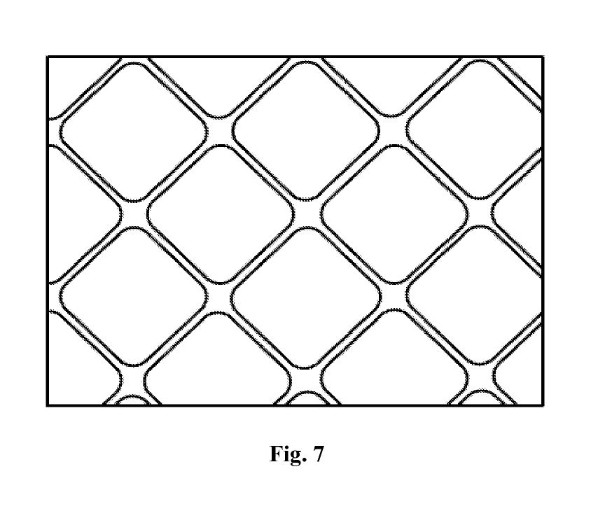

Some exemplary, but not limiting, reinforcement grids are illustrated in FIGS. 5 and 6. Each of FIGS. 5a-5c and 6a-6d include include a border or perimeter reinforcing element or elements. FIG. 5a illustrates an eliptical reinforcement element superimposed on a rectangular grid reinforcement element. FIG. 5b illustrates two eliptical reinforcement elements superimposed on a rectangular grid reinforcement element. FIG. 5c illustrates a rectangular grid reinforcement element. FIG. 6a illustrates a diamond-shaped grid reinforcement element superimposed on a rectangular grid reinforcement element. FIG. 6b illustrates a 4-sided polygional-shaped grid reinforcement element superimposed on a rectangular grid reinforcement element. FIGS. 6c and 6d illustrate diamond-shaped grid reinforcement elements of different spacing and diagional reinforcement elements superimposed on a rectangular grid reinforcement element. FIG. 7 illustrate a grid or a two dimensional reinforcement.

In one embodiment, any one of the edges of a single grid element can be from about 0.25 mm to about 20 mm long, or from about 5 mm to about 15 mm long in another embodiment.

In other embodiments, the clearance or spacing between reinforcement elements, such as the clearance between adjacent linear reinforcement elements, can be from about 0.25 mm to about 20 mm in one embodiment, or from about 0.5 mm to about 15 mm in another embodiment. In other embodiments, the clearance between reinforcement elements is substantially the same between elements. In other embodiments, the clearance between reinforcement elements differs between different elements. In other multi-dimensional reinforcement embodiments, the clearance between reinforcement elements in one dimension is independent of the clearance(s) between reinforcement elements in any other dimension.

The diameter of a reinforcement element having a substantially circular cross-section can be from about 0.03 mm to about 0.50 mm in one embodiment, or from about 0.07 mm to about 0.30 mm in another embodiment, or from about 0.05 mm to about 1.0 mm in another embodiment, or from about 0.03 mm to about 1.0 mm in another embodiment. In another embodiment, the diameter of a reinforcement element having a substantially circular cross-section can be equivalent to a USP suture diameter from about size 8-0 to about size 0 in one embodiment, from about size 8-0 to about size 2 in another embodiment, from about size 8-0 to about size 2-0 in another embodiment.

In specific embodiments of the present invention, one-dimensional reinforcement of the two-dimensional mesh can have a substantially circular cross-section with a diameter of from about 0.03 mm to about 1.0 mm, and optionally from about 0.07 mm to about 0.30 mm. According to embodiments of the invention, the edges of the grid elements of the two-dimensional mesh formed from one-dimensional reinforcement elements may be from about 0.25 mm to about 20 mm long, and optionally from about 5 mm to about 15 mm long.

The reinforcement layout or the distribution and pattern of reinforcement elements, e.g., fibers or sutures or grid, in the matrix will depend on design requirement and/or the application for which the device will be used.

### Composite Device

A device according to embodiments of the invention can be made from a reticulated elastomeric matrix comprising a plurality of pores (the pores may be interconnected and intercommunicating open pores, forming a network that permits tissue in-growth and proliferation into the implant), or from separate pieces of reticulated elastomeric matrices with the addition of a medical grade textile and optional anti-adhesion coating(s) or barriers. In certain embodiments the reticulated elastomerix matrix may be compressed. A particularly preferred embodiment of the implantable device of the present invention comprises a non-absorbable mesh manufactured from a polycarbonate polyurethane-urea matrix and knitted polypropylene monofilament fibers.

In certain embodiments of the implantable device, the device is a composite of a reticulated elastomeric matrix and a mesh material. Embodiments of the invention provide composite mesh devices intended for repair of soft tissue defects, comprising a reticulated elastomeric matrix which is designed to support tissue ingrowth, and at least one functional element. Such functional elements may impart functionalities to the composite device including mechanical reinforcement and strength, anti-adhesion, device orientation, shape memory, and enhanced healing. One such functional element is a medical grade textile used as a reinforcement to impart biaxial mechanical strength. Such textiles may either be permanent (e.g., polyester mesh, polypropylene mesh) or resorbable (e.g., polylactic acid, poly (lactide ε-capralactone).

Another such functional element is a thin layer, coating or film of either a permanent polymer or biodegradable polymer used to reduce the potential for biological adhesions. Other functional elements which may be incorporated with the reticulated elastomeric matrix to form a composite device include biologically derived collagen meshes (xenografts, allografts) used to enhance tissue response and minimize adhesion; polymeric and/or metallic structures used to impart shape memory; and markers including dyes used to differentiate between two sides of a mesh which may have differing characteristics. Any of these preferred functional elements may be incorporated with the biodurable reticulated elastomeric matrix using various processing techniques known in the art including adhesive bonding, melt processing, compression molding, suturing, and other techniques.

Composite mesh embodiments include several different geometries for different anatomic applications. One particular embodiment of the invention includes a “sandwich design” wherein a medical grade textile can be incorporated between two layers of a biodurable reticulated matrix. Another embodiment of the invention includes an “open face sandwich design” wherein a medical grade textile is incorporated with a single layer of the biodurable reticulated matrix. With either construct, an optional anti-adhesion coating can be added to one or both surfaces, particularly opposing faces of the composite device. In another embodiment, multiple layers of reinforcement and elastomeric matrix can be stacked in an alternating fashion and an adhesive can be used to incorporate the alternating layer. The resulting composite mesh can be further functionalized to render bioactive properties such as antimicrobial action, release of cytokines, growth factors, and other promoters of cellular activity, angiogenesis, and extracellular matrix synthesis.

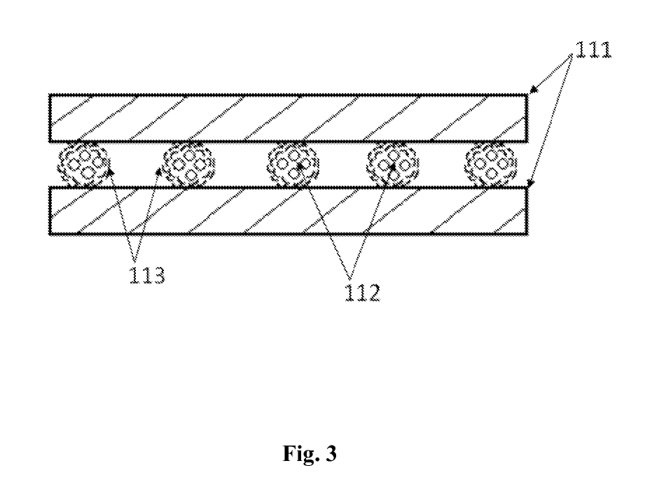

FIG. 3 shows a schematic of the “sandwich design” or a composite where the 2-dimensional mesh reinforcement (112) is attached to two layers of elastomeric matrix (111) using an adhesive (113). One embodiment of the composite surgical mesh is a “sandwich design” fabricated using two layers of a biostable, reticulated (possessing interconnected and intercommunicating open pores), elastomeric resilient matrix made from a polycarbonate polyurethane, and a support structure, which may be a lightweight polypropylene fiber mesh. The matrix has a unique reticulated architecture, defined as an open-celled, porous structure with an interconnected and intercommunicating network of pores that permits tissue in-growth and proliferation into the implant. The support structure may include any structure reinforcing the mechanical properties of the device, which includes filaments, fibers, other supporting struts or frames in any shape or arrangement, such as, for example, a one dimensional arrangement, a two dimensional (e.g., cross-over arrangement), or an interwoven mesh. Other exemplary support structures and arrangement may be any of the structures and arrangement disclosed in U.S. Patent Application Publication No. 2007/0190108, the disclosures of which are hereby incorporated by reference.

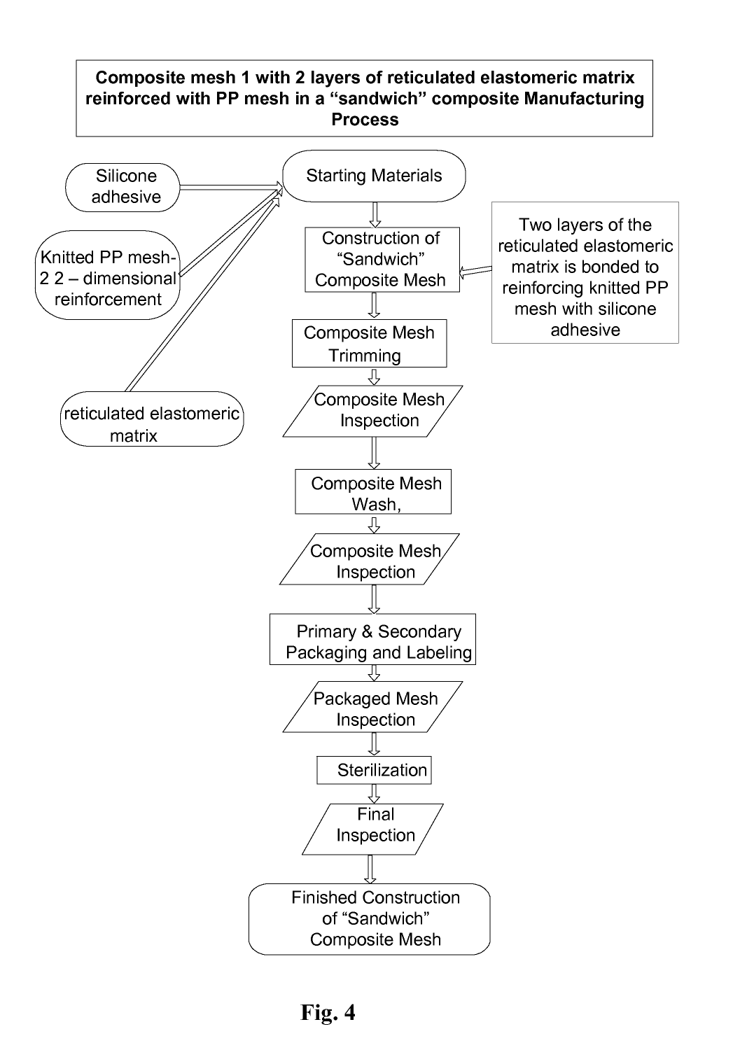

FIG. 4 shows a schematic of manufacturing a “sandwich design” or a composite where the 2-dimensional mesh reinforcement is attached to two layers of elastomeric matrix using an adhesive starting from initial raw materials to the finished product.

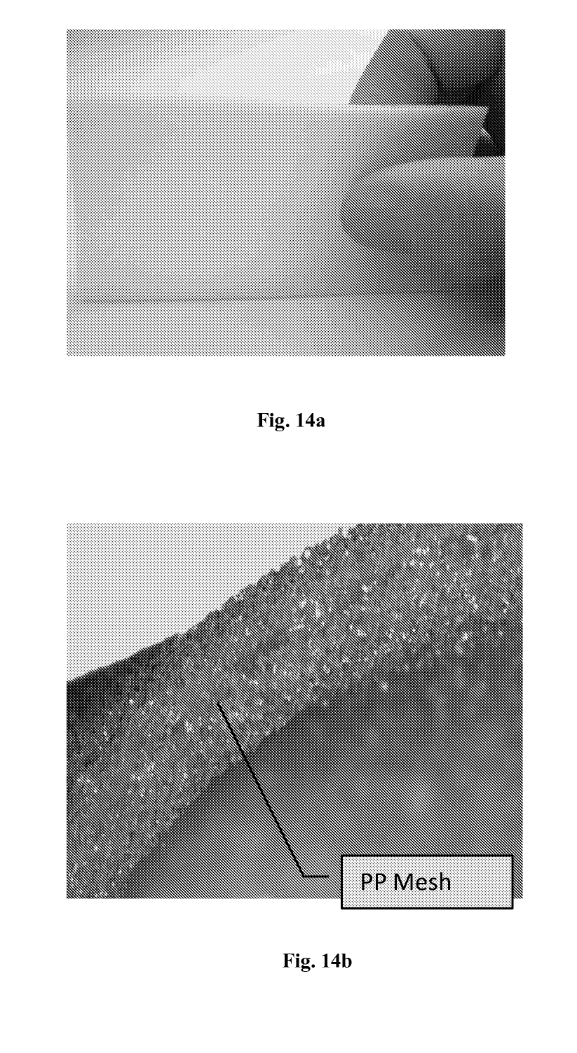

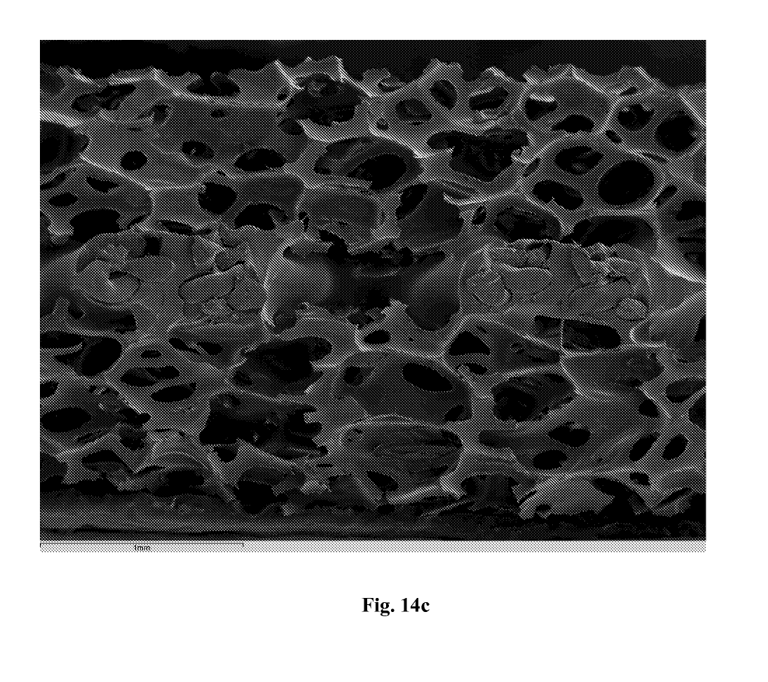

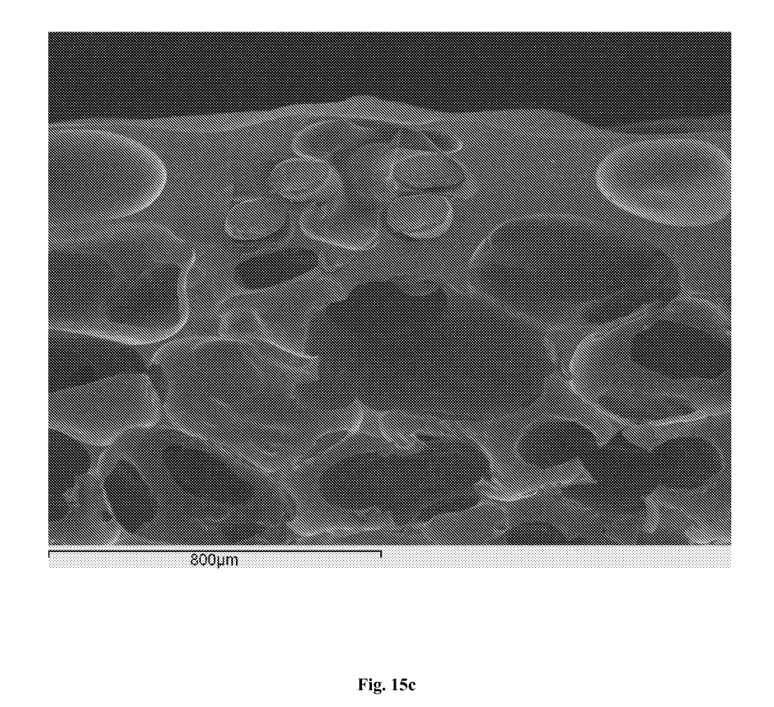

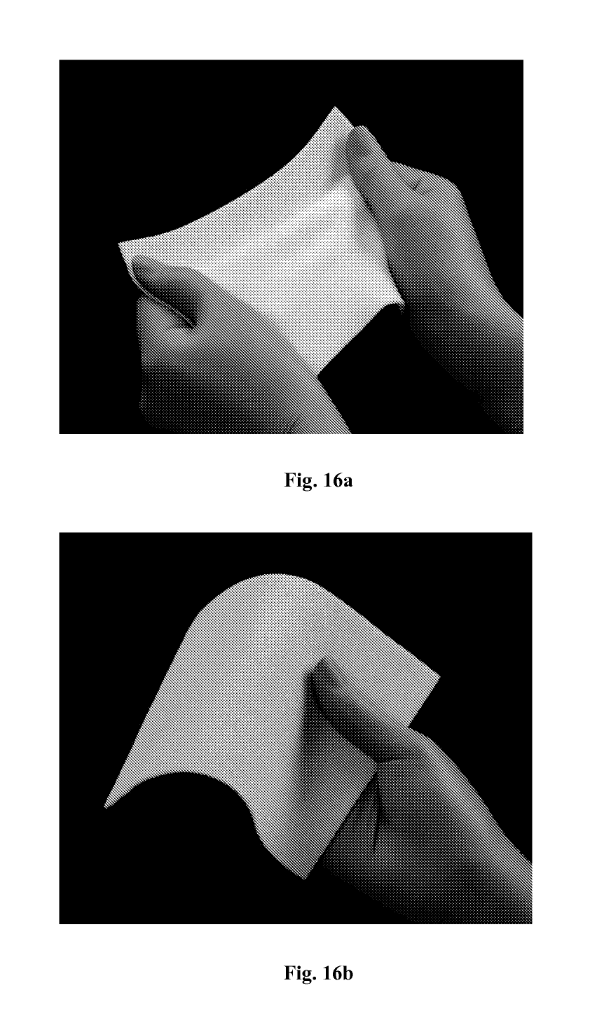

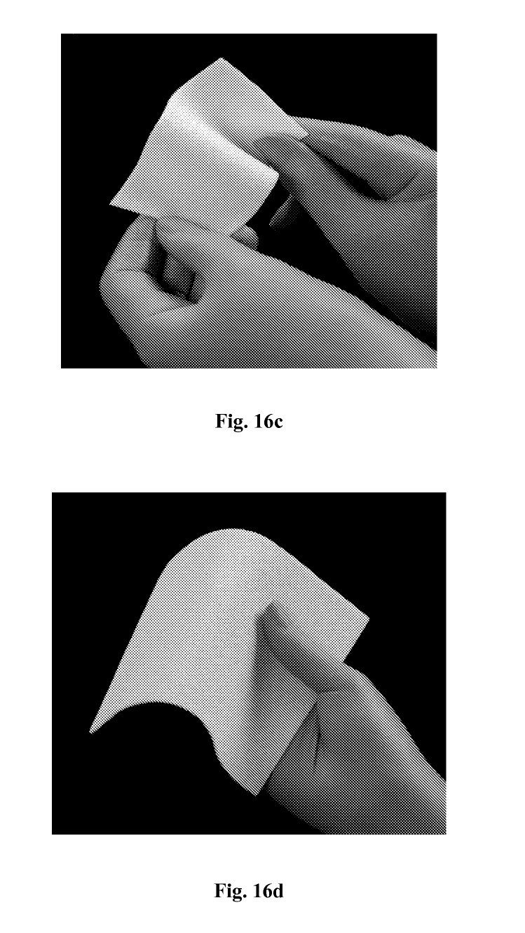

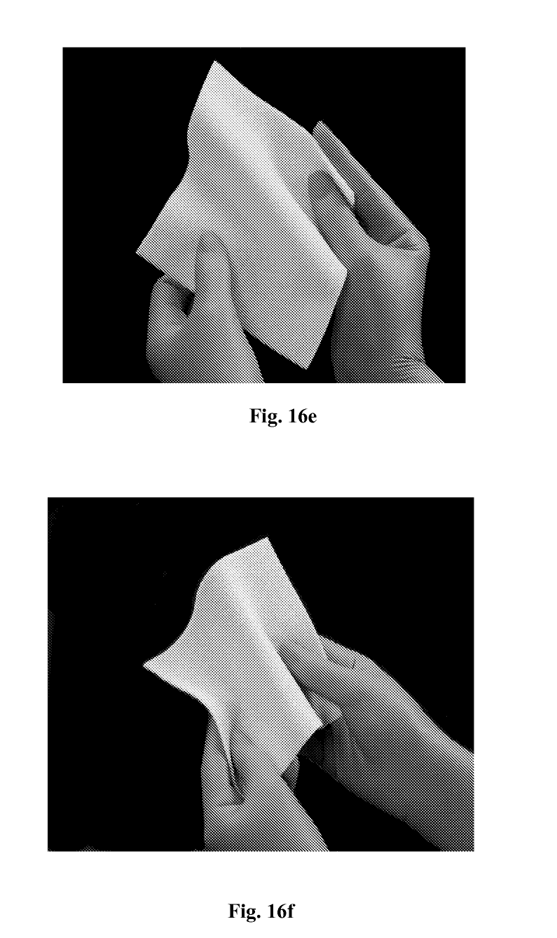

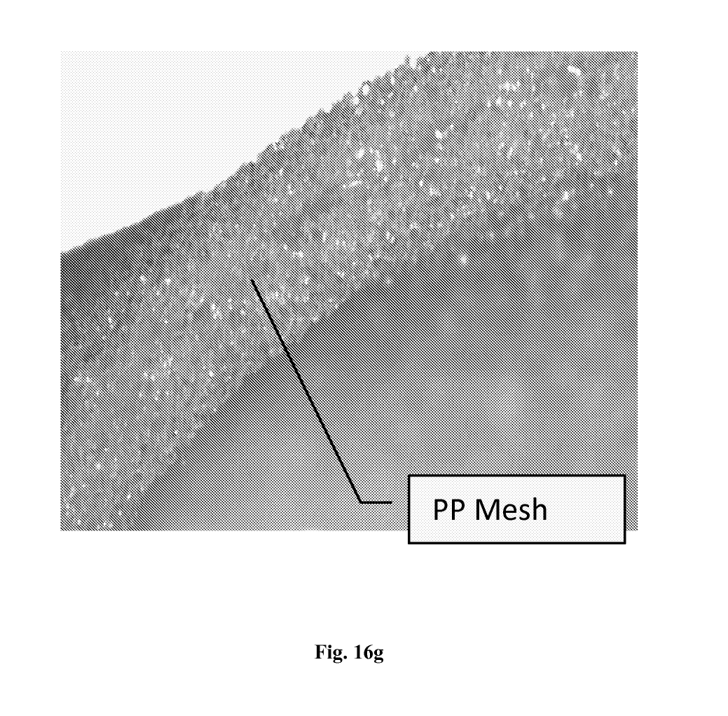

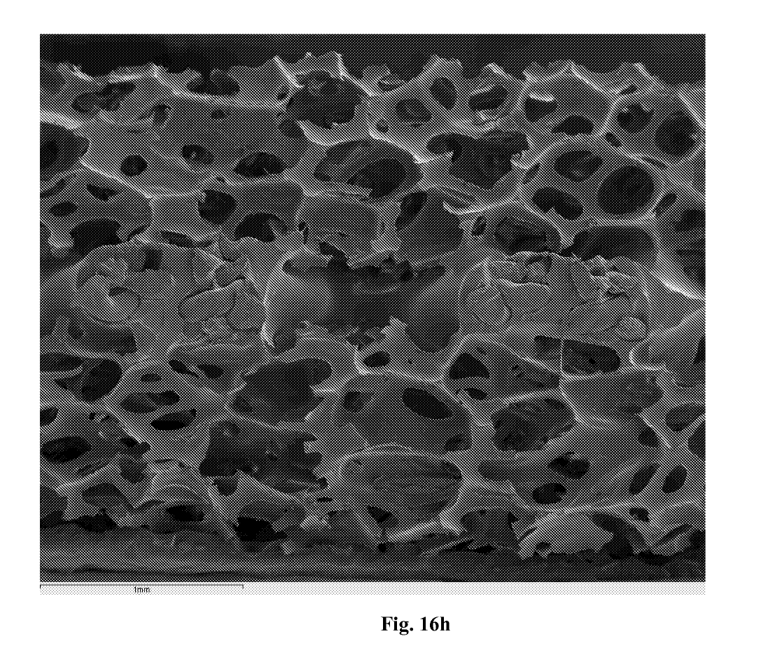

In one exemplary embodiment, a polypropylene (PP) mesh (knitted polypropylene monofilament fibers) is sandwiched between the two layers of a polycarbonate polyurethane reticulated matrix. Preferably, the matrix is fully reticulated with a void content of greater than 90% and cell sizes in the range of 250 to 500 microns. Silicone adhesive (Nusil™ MED2-4213) is used to bond the polypropylene mesh to the two sheets of polycarbonate polyurethane substrates. FIGS. 14a-14c, and 16a-16h illustrate examples of such sandwich design. Mechanical testing has shown this design is substantially equivalent to predicate devices (Mersilene™) while providing the biological advantages of the three dimensional construct to facilitate faster healing.

Various other medical grade textiles may be used to form composite devices according to embodiments of the invention, including for example textiles made from biodurable polymers such as polypropylene, polyester, PTFE, or mixtures of these polymers. The medical grade textiles can be made from biodegradable polymers such as PLA, PGA, Caprolactone, and said copolymers of biodegradable polymer such as PLA-PGA, PLA-Caprolactone, etc.

In one embodiment, in some applications, such as rotator cuff repair or repair of soft tissue defects such as number of hernia applications where the implantable device serves in an augmentary role, precise fitting may not be required to match or fit the tissue that is being repaired or regenerated. In another embodiment, an implantable device containing a reinforced reticulated elastomeric matrix is shaped prior to its use, such as in surgical repair of tendons and ligaments or in repair of soft tissue defects, specifically inguinal, femoral, ventral, incisional, umbilical, and epigastric hernias; meshes for tissue augmentation, support and repair. One exemplary method of shaping is trimming. When shaping is desired, the reinforced reticulated elastomeric matrix can be trimmed in its length and/or width direction along the lines or reinforcing fibers. In one embodiment, this trimming is accomplished so as to leave about 2 mm outside the reinforcement border, e.g., to facilitate suture attachment during surgery. In another embodiment, when shaping is desired, the reinforced reticulated elastomeric matrix can be trimmed along its length and/or width direction, along any other regular curved dimensions such as circle or ellipse or along any irregular shape.

For a device of this invention comprising a reinforced reticulated elastomeric matrix, the maximum dimension of any cross-section perpendicular to the device's thickness is from about 0.25 mm to about 100 mm in one embodiment. In another embodiment, the maximum thickness of the device is from about 0.25 mm to about 20 mm.

The composite surgical mesh can be made available in various sizes including, for example, 5 cm×10 cm and 12 cm×15 cm, with a nominal thickness of 2 mm.

In one embodiment, devices incorporating reinforcement into a reticulated elastomeric matrix will have at least one characteristic within the following ranges of performance. The suture pullout strength is from about 1.1 lbs/ft to about 17 lbs/ft (from about 5 Newtons to about 75 Newtons) in one embodiment or from about 2.3 lbs/ft to about 9.0 lbs/ft (from about 10 Newtons to about 40 Newtons) in another embodiment. The break strength is from about 9 lbs/ft to about 103 lbs/ft (from about 40 Newtons to about 450 Newtons) and from about 23 lbs/ft to about 68 lbs/ft (from about 100 Newtons to about 300 Newtons) in another embodiment. The ball burst strength is from about 34 lbsf to about 135 lbsf (from about 150 Newton to about 600 Newtons) in one embodiment or from about 45 lbsf to about 124 lbsf (from about 200 Newton to about 550 Newtons) in another embodiment.

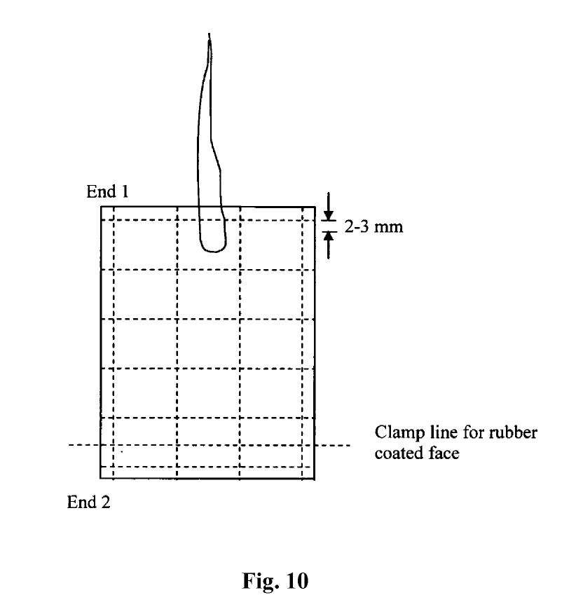

The suture pullout strength test was carried out using an INSTRON Tester (Model 3342) equipped with 1 kN pneumatic grips upper and lower gripping jaws, each having opposed 25 mm×25 mm rubber coated gripping faces. FIG. 10 illustrates the geometry of the reinforced specimen and the suture in an embodiment of the suture pullout strength test. The test suture wais a length of 2-0 ETHIBOND braided polyester suture. After the instrument's gauge length was set to 60 mm (2.36 inches), one end (End 2) of the reinforced reticulated elastomeric matrix device to be tested was clamped into the instrument's lower fixed jaw. The ETHIBOND test suture was inserted into the other end (End 1) of the reinforced reticulated elastomeric matrix device by using a needle. A loop was formed by the two ends of the test suture strands. The test suture was attached to the reinforced device 2 to 3 mm below the horizontal reinforcement line closest to the device's edge and, preferably, towards the center of the device's width, as illustrated in FIG. 10 for a device reinforced with a rectangular grid of fibers.

The free ends of the test suture were about 50 to 60 mm in length from the point where the test suture was attached to the reinforced reticulated elastomeric matrix device. The free ends of the suture were clamped into the instrument's upper movable jaw. Thereafter, the suture retention strength test was run at a rate of 100 mm/min (3.94 in/min) with the movable jaw moving upwards and away from the fixed jaw. The maximum force reached in the force-extension curve was noted as the suture retention strength, provided that the tear in the reinforced reticulated elastomeric matrix device was limited to the area near the End 1 horizontal grid line that was adjacent to the suture attachment position. The mean and standard deviation were determined from testing of a plurality of samples.

The break strength test was carried out in the same way as the suture pullout strength test described above except that the braided polyester suture is not used and the reinforced reticulated elastomeric matrix device to be tested was clamped between the instrument's lower fixed jaw and the upper movable jaw. Thereafter, the break strength test was run at a rate of 100 mm/min (3.94 in/min) with the movable jaw moving upwards and away from the fixed jaw. The maximum force reached in the force-extension curve was noted as the break strength.

The ball burst strength was measured pursuant to the test method described in ASTM Standard 3787 except that a ball with a diameter of 25.4 mm, and a crosshead speed of 102 mm/min (4 inch/min) were used.

In certain embodiments of the composite mesh device can range from about 0.5 mm to about 4 mm in thickness and may be in any two-dimensional or three-dimensional shape. Exemplary embodiments of a two-dimensional shape may include regular and irregular shapes, such as, for example, triangular, rectangular, circular, oval, elliptical, trapezoidal, pentagonal, hexagonal and irregular configurations, including one that corresponds to the shape of the defect, and other shapes. Examplary embodiments of a three-dimensional shape may include, plugs, cylinders, tubular structures, stent-like structures, and other configurations, including one that corresponds to the contours of the defect, and other configurations. The device may have a major axis having a length between about 2 cm to about 50 cm. The device may be in a square shape with a side having a length between about 2 cm to about 50 cm. Examples of specifications to be met by Biomerix biomaterial sheets used in the composite surgical mesh for embodiments of the invention are as follows:
Thickness 0.9±0.1 mm
Permeability >400 Darcy
Average cell size 250-500 μm
Density 3.6-3.9 lb/ft³
Break strength >30 psi
Elongation-to-break >150%
Polypropylene Mesh
   The polypropylene mesh employed for embodiments of the invention can be sourced, for example, from Biomedical Structures (Warwick, R.I.). The mesh can knitted using PPL100M-.004″ clear homopolymer polypropylene monofilament. The knitting process yields a flexible mesh with well defined apertures and can be cut into virtually any shape while retaining good edge integrity and fabric strength.
Mesh thickness 0.43±0.07 mm
2 bar diamond knitted construction
Largest pore size ˜1.4 mm×1.2 mm
Mesh density 50-58 g/m²
Break strength 226-325 N in machine direction and 155-232 N in counter-machine direction
Elongation-to-break 60-118% in machine direction and 90-164% in counter-machine direction

### Anti-Adhesion Coating

In one embodiment of the implantable device of the present invention, at last a portion of the outermost or macro surface is coated or fused to present a smaller macros surface area, because the internal surface area of pores below the surface becomes no longer accessible. Preferably, the implantable device is coated with a film comprising a biocompatible polymer. It is believed that the coated device would have a smoother outermost or macro surface as compared to a device having a fused outermost or macro surface. More preferably, the device may have at least a potion of the outermost or macro surface coated with a film comprising a biocompatible polymer. In another embodiment, the implantable device may a significant portion of the outermost or macro surface coated with a film of biocompatible polymer. In a specific embodiment, the implantable device may have all of the outermost or macro surface coated with a film of biocompatible polymer. Without being bound by any particular theory, it is thought that this decreased surface area provides more predictable and easier delivery and transport through long tortuous channels inside delivery-devices.

In another embodiment, the outermost or macro surface is coated or fused to alter “the porosity of the surface,” i.e., at least partially reduce the percentage of pores open to the surface, or limit or completely close-off pores of a coated or fused surface, i.e., that surface becomes nonporous because substantially no pores remain on the coated or fused surface. In one embodiment, the outermost or macro surface completely closes-off pores of a coated or fused surface, making it substantially or totally impermeable to liquid, such as body fluid, but still allows the internal interconnected and inter-communicating reticulated structure of the reticulated elastomeric matrix to remain open internally as well as on other outer or macro surfaces of the matrix that have not been coated or fused, e.g., the pores at a non-coated or non-fused portion of a matrix remain interconnected to other pores of the matrix, including that are within the matrix. These remaining open pores and/or surfaces can foster cellular ingrowth and proliferation. In a specific embodiment, a coated and an uncoated outermost or macro surface are orthogonal to each other. In another embodiment, a coated and uncoated outermost or macro surface are at an oblique angle to each other. In another embodiment, a coated and uncoated outermost or macro surface are adjacent. In another embodiment, a coated and uncoated outermost or macro surface are nonadjacent. In another embodiment, a coated and uncoated outermost or macro surface are in contact with each other. In another embodiment, a coated and uncoated outermost or macro surface are not in contact with each other.

In another embodiment, a support structure, such as a one-dimensional, two-dimensional, or three-dimensional reinforcement is between the surface coating or film of biocompatible polymer and the internal interconnected and inter-communicating reticulatd structure of elastomeric matrix 10 containing the uncoated surface. In another embodiment, there is one or two dimensional reinforcements between the surface coating or film of biocompatible polymer and the internal interconnected and inter-communicating reticulatd structure of elastomeric matrix 10 conatining the uncoated surface. In another embodiment, there reinforcement between the surface coating or film of biocompatible polymer and the internal interconnected and inter-communicating reticulatd structure of elastomeric matrix 10 conatining the uncoated surface and the reinforcement is a two-dimensional reinforcement, and the two-dimensional reinforcement may further comprise a grid of a plurality of one-dimensional reinforcement elements, wherein the one-dimensional reinforcement elements cross each other's paths. In further embodiments, the two-dimensional reinforcement may be a two-dimensional mesh made up of intersecting one-dimensional reinforcement elements. In one embodiment, the composite mesh comprising reticulated elastomeric matrix 10 is a multi-layered structure in which there is two dimensional reinforcements between the surface coating or film of biocompatible polymer and the internal interconnected and inter-communicating reticulatd structure of elastomeric matrix 10 conatining the uncoated surface. In another embodiment, the composite mesh comprising reticulated elastomeric matrix 10 is a multi-layered structure in which there is two dimensional reinforcements comprising a grid of a plurality of one-dimensional reinforcement elements between the surface coating or film of biocompatible polymer and the internal interconnected and inter-communicating reticulatd structure of elastomeric matrix 10 conatining the uncoated surface.

The 1-dimensional or 2-dimensional reinforcement or 3-dimensional for reinforcing the elastomeric matrix or to be placed or positioned or incorporated between surface coating or film of biocompatible polymer and the internal interconnected and inter-communicating reticulatd structure of elastomeric matrix 10.

In other applications, one or more planes of the macro surface of an implantable device made from reticulated elastomeric matrix 10 may be coated, fused or melted to improve its attachment efficiency to attaching means, e.g., anchors or sutures, so that the attaching means does not tear-through or pull-out from the implantable device. Without being bound by any particular theory, creation of additional contact anchoring macro surface(s) on the implantable device, as described above, is thought to inhibit tear-through or pull-out by providing fewer voids and greater resistance.

The fusion and/or selective melting of the macro surface layer of elastomeric matrix 10 can be brought about in several different ways. In one embodiment, a knife or a blade used to cut a block of elastomeric matrix 10 into sizes and shapes for making final implantable devices can be heated to an elevated temperature. In another embodiment, a device of desired shape and size is cut from a larger block of elastomeric matrix 10 by using a laser cutting device and, in the process, the surfaces that come into contact with the laser beam are fused. In another embodiment, a cold laser cutting device is used to cut a device of desired shape and size. In yet another embodiment, a heated mold can be used to impart the desired size and shape to the device by the process of heat compression. A slightly oversized elastomeric matrix 10, cut from a larger block, can be placed into a heated mold. The mold is closed over the cut piece to reduce its overall dimensions to the desired size and shape and fuse those surfaces in contact with the heated mold. In each of the aforementioned embodiments, the processing temperature for shaping and sizing is greater than about 15° C. in one embodiment. In another embodiment, the processing temperature for shaping and sizing is in excess of about 100° C. In another embodiment, the processing temperature for shaping and sizing is in excess of about 130° C. In another embodiment, the layer(s) and/or portions of the macro surface not being fused are protected from exposure by covering them during the fusing of the macro surface.

The coating on the macro surface or the film of biocompatible polymer can be made from a biocompatible polymer, which can include be both biodegradable or absorbable and non-biodegradable or non-absorbable polymers or permanent polymers. Suitable absorbable, biodegradable, non-biodegradable, non-absorbable polymers or permanent polymers include those biocompatible polymers disclosed in the section titled “Imparting Endopore Features”. Exemplary biodegradable polymers that can be used as coatings include but not limited to copolymers of caprolactone, lactic acid, glycolic acid, acid d-, l- and meso lactide and para-dioxanone, etc. or mixtures thereof. In another embodiemnt, biodegradable or bioabsorbable coatings made from copolymers of caprolactone with lactic acid, glycolic acid, acid d-, l- and meso lactide and para-dioxanone para-dioxanone are considered favorable for coating applications for providing anti-adhesion properties with copolymers of caprolactone with lactic acid in the the ratio of 40/60, 30/70 or 20/80 polycaprolactone to polylactic acid being prefrred for anti-adhesion properties. Further, the thermoplastic biodegradable or bioabsorbable polymer used for coating may comprise an ε-caprolactone copolymer, and optionally an ε-caprolactone-lactic acid copolymer or an ε-caprolactone-lactide copolymer. In another embodiment, biodurable or permanent biocompatible polymers further include polymers with relatively low chronic tissue response, such polyurerthane such as polycarbonate polyurethanes, polysiloxane polyurethanes, poly(siloxane-co-ether) polyurethanes, polycarbonate polysiloxane polyurethanes, polycarbonate urea-urethanes, polycarbonate polysiloxane urea-urethanes and the like and their mixtures. In another embodiment, biodurable or permanent biocompatible polymers include silicone. Biologically derived biomaterials are utilized as anti-adhesion coatings in other embodiments of the invention. Examples of suitable biologically derived biomaterials include reprocessed collagen, Hyaluronic acid (HA) or functionalized proteoglycans, and any of these combined with PEG. It is to be understood that that listing of materials is illustrative but not limiting.

In certain embodiments of the implantable device, the device is a composite of a reticulated elastomeric matrix and a mesh material having another functional element, which is a thin layer, coating or film of either a permanent polymer or biodegradable polymer. Preferably, the thin layer, coating or film of either a permanent polymer or biodegradable polymer is used to reduce the potential for biological adhesions. Notably, the thin layer, coating or film may act as or impart anti-adhesion properties to the implantable device and provide a beneficial effect in the repair of soft tissue defects, such as, for example in the treatment of hernias. An anti-adhesion coating or film is believed to be particularly important for the implantable device, because in anatomical sites, such as the abdominal wall, adhesions are likely to form between internal organ structures and any surface of the implantable device that is exposed to the biologic environment. In one preferred embodiment, the surface coating or film is flexible, which allows for ease of delivery of the implantable device through a trocar or endoscope. Moreover, a flexible surface coating or film allows the implantable device to be capable of confirming to the shape of a soft tissue site.

In additional embodiments, the implantable device comprising a reticulated resiliently compressible elastomeric matrix having a plurality of pores, and further comprising a one- or two-dimensional reinforcement, can further comprise a coating. The coating is important to impart anti-adhesion functionality, and is especially important in anatomic sites such as abdominal wall wherein adhesions are likely to form between internal organ structures and the exposed mesh surface. The combination of the antiadhesion coating and the Biomerix Biomaterial may result in less long term pain as current surgical mesh can allow for “scar” plate formation which is linked to chronic pain. The preferred anti-adhesion coating materials of a Purasorb PLC 7015 (Poly (L-lactide co ε-caprolactone) 70:30 molar ratio) provides an exceptionally flexible and durable coating potentially minimizing adhesions while biodegrading within a year. The coating can be a film-forming polymer, such as at least one silicone or at least one bioabsorbable polymer. Also, the coating may be applied as a solution in a solvent for the polymer, for example, with a polymer content in the coating solution of from about 1% to about 40% by weight. According to other embodiments, the coating solution may be applied by dip coating or spray coating the solution onto the reticulated elastomeric matrix, the solvent can be substantially or completely removed from the coating, and/or the solvent may be non-toxic and non-carcinogenic.

Other functional elements which may be incorporated with the reticulated elastomeric matrix to form a composite device include biologically derived collagen meshes (xenografts, allografts) used to enhance tissue response and minimize adhesion; polymeric and/or metallic structures used to impart shape memory; and markers including dyes used to differentiate between two sides of a mesh which may have differing characteristics. Any of these preferred functional elements may be incorporated with the biodurable reticulated elastomeric matrix using various processing techniques known in the art including adhesive bonding, melt processing, compression molding, suturing, and other techniques.

In other embodiments, a thermoplastic polymer is melted and applied to coat the reticulated elastomeric matrix, and optionally the thermoplastic polymer may be above a temperature of about 60° C. in its melted form. The melt can be applied by dip coating, extruding or coextruding the melt onto the reticulated elastomeric matrix. Further, the thermoplastic polymer may comprise an ε-caprolactone copolymer, and optionally an ε-caprolactone-lactic acid copolymer or an ε-caprolactone-lactide copolymer. In addition, the implantable device for such embodiments may be compressive molded and/or annealed after being reinforced.

In other embodiments, the coating is formed into a film, and is then bonded to the implantable device using an adhesive, such as Nusil™, Chronoflex™, or a biodegradable polymer.

In embodiments of the invention, an optional anti-adhesion coating can be added. The coating can consist of biodegradable or biodurable polymeric materials. For example, a polyurethane coating such as Chronoflex AR™ or expanded PTFE can be applied to a sheet of the Biomerix Biomaterial in the “sandwich” design, or optionally the construct can be made to eliminate one layer of Biomerix Biomaterial in an “open face sandwich design,” and the coating can be placed on the textile directly, with the advantage that large sizes of the device for embodiments of the invention can be delivered in a rolled form factor for laparoscopic surgery through standard sized trocar cannula (e.g., 12 mm to 18 mm).

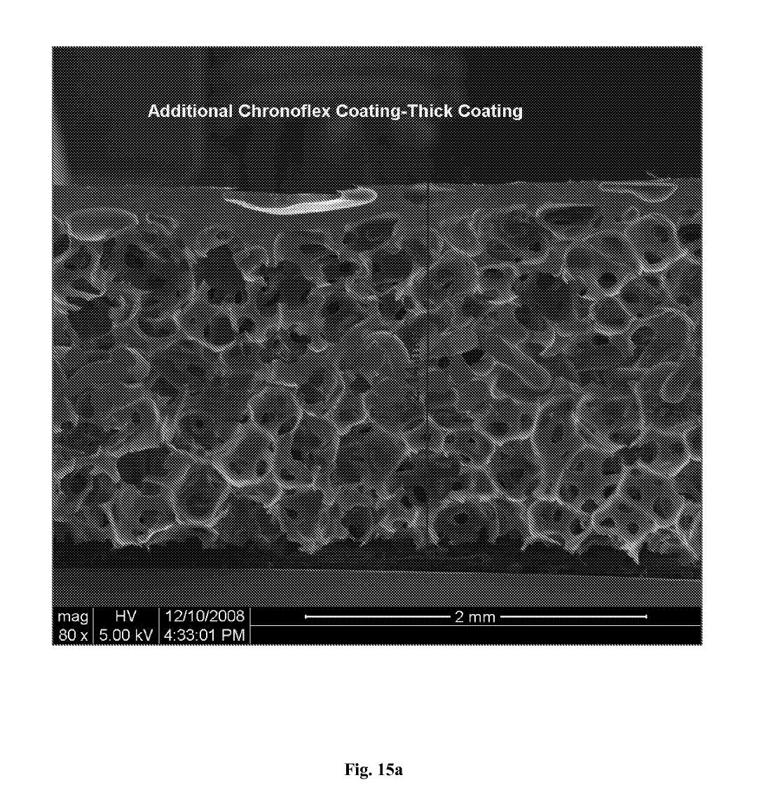

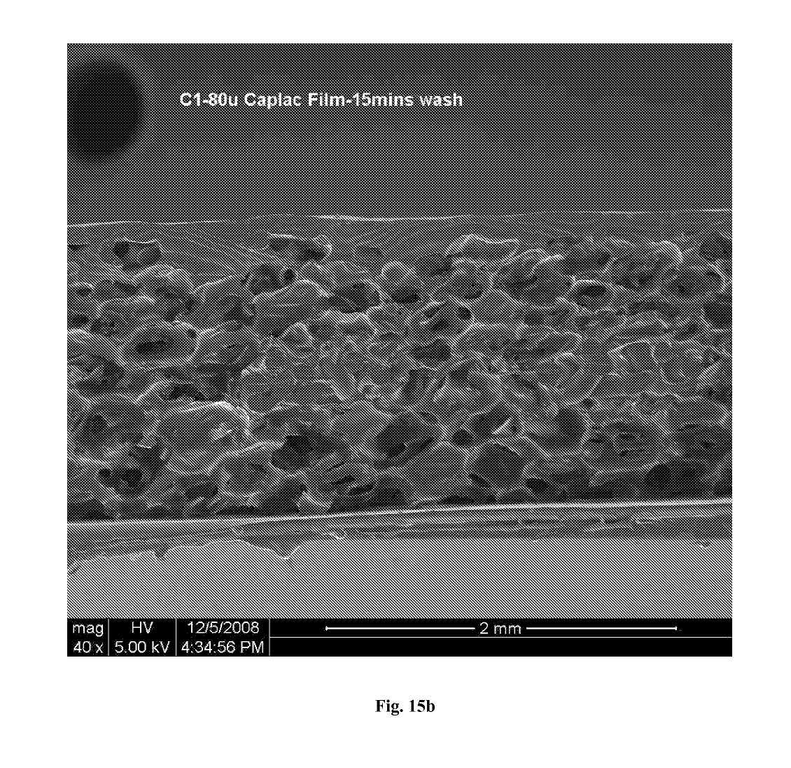

Another embodiment of the invention uses the composition described above with the use of biodegradable polymers or collagen. Examples of suitable biodegradable polymers are copolymers of Polylactide and Polycaprolactone, such as (Purasorb PLC 7015); co-polymers of Polyglycolide and Polycaprolactone; copolymers of Polyethylene Glycol (PEG) and polylactide and/or Polyglycolide; or mixtures of the polymers. The fabrication of such embodiment is similar to the foregoing example. However the films of the degradable polymer can be casted directly onto the medical textile and Biomerix matrix, or optionally the degradable polymer can be melt bonded onto the surface medical textile or the Biomerix Biomaterial. The bonded biodegradable film to the textile can be adhered to the Biomerix Biomaterial via adhesives such as Nusil™, Chronoflex AR™, or solutions of the degradable polymer. FIGS. 15a-15c illustrate examples of such an embodiment.

Biologically derived biomaterials may be utilized as anti-adhesion coatings in other embodiments of the invention. Examples of suitable biologically derived biomaterials include reprocessed collagen, Hyaluronic acid (HA) or functionalized proteoglycans, and any of these combined with PEG.

In one embodiment, the surface coating or film of biocompatible polymer is applied or incorporated on to a composite where the reinforcement is incorporated between two layers of the elastomeric matrix. In another embodiment, the surafce coating or film of biocompatible polymer is applied or incorporated on to a composite where the reinforcement is incorporated between two layers of the elastomeric matrix such as a sandwich design. The surace coating or film of biocompatible polymer is placed, attached, adhesive bonded, melt bonded to one of the two sides the reticulated elastomeric matrix that is being reinforced with one or two or three dimensional reinforcements. In another embodiment, the surace coating or film of biocompatible polymer is placed, attached, adhesive bonded, melt bonded to both sides the reticulated elastomeric matrix that is being reinforced with one or two or three dimennsional reinforcements.

In one embodiment, the surafce coating or film of biocompatible polymer is applied or incorporated on to a composite conatining multiple layers of reinforcement and elastomeric matrix can be stacked in an alternating fashion.

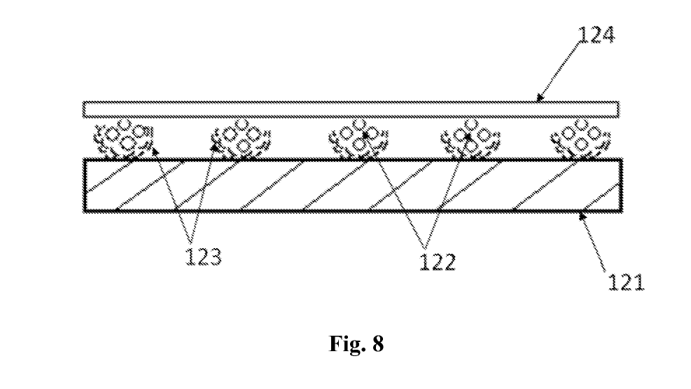

FIG. 8 shows a schematic of a coated composite where the 2-dimensional mesh reinforcement (122) is attached to one layer of elastomeric matrix (121) using an adhesive (123) and a film of biocompatible polymer (124) act as a coating. In one embodiment, the film of biocompatible polymer (124) act as an antiadhesive coating.

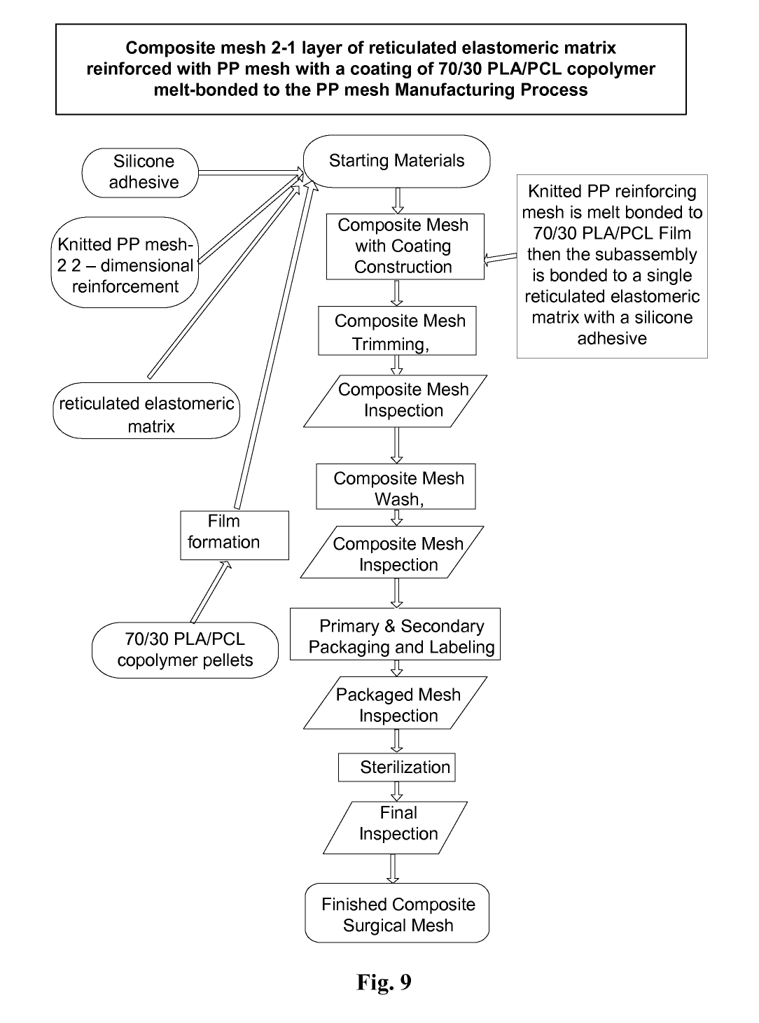

FIG. 9 shows a schematic of manufacturing of a coated composite where the 2-dimensional mesh reinforcement is attached to one layer of elastomeric matrix using an adhesive and a film of biocompatible polymer (124) act as an adhesive coating starting from initial raw materials to the finished product.

### Biologically Active Agent

In one embodiment, the implantable device and/or its reinforcement can be coated with one or more biologically active molecules, such as the proteins, collagens, elastin, entactin-1, fibrillin, fibronectin, cell adhesion molecules, matricellular proteins, cadherin, integrin, selectin, H-CAM superfamilies, and the like described in detail herein.

A further embodiment involves the addition of a biologically active (“bioactive”) agent to either enhance healing and/or to minimize infection. The bioactive agent can be added to the polymer film layer to facilitate controlled drug delivery. Examples of such bioactive agent are Matrix Metaloprotease inhibitors, such as zinc chelators (etridonate and EDTA, as examples) or molecules such as antibiotics, for example, doxycycline and tetracyclinecyclooxygenase-2 (COX-2) inhibitors, angiotensin-converting enzyme (ACE) inhibitors, glucocorticoids, beta blockers, nitric oxide synthase (NOS) inhibitors, antioxidants, non-steroidal anti-inflammatory drugs (NSAIDs) and cellular adhesion molecules (CAMs), and combinations of these. Of these molecules, doxycycline is deemed particularly suitable, as the molecule imparts antibiotic properties and also is known to inhibit MMPs such as MMP-2 and MMP-9. Local inhibition of MMPs is important as hernia formation is linked to an imbalance of MMP regulation, hence a combination of bioactive molecules to inhibit MMPs, a tissue scaffold to optimize cellular angiogenesis, and a bioactive to decrease the probability for infection. Thus, such embodiment represents a design that provides multiple solutions.

In another embodiment, a top coating can be used to coat the film layer or the reticulated elastomeric matrix for the delivery of a second bioactive agent. A layered coating comprising respective layers of fast- and slow-hydrolyzing polymer, can be used to stage release of the bioactive agent or to control release of different bioactive agents placed in the different layers. Polymer blends may also be used to control the release rate of different bioactive agents or to provide a desirable balance of coating characteristics (e.g., elasticity, toughness) and drug delivery characteristics (e.g., release profile). Polymers with differing solvent solubilities can be used to build up different polymer layers that may be used to deliver different bioactive agents or to control the release profile of bioactive agents. The amount of bioactive agent present depends upon the particular bioactive agent employed and medical condition being treated.

In one embodiment, the bioactive agent is present in an effective amount. In another embodiment, the amount of bioactive-agent represents from about 0.01% to about 60% of the coating by weight. In still another embodiment, the amount of bioactive agent represents from about 0.01% to about 40% of the coating by weight. In a further embodiment, the amount of bioactive agent represents from about 0.1% to about 20% of the coating by weight. Many different bioactive-agents can be used in conjunction with the reticulated elastomeric matrix or film used for anti-adhesion functionality.

In general, bioactive agents that may be administered via pharmaceutical compositions for embodiments of the invention include, without limitation, any therapeutic or pharmaceutically-active agent (including but not limited to nucleic acids, proteins, lipids, and carbohydrates) that possesses desirable physiologic characteristics for application to the implant site or administration via pharmaceutical compositions of the invention. Therapeutics include, without limitation, anti-infectives, such as antibiotics and antiviral agents; chemotherapeutic agents (e.g., anticancer agents); anti-rejection agents; analgesics and analgesic combinations; anti-inflammatory agents; hormones such as steroids; growth factors (including but not limited to cytokines, chemokines, and interleukins) and other naturally derived or genetically engineered proteins, polysaccharides, glycoproteins and lipoproteins.

Such growth factors are described in “The Cellular and Molecular Basis of Bone Formation and Repair” by Vicki Rosen and R. Scott Thies, published by R. G. Landes Company, hereby incorporated herein by reference. Additional therapeutics for embodiments of the invention include, for example, thrombin inhibitors, anti-thrombogenic agents, thrombolytic agents, fibrinolyticagents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti-secretory agents, actin inhibitors, remodeling inhibitors, antisense nucleotides, anti-metabolites, antiproliferatives, anticancer chemotherapeutic agents, anti-inflammatory steroids, non-steroidal anti-inflammatory agents, immunosuppressive agents, growth hormone antagonists, growth factors, dopamine agonists, radio-therapeutic agents, peptides, proteins, enzymes, extracellular matrix components, angiotensin-converting enzyme (ACE) inhibitors, free radical scavengers, chelators, antioxidants, anti-polymerases, antiviral agents, photodynamic therapy agents and gene therapy agents.

Additional therapeutics for embodiments of the invention include, for example, various proteins (including short chain peptides), growth agents, chemotatic agents, growth factor receptors. For example, in one embodiment, the pores of the reticulated elastomeric matrix may be partially or completely filled with biocompatible resorbable synthetic polymers or biopolymers (such as collagen or elastin), biocompatible ceramic materials (such as hydroxyapatite), and combinations thereof, and may optionally contain materials that promote tissue growth through the device, or alternatively these materials can be added to the anti-adhesion film. Such tissue-growth materials include, but are not limited to, autograft, allograft or xenograft bone, bone marrow and morphogenic proteins. Biopolymers can also be used as conductive or chemotactic materials, or as delivery vehicles for growth factors. Examples include recombinant collagen, animal-derived collagen, elastin and hyaluronic acid.

According to embodiments of the invention, surface treatments can also be present on the surface of the materials. For example, bioactive peptide sequences (RGD's) could be attached to the surface to facilitate protein adsorption and subsequent cell tissue attachment. Bioactive molecules include, without limitation, proteins, collagens (including types IV and XVIII), fibrillarcollagens (including types I, II, III, V, XI), FACIT collagens (types IX, XII, XIV), other collagens (types VI, VII, XIII), short chain collagens (types VIII, X), elastin, entactin-I, fibrillin, fibronectin, fibrin, fibrinogen, fibroglycan, fibromodulin, fibulin, glypican, vitronectin, laminin, nidogen, matrilin, perlecan, heparin, heparan sulfate proteoglycans, decorin, filaggrin, keratin, syndecan, agrin, integrins, aggrecan, biglycan, bone sialoprotein, cartilage matrix protein, Cat-301 proteoglycan, CD44, cholinesterase, HB-GAM, hyaluronan, hyaluronan binding proteins, mucins, osteopon tin, plasminogen, plasminogen activator inhibitors, restrictin, serglycin, tenascin, thrombospondin, tissue-type plasminogenactivator, urokinase type plasminogen activator, versican, von Willebrand factor, dextran, arabinogalactan, chitosan, polyactide-glycolide, alginates, pullulan, and gelatin and albumin.

In embodiments of the invention, additional bioactive molecules include, without limitation, cell adhesion molecules and matricellular proteins, including those of the immunoglobulin (e.g., including monoclonal and polyclonal antibodies), cadherin, integrin, selectin, and H-CAM superfamilies. Examples include, without limitation, AMOG, CD2, CD4, CD8, C-CAM (CELL-CAM 105), cell surface galactosyltransferase, connexins, desmocollins, desmoglein, fasciclins, F11, GP Ib-IXcomplex, intercellular adhesion molecules, leukocyte common antigen protein tyrosine phosphate (LCA, CD45), LFA1, LFA-3, mannose binding proteins (MBP), MTJCI8, myelin associated glycoprotein (MAG), neural cell adhesion molecule (NCAM), neurofascin, neruoglian (or neuroglian), neurotactin, netrin, PECAM-1, PH-20, semaphorin, TAG-I, VCAM-1, SPARClosteonectin, CCNI (CYR61), CCN2 (CTGF; Connective Tissue Growth Factor), CCN3 (NOV), CCN4 (WISP-I), CCN5 (WISP-2), CCN6 (WISP-3), occludin and claudin.

Growth factors employed in embodiments of the invention include, without limitation, BMP's (1-7), BMP-like Proteins (GFD-5, -7, -8), epidermal growth factor (EGF), erythropoietin (EPa), fibroblast growth factor (FGF), growth hormone (GH), growth hormone releasing factor (GHRF), granulocyte colony-stimulating factor(G-CSF), granulocyte-macrophage colony-stimulating factor (GM-CSF), insulin, insulin-like growth factors (IGF-I,IGF-II), insulin-like growth factor binding proteins (IGFBP), macrophage colony-stimulating factor (M-CSF), Multi-CSF (II-3), platelet-derived growth factor (PDGF), tumor growth factors (TGF-alpha, TGF-beta), tumor necrosis factor (TNF-alpha), vascular endothelial growth factors (VEGF's), angio proietins, placenta growth factor (PIGF), interleukins, and receptor proteins or other molecules that are known to bind with the aforementioned factors. Short chain peptides include, without limitation (designated by single letter amino acid code), RGD, EILDY, RGDS, RGES, RFDS, GRDGS, GRGS, GRGDTP and QPPRARI.

### Method of Making Composite

Methods of producing the device for embodiments of the invention begin, for example, with production of a block of polyurethane matrix. The block of polyurethane foam is machined into thin slices; adhesive is applied to the polypropylene knitted mesh in a controlled manner; the composite mesh is assembled in a tri-layer structure; and the layers are cured. Individual implants are trimmed to size. The entire mesh is then washed to remove any unreacted processing aids or other impurities.

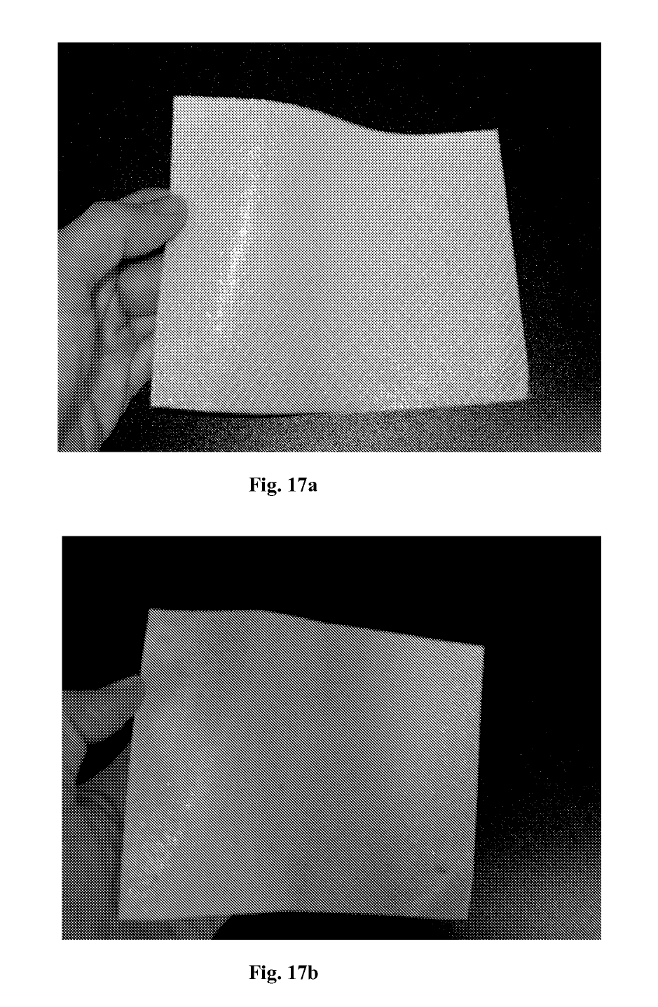

In additional embodiments, bonding of the different materials can be done using multiple approaches. One suitable process for embodiments of the invention is to bond a medical textile to the Biomerix matrix with Nusil™, Chronoflex™, or a biodegradable polymer and subsequently bonding this construct with a casted film of Chronoflex AR™ to a target thickness of about 20 to about 200 μm with the same adhesives listed beforehand. FIGS. 15a-15c, 17a and 17b illustrate examples of such an embodiment.

An example of a suitable adhesive used to bond the substrates for embodiments of the invention is silicone adhesive (NuSil™ MED2-4213).

In embodiments of the invention, the Biomerix biomaterial composite surgical mesh is made from a biostable, cross-linked, reticulated (possessing interconnected and intercommunicating open pores), resilient elastomeric matrix made from polycarbonate polyurethane-urea (Biomerix biomaterial). For example, a polypropylene mesh (knitted polypropylene monofilament fibers, Biomedical Structure PPM-5) is sandwiched between the two layers, and silicone adhesive (NuSil™ MED2-4213) is used to bond the substrates.

The incorporation of the reinforcement into the matrix can be achieved by various ways, including but not limited use of an adhesive such as silicone, polyurethanes, biodegradable polymers, permanent polymers. Exemplary polyurethane that can be used as adhesives include not limited to polycarbonate polyurethanes, polysiloxane polyurethanes, poly(siloxane-co-ether) polyurethanes, polycarbonate polysiloxane polyurethanes, polycarbonate urea-urethanes, polycarbonate polysiloxane urea-urethanes and the like and their mixtures. Exemplary biodegradable polymers that can be used as adhesives include not limited to copolymers of caprolactone, lactic acid, glycolic acid, acid d-, l- and meso lactide and para-dioxanone, etc. or mixtures thereof. In another embodiment, biodegradable polymers that can be used as adhesives comprise copolymers of caprolactone with lactic acid, glycolic acid, acid d-, l- and meso lactide and para-dioxanone, etc. or mixtures thereof.

The adhesive can be applied between the reinforcement and elastomeric matrix and cured. In another embodiment, the adhesive can be applied either to reinforcement or the elastomeric matrix or both before being cured. The adhesive can be applied by dip or spray coating, painted with a brush, by use of customized coating fixtures that can lay down or deliver a thin layer of adhesive using blades with adjustable heights followed by transfer of the thin layer of adhesive on to the reinforcement or the elastomeric matrix. Or both. In one embodiment, the adhesive is a solution and the polymer content in the adhesive solution is from about 1% to about 40% by weight. In another embodiment, the polymer content in the adhesive solution is from about 1% to about 20% by weight. In another embodiment, the polymer content in the adhesive solution is from about 1% to about 10% by weight. In one embodiment, the adhesive does not contain any solvents. The solvent or solvent blend for the coating solution is chosen, e.g., based on the considerations discussed in the previous section (i.e., in the “Imparting Endopore Features” section). In one embodiment, the adhesive can be cured between 50° C. and 150° C. and in another embodiment between 60° C. and 120° C. In one embodiment, the adhesive can be cured between 10 minutes and 3 hours and in another embodiment between 15 minutes and 2 hours.

The adhesive can be applied between the reinforcement and elastomeric matrix by melt-bonding the adhesive the reinforcement and elastomeric matrix. In another embodiment, the adhesive can be applied either to reinforcement or the elastomeric matrix. In another embodiment, the adhesive may be applied by melting the film-forming adhesive polymer and applying the melted polymer through a die, in a process such as extrusion or coextrusion, as a thin layer of melted. In these embodiments, the melted polymer either coats the reinforcement or coats the elastomeric matrix macro surface but does not penetrate into the interior to any significant depth or bridges or plugs pores of that surface. Thus, the reticulated nature of portions of the elastomeric matrix removed from the macro surface, and portions of the elastomeric matrix's macro surface not in contact with the melted polymer, is maintained. Upon applying pressure to create contact between elastomeric matrix and reinforcement, cooling and solidifying, the melted polymer forms a layer of solid coating on the elastomeric matrix and the reinforcement and in the interface between them. In one embodiment, the processing temperature of the melted thermoplastic adhesive polymer is at least about 60° C. In another embodiment, the processing temperature of the melted thermoplastic adhesive polymer is at least above about 90° C. In another embodiment, the processing temperature of the melted thermoplastic adhesive polymer is at least above about 120° C. The melt can be applied by extruding or coextruding or injection molding or compression molding or compressive molding the melt onto the reticulated elastomeric matrix.

FIG. 4 shows a schematic of manufacturing a “sandwich design” or a composite where the 2-dimensional mesh reinforcement is attached to two layers of elastomeric matrix using an adhesive starting from initial raw materials to the finished product.

Without being bound by any particular theory, too little adhesive may prevent adequate bonding while too much adhesive may lad to partial or full clogging of the pores of the reticulated elastomeric matrix. Too much adhesive can also lead to loss of flexibility during delivery and placement and a stiffer implant that may not be desirable. The coat weight of the adhesives can vary from 2 milligram/cm² to 35 milligram/cm² and in another embodiment, the coat weight of the adhesives can vary from 3.5 milligram/cm² to 25 milligram/cm².

In one embodiment, the incorporation of the reinforcement into the matrix can be achieved by various ways, including but not limited to stitching, sewing, weaving and knitting. In one embodiment, the attachment of the reinforcement to the matrix can be through a sewing stitch. In another embodiment, the attachment of the reinforcement to the matrix can be through a sewing stitch that includes an interlocking feature. In another embodiment, the incorporation of the reinforcement into the matrix can be achieved by foaming of the elastomeric matrix ingredients around a pre-fabricated or pre-formed reinforcement element made from a reinforcement and reticulating the composite structure thus-formed to create an intercommunicating and interconnected pore structure. In one embodiment, the reinforcement used does not interfere with the matrix's capacity to accommodate tissue ingrowth and proliferation. In an embodiment where sewing is used to incorporate the reinforcement into the matrix, the pitch of the stitch, i.e., the distance between successive stitches or attachment points within the same line, is from about 0.25 mm to about 4 mm in one embodiment or from about 1 mm to about 3 mm in another embodiment.

The coating or the film coating on elastomeric matrix 10 can be applied to the elastomeric matrix or to the reinforcements by use of an adhesive or bonding material that can be applied in various fashion such as by, e.g., dipping or spraying a coating solution comprising a polymer or a polymer and in embodiment that solution can be admixed with a pharmaceutically-active agent. In one embodiment, the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight. In another embodiment, the polymer content in the coating solution is from about 1% to about 10% by weight. In another embodiment, the coating may be applied as a solution in a solvent for the polymer, for example, with a polymer content in the coating solution of from about 1% to about 40% by weight. According to other embodiments, the coating solution may be applied by dip coating or spray coating the solution onto the reticulated elastomeric matrix, the solvent can be substantially or completely removed from the coating, and/or the solvent may be non-toxic and non-carcinogenic. In another embodiment, the layer(s) and/or portions of the macro surface not being solution-coated are protected from exposure by covering them during the solution-coating of the macro surface. The solvent or solvent blend for the coating solution is chosen, e.g., based on the considerations discussed in the previous section (i.e., in the “Imparting Endopore Features” section). In one embodiment, the coating or bonding material can be cured between 50° C. and 150° C. and in another embodiment between 60° C. and 120° C. In one embodiment, the adhesive or bonding material can be cured between 10 minutes and 3 hours and in another embodiment between 15 minutes and 2 hours.

In one embodiment, the coating on elastomeric matrix 10 may be applied by melting a film-forming coating polymer and applying the melted polymer onto the elastomeric matrix 10. In another embodiment, the film-forming coating polymer is a thermoplastic polymer that is melted, enters the pores 20 of the elastomeric matrix 10 or composite mesh comprising reticulated elastomeric matrix 10 and, upon cooling or solidifying, forms a coating on at least a portion of the solid material 12 of the elastomeric matrix 10. In other embodiments, a thermoplastic polymer is melted and applied to coat the reticulated elastomeric matrix. In another embodiment, the coating on elastomeric matrix 10 may be applied by melting the film-forming coating polymer and applying the melted polymer through a die, in a process such as extrusion or coextrusion, as a thin layer of melted polymer onto a mandrel formed by elastomeric matrix 10. In either of these embodiments, the melted polymer coats the macro surface and bridges or plugs pores of that surface but does not penetrate into the interior to any significant depth. Without being bound by any particular theory, this is thought to be due to the high viscosity of the melted polymer. Thus, the reticulated nature of portions of the elastomeric matrix removed from the macro surface, and portions of the elastomeric matrix's macro surface not in contact with the melted polymer, is maintained. Upon cooling and solidifying, the melted polymer forms a layer of solid coating on the elastomeric matrix 10. In one embodiment, the processing temperature of the melted thermoplastic coating polymer is at least about 60° C. In another embodiment, the processing temperature of the melted thermoplastic coating polymer is at least above about 90° C. In another embodiment, the processing temperature of the melted thermoplastic coating polymer is at least above about 120° C. The melt can be applied by extruding or coextruding or injection molding or compression molding or compressive molding the melt onto the reticulated elastomeric matrix. In another embodiment, the layer(s) and/or portions of the macro surface not being melt-coated are protected from exposure by covering them during the melt-coating of the macro surface.

In one embodiments, the film of biocompatible polymer that is to be used as coating is first formed by extrusion, injection molding compression molding or solvent casting. The film of biocompatible polymer is then bonded to the implantable device using an adhesive. The adhesive can be applied between the reinforcement and elastomeric matrix and cured. In another embodiment, the adhesive can be applied either to reinforcement or the elastomeric matrix or both before being cured. The adhesive can be applied by dip or spray coating, painted with a brush, by use of customized coating fixtures that can lay down or deliver a thin layer of adhesive using blades with adjustable heights followed by transfer of the thin layer of adhesive on to the reinforcement or the elastomeric matrix or both. In one embodiment, the the film of biocompatible polymer is bonded by an adhesive applied by dip coating. Exemplary adhesives include but not limited to Nusil™, Chronoflex™, Elast-Eon™ or a biodegradable polymer.

In one embodiments, the film of biocompatible polymer that is to be used as coating is first formed by extrusion, injection molding compression molding or solvent casting.

In another embodiment of the composite mesh comprising reticulated elastomeric matrix 10, the film of biocompatible polymer is first melt bonded to the one or two dimensional reinforcements which in turn is then bonded to reticulated elastomeric matrix the using an adhesive. In another embodiment of the composite mesh comprising reticulated elastomeric matrix 10, the film of biocompatible polymer is first melt bonded to one side of the one or two dimensional reinforcements whose other side in turn is then bonded to reticulated elastomeric matrix the using an adhesive. In another embodiment of the composite mesh comprising reticulated elastomeric matrix 10, the film of biocompatible polymer is first melt bonded to the one or two dimensional reinforcements which in turn is then bonded to reticulated elastomeric matrix containing the uncoated surface the using an adhesive. In another embodiment of the composite mesh comprising reticulated elastomeric matrix 10, the film of biocompatible polymer is first melt bonded to the one or two dimensional reinforcements which in turn is then bonded to reticulated elastomeric matrix surface the using an adhesive. Exemplary adhesives include but not limited to Nusil™, Chronoflex™, Elast-Eon™ or a biodegradable polymer. Other adhesives are discussed and described in one the previous section titled “Reinforcement Incorporation”

In another embodiment of the composite mesh comprising reticulated elastomeric matrix 10, the film of biocompatible polymer is first melt bonded to the one or two dimensional reinforcements which in turn is then again melt bonded to reticulated elastomeric matrix. In another embodiment of the composite mesh comprising reticulated elastomeric matrix 10, the film of biocompatible polymer is first melt bonded to one side of the one or two dimensional reinforcements whose other side in turn is then again melt bonded to reticulated elastomeric matrix. In another embodiment of the composite mesh comprising reticulated elastomeric matrix 10, the film of biocompatible polymer is first melt bonded to the one or two dimensional reinforcements which in turn is then again melt bonded to reticulated elastomeric matrix containing the uncoated surface. In another embodiment of the composite mesh comprising reticulated elastomeric matrix 10, the film of biocompatible polymer is first melt bonded to the one or two dimensional reinforcements which in turn is then again melt bonded to reticulated elastomeric matrix surface. The melt bonding can take place by either melting or partially melting the film of biocompatible polymer. In another embodiment, the melt bonding can take place by either melting or partially melting the a second film forming biocompatible coating polymer that can can include be both biodegradable or absorbable and non-biodegradable or non-absorbable polymers or permanent polymers. In one embodiment, the melt bonding processing temperature is at least about 60° C. In another embodiment, the melt bonding processing temperature is at least about 90° C. In another embodiment, the melt bonding processing temperature is at least about 120° C.

The thickness of the surface coating or the thickness of the film of biocompatible polymer that is to be used as coating varies between 30 and 250 microns in one embodiment. In another embodiment, the thickness of the surface coating or the thickness of the film of biocompatible polymer that is to be used as coating varies between 60 and 175 microns. In another embodiment, the thickness of the surface coating or the thickness of the film of biocompatible polymer that is to be used as coating varies between 80 and 140 microns. While a thicker coating or film thickness can provide better bonding with the reinforcements or the device or the reticulated elastomeric matrix, it may also lead to loss in flexibility, difficulty in delivery.

### Compressive Molding

In certain embodiments, the implantable device of the present invention may be compressive molded or annealed. Additionally, the implantable device for such embodiments can be compressive molded after being reinforced and/or annealed after being reinforced. Further, the implantable device for such embodiments may be compressive molded and/or annealed after being reinforced.

In one embodiment, the implantable device may be compressive molded by applying a pressure to decrease the volume of the implantable device. For example, the implantable device may be compressed in at least one dimension, e.g., 1-dimensional compression, 2-dimensional compression, or 3-dimensional compression, in a compressive molding process. In certain embodiments, the reticulated elastomeric matrix is compressed before being attached to a support structure. In such embodiments, the matrix remains compressed during the inclusion of the reinforcement.

In one embodiment, the implantable device is made from a matrix that is oriented in one dimension. In another embodiment, the implantable device is made from a matrix that is oriented in two dimensions. In another embodiment, the implantable device is made from a matrix that is oriented in three dimensions. In another embodiment, there is substantially no preferred orientation in the matrix. In another embodiment, the matrix orientation occurs during initial foam formation. In another embodiment, the matrix orientation occurs during reticulation. In another embodiment, the matrix orientation occurs during any secondary processing, such as by compressive molding, that may occur subsequent to reticulation. The results of orientation are manifested by enhanced properties and/or enhanced performance in the direction of orientation. For example, tensile properties, such as tensile strength, can be enhanced in the foam rise direction while only a slight change or no significant change in tensile strength occurs in the directions orthogonal to the foam rise direction.

In one secondary processing method, referred to herein as compressive molding, desirable enhanced performance is obtained by densification and/or orientation in one dimension, two dimensions or three dimensions using different temperatures. In one embodiment, the densification and/or orientation can be effected without the use of a mold. In another embodiment, the densification and/or orientation is facilitated by using a mold. As discussed below, the densification and/or orientation is usually carried out at a temperature above 25° C., e.g., from about 105° C. to about 180° C., over a period of time where the length of time depends on the temperature(s) used. In another embodiment, the compressive molding process is conducted in a batch process. In another embodiment, the compressive molding process is conducted in a continuous process.

A “preform” is a shaped uncompressed reticulated elastomeric matrix that has been cut or machined from a block of reticulated elastomeric matrix for use in secondary processing, such as compressive molding. The preform can have a predetermined size and shape. In one embodiment, the size and shape of the preform is determined by the final or desired compression ratio that will be imparted during compressive molding.

When a mold is used, the mold cavity can have fixed shape, such as a cylinder, cube, sphere or ellipsoid, or it can have an irregular shape. The reticulated elastomeric matrix, upon being compressive molded, conforms to a great degree to the geometry of the mold at the end of the densification and/or orientation step.

If the reduction in the dimension that is reduced during compressive molding is expressed in terms of linear compressive strain, i.e., the change in a dimension over that original dimension, the linear compressive strain is from about 3% to about 97%. In another embodiment, the linear compressive strain is from about 15% to about 95%. In another embodiment, the linear compressive strain is from about 25% to about 90%. In another embodiment, the linear compressive strain is from about 30% to about 85%. In another embodiment, the linear compressive strain is from about 40% to about 75%.

In another embodiment, during compressive molding the radius dimension of a cylindrical preform is reduced, i.e., the circumference is reduced, such that the dimensional reduction occurs in two directions, while, in the other direction, the cylinder's height remains substantially unchanged. In another embodiment, during compressive molding the radius dimension of a cylindrical preform is reduced, while, in the other direction, the cylinder's height remains unchanged.

Compressive molding of the reticulated elastomeric matrix may be conducted at temperatures above 25° C. and can be carried out from about 100° C. to about 190° C. in one embodiment, from about 110° C. to about 180° C. in another embodiment, or from about 120° C. to about 145° C. in another embodiment. In another embodiment, as the temperature at which the compressive molding process is carried out increases, the time at which the compressive molding process is carried out decreases. The time for compressive molding is usually from about 10 seconds to about 10 hours. In another embodiment, the compressive molding time is from about 30 seconds to about 5 hours. In another embodiment, the compressive molding time is from about 30 seconds to about 3 hours. As the temperature at which the compressive molding process is conducted is raised, the time for compressive molding decreases. At higher temperatures, the time for compressive molding must be short, as a long compressive molding time may cause the reticulated elastomeric matrix to thermally degrade. For example, in one embodiment, at temperatures of about 160° C. or greater, the time for compressive molding is about 30 minutes or less in one embodiment, about 10 minutes or less in another embodiment, or about 5 minutes or less in another embodiment. In another embodiment, at a temperature of about 150° C., e.g., from about 145° C. to about 155° C., the time for compressive molding is about 60 minutes or less in one embodiment, about 20 minutes or less in another embodiment, or about 10 minutes or less in another embodiment. In another embodiment, at temperatures of about 130° C., e.g., from about 125° C. to about 135° C., the time for compressive molding is about 240 minutes or less in one embodiment, about 120 minutes or less in another embodiment, or about 30 minutes or less in another embodiment.

According to embodiments of the invention, the Biomerix biomaterial composite surgical mesh is provided sterile for single use. Each mesh implant for embodiments of the invention can be packaged separately and provided sterile for single use. Devices for embodiments of the invention can be sealed in a pouch, such as a single Tyvek™ pouch (1073B Tyvek™ sealed to 48 PET/200 LDPE). Multiple implants, such as three implants, can be packaged in a single carton.

Other Post-Processing of the Reticulated Elastomeric Matrix or Composite Mesh

Elastomeric matrix 10 or composite mesh comprising reticulated elastomeric matrix can undergo a further processing step or steps, in addition to those already discussed above. For example, elastomeric matrix 10 or the products made from elastomeric matrix 10 can be annealed to stabilize the structure.

In one embodiment, annealing at elevated temperatures can promote increased crystallinity in polyurethanes. In another embodiment, annealing at elevated temperatures can also promote structural stabilization in cross-linked polyurethanes and long-term shelf-life stability. The structural stabilization and/or additional crystallinity can provide enhanced shelf-life stability to implantable-devices made from elastomeric matrix 10. In one embodiment, without being bound by any particular theory, annealing leads to relaxation of the stresses formed in the reticulated elastomeric matrix structure during foam formation and/or reticulation.

In one embodiment, annealing is carried out at temperatures in excess of about 50° C. In another embodiment, annealing is carried out at temperatures in excess of about 100° C. In another embodiment, annealing is carried out at temperatures in excess of about 125° C. In another embodiment, annealing is carried out at temperatures of from about 100° C. to about 135° C. In another embodiment, annealing is carried out at temperatures of from about 100° C. to about 130° C. In another embodiment, annealing is carried out at temperatures of from about 100° C. to about 120° C. In another embodiment, annealing is carried out at temperatures of from about 105° C. to about 115° C.

In another embodiment, annealing is carried out for at least about 2 hours. In another embodiment, annealing is carried out for from about 2 to about 15 hours. In another embodiment, annealing is carried out for from about 3 to about 10 hours. In another embodiment, annealing is carried out for from about 4 to about 8 hours.

Annealing can be carried out with or without constraining the device. In another embodiment, the elastomeric matrix 10 is geometrically unconstrained while it is annealed, e.g., the elastomeric matrix is not surrounded by a mold. In another embodiment, the elastomeric matrix 10 is geometrically constrained while it is annealed, e.g., the elastomeric matrix is constrained by a surface, such as a mold surface, on one or more sides so that its dimension(s), such as its thickness, does not change substantially during annealing. In this embodiment, the elastomeric matrix 10 is not compressed to any significant extent by its constraint, thus, such annealing differs from compressive molding in this respect.

In one embodiment, compressive molding can be optionally followed by further annealing of the (already) compressed reticulated elastomeric matrix at a temperature of from about 110° C. to about 140° C. and for a time period of from about 15 minutes to about 4 hours. As with compressive molding, annealing can be carried while restraining the compressed matrix in a mold or without a mold. In another embodiment, annealing can be carried while restraining the compressed matrix in a mold. If the initial compressive molding occurred at a temperature or about 150° C. or greater, the time for annealing should be short so as to avoid potential for thermal degradation of the compressed reticulated elastomeric matrix at long annealing times. For example, compressive molding at a temperature of about 150° C. or greater can be followed by annealing of the compressed reticulated elastomeric matrix at a temperature of from about 125° C. to about 135° C. for a time period of from about 30 minutes to about 3 hours.

Elastomeric matrix 10 composite mesh comprising reticulated elastomeric matrix may be molded into any of a wide variety of shapes and sizes during its formation or production. The shape may be a working configuration, such as any of the shapes and configurations described in the applications to which priority is claimed, or the shape may be for bulk stock. Stock items may subsequently be cut, trimmed, punched or otherwise shaped for end use. The sizing and shaping can be carried out by using a blade, punch, drill or laser, for example. In each of these embodiments, the processing temperature or temperatures of the cutting tools for shaping and sizing can be greater than about 100° C. In another embodiment, the processing temperature(s) of the cutting tools for shaping and sizing can be greater than about 130° C. Finishing steps can include, in one embodiment, trimming of macrostructural surface protrusions, such as struts or the like, which can irritate biological tissues. In another embodiment, finishing steps can include heat annealing. Annealing can be carried out before or after final cutting and shaping.

Shaping and sizing can include custom shaping and sizing to match an implantable device to a specific treatment site in a specific patient, as determined by imaging or other techniques known to those in the art. In particular, one or a small number, e.g. less than about 6 in one embodiment and less than about 2 in another embodiment, of elastomeric matrices 10 can comprise an implantable device system for treating damaged tissue requiring repair and/or regeneration.

The dimensions of the shaped and sized devices made from elastomeric matrix 10 can vary depending on the particular tissue repair and regeneration site treated. In one embodiment, the major dimension of a device prior to being compressed and delivered is from about 0.5 mm to about 500 mm. In another embodiment, the major dimension of a device prior to being compressed and delivered is from about 10 mm to about 500 mm. In another embodiment, the major dimension of a device prior to being compressed and delivered is from about 50 mm to about 200 mm. In another embodiment, the major dimension of a device prior to being compressed and delivered is from about 30 mm to about 100 mm. Elastomeric matrix 10 can exhibit compression set upon being compressed and transported through a delivery-device, e.g., a catheter, syringe or endoscope. In another embodiment, compression set and its standard deviation are taken into consideration when designing the pre-compression dimensions of the device.

Biodurable reticulated elastomeric matrices 10, or composite mesh comprising reticulated elastomeric matrix or an implantable device system comprising such matrices, can be sterilized by any method known to the art including gamma irradiation, autoclaving, ethylene oxide sterilization, infrared irradiation and electron beam irradiation. In one embodiment, biodurable elastomers used to fabricate elastomeric matrix 10 tolerate such sterilization without loss of useful physical and mechanical properties. The use of gamma irradiation can potentially provide additional cross-linking to enhance the performance of the device.

In one embodiment, the sterilized products may be packaged in sterile packages of paper, polymer or other suitable material. In another embodiment, within such packages, elastomeric matrix 10 composite mesh comprising reticulated elastomeric matrix is compressed within a retaining member to facilitate its loading into a delivery-device, such as a catheter or endoscope, in a compressed configuration. In another embodiment, elastomeric matrix 10 comprises an elastomer with a compression set enabling it to expand to a substantial proportion of its pre-compressed volume, e.g., at 25° C., to at least 50% of its pre-compressed volume. In another embodiment, expansion occurs after elastomeric matrix 10 remains compressed in such a package for typical commercial storage and distribution times, which will commonly exceed 3 months and may be up to 1 or 5 years from manufacture to use.

Radio-Opacity

In one embodiment, implantable device can be rendered radio-opaque to facilitate in vivo imaging, for example, by adhering to, covalently bonding to and/or incorporating into the elastomeric matrix itself particles of a radio-opaque material. Radio-opaque materials include titanium, tantalum, tungsten, barium sulfate or other suitable material known to those skilled in the art.

Tissue Culture

The implantable device of the present invention may support cell types including cells secreting structural proteins and cells that produce proteins characterizing organ function. The ability of the elastomeric matrix to facilitate the co-existence of multiple cell types together and its ability to support protein secreting cells demonstrates the applicability of the elastomeric matrix in organ growth in vitro or in vivo and in organ reconstruction. In addition, the biodurable reticulated elastomeric matrix may also be used in the scale up of human cell lines for implantation to the body for many applications including implantation of fibroblasts, chondrocytes, osteoblasts, osteoclasts, osteocytes, synovial cells, bone marrow stromal cells, stem cells, fibrocartilage cells, endothelial cells, smooth muscle cells, adipocytes, cardiomyocytes, myocytes, keratinocytes, hepatocytes, leukocytes, macrophages, endocrine cells, genitourinary cells, lymphatic vessel cells, pancreatic islet cells, muscle cells, intestinal cells, kidney cells, blood vessel cells, thyroid cells, parathyroid cells, cells of the adrenal-hypothalamic pituitary axis, bile duct cells, ovarian or testicular cells, salivary secretory cells, renal cells, epithelial cells, nerve cells, stem cells, progenitor cells, myoblasts and intestinal cells.

The approach to engineer new tissue can be obtained through implantation of cells seeded in elastomeric matrices (either prior to or concurrent to or subsequent to implantation). In this case, the elastomeric matrices may be configured either in a closed manner to protect the implanted cells from the body's immune system, or in an open manner so that the new cells can be incorporated into the body. Thus in another embodiment, the cells may be incorporated, i.e. cultured and proliferated, onto the elastomeric matrix prior, concurrent or subsequent to implantation of the elastomeric matrix in the patient.

In one embodiment, the implantable device made from biodurable reticulated elastomeric matrix can be seeded with a type of cell and cultured before being inserted into the patient, optionally using a delivery-device, for the explicit purpose of tissue repair or tissue regeneration. It is necessary to perform the tissue or cell culture in a suitable culture medium with or without stimulus such as stress or orientation. The cells include fibroblasts, chondrocytes, osteoblasts, osteoclasts, osteocytes, synovial cells, bone marrow stromal cells, stem cells, fibrocartilage cells, endothelial cells and smooth muscle cells.

Surfaces on the biodurable reticulated elastomeric matrix possessing different pore morphology, size, shape and orientation may be cultured with different type of cells to develop cellular tissue engineering implantable devices that are specifically targeted towards orthopedic applications, especially in soft tissue attachment, repair, regeneration, augmentation and/or support encompassing the spine, shoulder, knee, hand or joints, and in the growth of a prosthetic organ. In another embodiment, all the surfaces on the biodurable reticulated elastomeric matrix possessing similar pore morphology, size, shape and orientation may be so cultured.

In other embodiments, the biodurable reticulated elastomeric matrix of this invention may have applications in the areas of mammary prostheses, pacemaker housings, LVAD bladders or as a tissue bridging matrix.

### Treatment of Soft Tissue Defects

Implantable device systems incorporating reticulated elastomeric matrix can be used as described in the applications to which priority is claimed. In one embodiment, implantable devices comprising reticulated elastomeric matrix can be used to treat a tissue defect, e.g., for the repair, reconstruction, regeneration, augmentation, gap interposition or any mixture thereof in an orthopedic application, general surgical application, cosmetic surgical application, tissue engineering application, or any mixture thereof.

The exemplary composite surgical mesh for embodiments of the invention is intended for use in general surgical procedures to assist in the repair and/or reinforcement of hernia and other soft tissue defects requiring additional support of a nonabsorbable implant during and after wound healing.

In one embodiment, the implantable device comprising reticulated elastomeric matrix or composite mesh comprising reticulated elastomeric matrix is used for for repair of weakness in biologic connective tissue that allows the bulging or herniation of another organ or organ system(s) with the resultant physiologic impairment. In one embodiment, implantable device comprising reticulated elastomeric matrix or reticulated elastomeric matrix comprising a coating or composite mesh comprising reticulated elastomeric matrix or composite mesh comprising reticulated elastomeric matrix and a coating is used for for repair of hernias and as surgical meshes for augmentation, support and ingrowth. In another embodiment, composite mesh comprising reticulated elastomeric matrix and a coating is used for for repair of hernia and as surgical meshes for augmentation, support and ingrowth. In another embodiment, reticulated elastomeric matrix comprising a coating is used for for repair of hernia and as surgical meshes for augmentation, support and ingrowth. In one embodiment, the coating has anti-adhesive functionality or antiadhesive property or can be used as anti-adhesive barrier.

In one embodiment, the features of the implantable device and its functionality make it suitable for general surgical applications, such as in the repair of a hernia.

The implantable device of the present invention comprising reticulated elastomeric matrix or or reticulated elastomeric matrix comprising a coating or composite mesh comprising reticulated elastomeric matrix or composite mesh comprising reticulated elastomeric matrix and a coating may be use to repair soft tissue defects, such as for example hernia, specifically inguinal, femoral, ventral, incisional, umbilical, and epigastric hernias. In certain embodiments, the device maybe is used in the repair and/or reinforcement of hernia and other soft tissue defects requiring additional support of a nonabsorbable implant during and after wound healing. Preferably, the device is used for the treatment of inguinal or ventral hernias.

Hernias can be generally described as inguinal location or ventral abdominal with other less common but well-know variant locations, i.e., femoral or umbilical. In one embodiment, the hernia to be repaired is an inguinal hernia, a ventral abdominal hernia, a femoral hernia, an umbilical hernia, or any mixture thereof. Hernias located in the anterior or lateral abdominal wall at sites of prior surgery or trauma can be approached directly or via laproscopic approach. The repair essentially places the implantable device comprising reticulated elastomeric matrix within the abdominal wall, thereby augmenting or reinforcing defects in the muscle/facia of the rectus sheath-transversals, external oblique and/or internal oblique. In one embodiment, the implantable device comprising the reticulated elastomeric matrix or or composite mesh comprising reticulated elastomeric matrix can have one side treated to be microporous or smooth on the abdominal cavity-facing side and another porous side for tissue ingrowth into the externally-facing implant. In another embodiment, the implantable device comprising the reticulated elastomeric matrix or or composite mesh comprising reticulated elastomeric matrix can have a coating or surface coating on the abdominal cavity-facing side and another reticulated side for tissue ingrowth into the externally-facing implant. The coating or surface coating can be smooth or somewhat smooth or significantly smooth. In one embodiment, the coating or surface coating has anti-adhesive functionality or antiadhesive property or can be used as anti-adhesive barrier.

The hernia device of the present invention may be use to repair soft tissue defects, such as, for example, specifically inguinal, femoral, ventral, incisional, umbilical, and epigastric hernias. In certain embodiments, the device maybe is used in the repair and/or reinforcement of hernia and other soft tissue defects requiring additional support of a nonabsorbable implant during and after wound healing. Preferably, the device is used for the treatment of inguinal or ventral hernias.

In one embodiment, implantable device comprising reticulated elastomeric matrix or reticulated elastomeric matrix comprising a coating or composite mesh comprising reticulated elastomeric matrix or composite mesh comprising reticulated elastomeric matrix and a coating may be placed to cover a defect (e.g., an inguinal hernia) either directly through a groin incision or using a laparoscope. The device may be placed to cover a defect (e.g., an inguinal hernia) either directly through a groin incision or using a laparoscope. The device may be secured to the affected area by any means. For example, the device may be sutured to the affected area. Alternatively, the device may be used to provide tensionless repair to the affected area. Exemplary methods for tensionless repair of the affected area, include sutureless techniques such as fixation of the device with a glue (e.g., human fibrin glue).

Repair of the hernia by an incision is commonly referred to as “open” hernia repair. Open mesh operations may include, for example, flat mesh, plug and mesh, or peritoneal mesh procedures. See “Repair of Groin Hernia with Synthetic Mesh, Annuals of Surgery, Vol. 235, No. 3, 322-332 (2002). In open hernia repair, the surgeon makes an incision in the groin area and manipulate the hernia back into the abdomen. In one embodiment, inguinal hernia can be approached via a pre-peritoneal approach, i.e., using the internal ring as direct access to the preperitoneal space through an open anterior approach with “tension-free” Lichenstein or plugging or, alternatively, a laproscopic approach.

In Lichtenstein tension-free repair, the inguinal canal is approached from an open anterior approach after dividing the skin, scarpa fascia, and external oblique aponeurosis. The cord is examined for an indirect sac, any direct hernia is reduced, and the floor is reinforced by an implantable device comprising reticulated elastomeric matrix being sewn to the conjoint tendon and the shelving edge of the inguinal ligament. The implantable device comprising reticulated elastomeric matrix can be slit or designed to accommodate the cord structures. In the Kugel technique, a single or bilayer of an implantable device comprising reticulated elastomeric matrix (with or without a self-retaining outer memory recoil ring) is placed anteriorly through a 4 cm muscle-splitting incision in the preperitoneal space.

Alternatively, the device may be placed to cover a hernia by making small incisions in the abdomen for insertion of a laparoscope. Laparoscopic operations may include transabdominal preperitoneal (TAPP) or totally extraperitoneal (TEP) procedures. See id. The surgeon may use the laparoscope in combination with other surgical tools to push back the hernia and secure the device to the affected area. Both the TAPP and TEP can place an implantable device comprising reticulated elastomeric matrix in the preperitoneal space. The TAPP repair is performed from within the abdomen with an incision that is made in the peritoneum to access the preperitoneal space. In the TEP repair, dissection is initiated totally in the extraperitoneal space. Goals of appropriate repair in both approaches include: (1) dissection of the myo-pectineal-orifice (MPO) and surrounding structures completely, with full exposure of the pubic bone medially and the space of Retzius; (2) removal of preperitoneal fat and cord lipomas; (3) assessment of all potential hernia sites; (4) full reduction of direct hernia sac; and (5) skeletonization of the cord to ensure proximal reduction of the indirect sac from the vas deferens and gonadal vessels.

The device may also be used for the treatment of a ventral hernia either by open hernia repair or by laparoscopy, as discussed above. In one embodiment, the device may be placed in the affect area using an extraperitoneal sublay technique, in which the mesh is sutured into place on the posterior rectus sheath with approximately 4 cm of fascia overlap. Peritoneum is closed or omentus is placed between the device and intra-abdominal organs to prevent contact. In a certain embodiment, the device may be placed in the affected area using an inlay technique. In the inlay technique, the device is sutured to the facial edges. Alternatively, the device may be placed in the affected area using an onlay technique whereby the device is placed and sutured onto the anterior rectus sheath. See Penttinen et al., “Mesh repair of common abdominal hernias: a review on experimental and clinical studies,” Hernia 12: 337-344 (2008). In another embodiment, the laparoscopic ventral hernia repair is an intraperitoneal technique in which the device is placed against an intact peritoneum and anchored to the abdominal wall. See Voeller et al., “Advancements in Ventral Hernia Repair, General Surgery News, 35-41 (March 2008). In another embodiment, the laparoscopic ventral hernia repair is an intraperitoneal technique in which the device is placed against an intact peritoneum and anchored to the abdominal wall. See Voeller et al., “Advancements in Ventral Hernia Repair, General Surgery News, 35-41 (March 2008).

Without being bound by any particular theory, it is believed that the implantable device comprising reticulated elastomeric matrix or reticulated elastomeric matrix comprising a coating or composite mesh comprising reticulated elastomeric matrix or composite mesh comprising reticulated elastomeric matrix and a coating provide improved healing, such as shorter healing response time and/or less pain, over time compared to other synthetic meshes. These significant improvements in the devices are believed to arise, from a combination of improved mechanical properties and a much more favorable biologic response in vivo.

From the mechanical property perspective, the implantable devices for repair of hernias such as inguinal and ventral are believed to have better acute handling (i.e. physician handling during placement of the mesh into the defect) for implant procedures than conventional woven, knitted and/or ePTFE films or composites of these materials. Specifically, conventional synthetic meshes while able to conform to a flat surfaces have more difficulty in conforming to complex geometries presented at the anatomic sites of hernias and other soft tissue defects. The difficulty in conforming to complex geometries arise from the from the planar structure of conventional meshes in which knitted or woven meshes must maintain large enough pore sizes to minimize fibrotic encapsulation (or scarring) biologic response but must also have appropriate strength and stiffness to prevent recurrence of the soft tissue defect. At the same time, these devices must have adequate stiffness to allow the physician to easily place the device at the implant site but not too high a stiffness where in the device cannot not be easily placed in the appropriate anatomy. One simple method to measure how a well a material can conform to a shape is to use average device tensile stiffness as a parameter to quantify the handling properties of the whole device such as common surgical meshes such as Mersiline™ or mesh is considered to be one of the most compliant meshes because of it's multifilament construction vs. a monofilament construction of a device such as UltraPro™ which has a much higher stiffness. In fact meshes that do not have enough ‘stiffness’ can be difficult to handle especially for laparoscopic procedures. The devices in this invention Composite Mesh1 (2 layers of reticulated elastomeric matrix reinforced with PP mesh in a “sandwich” configuration) and Composite Meshe 2 (1 layer of reticulated elastomeric matrix reinforced with PP mesh with a coating of 70/30 PLA/PCL copolymer melt-bonded to the PP mesh) have a device tensile stiffness equivalent or slightly lower than Mersiline and and significantly lower than Ultrapro thus achieving a balance between being too stiff and too “floppy” as shown in Table 1. The properties were tested along the machine direction or the stronger direction of theses non-isotropic meshes.

TABLE 1
Comparison of Tensile stiffness of various hernia meshes.
(Gauge lengths and widths of all the samples were the same)

| | Average Stiffness |
|---|---|
| Device | (N/mm) |
| Mersilene Mesh - (Machine Direction) | 0.56 ± 0.01 |
| UltraPro Mesh - (Machine Direction) | 1.77 ± 0.09 |
| Composite mesh 1-2 layers of reticulated | 0.32 ± 0.05 |
| elastomeric matrix reinforced with PP mesh | |
| in a “sandwich” configuration (Machine | |
| Direction) | |
| Composite mesh 2-1 layer of reticulated | 0.32 ± 0.07 |
| elastomeric matrix reinforced with PP mesh | |
| with a coating of 70/30 PLA/PCL | |
| copolymer melt-bonded to the PP mesh | |
| (Machine Direction) | |

Additionally, for laparoscopic placement of meshes, an important parameter is the ability of the device to unfurl, unravel, or recover to it's original flat sheet configuration when exiting the trochar cannula into the body cavity. The devices in this invention by virtue of it's multi layer composite design and the use of an elastomeric adhesive ‘unfurl’ to it's flat sheet configuration with minimal manipulation unlike flat sheet meshes which require manual intervention with graspers intraoperatively to flatten out the sheets once exiting the trochar cannula.

Another advantage of the design construct of both the inguinal and ventral design is the ability to protect the body from direct exposure from the multifilament or monofilament meshes. In certain situations these meshes can become abrasion points in the body where in tissue can abraded by the filaments of the meshes or filaments of common surgical meshes. The device comprising reticulated elastomeric resilient matrix is considered to be soft compared to common surgical meshes and this softness ensures that contact and frictional stresses between the mesh and the surrounding tissue are minimized as a result of the presence of the biomechanical buffering action by the reticulated elastomeric resilient matrix. Without being bound by any particular theory, the softness of the reticulated elastomeric resilient matrix arises from high void content, the segmented polyurethane chemistry comprising a MDI based hard and a polyol based soft segment, a hard segment that is a mixture of 2,4 and 4,4 MDI leading to a disruption of the more ordered or more organized structure of the hard segment and the significant of total absence of the cell walls of the reticulated structure.

It is believed that the improved flexibility provides for improved conformability to the contours of the body and allow for better apposition as compared to other synthetic meshes. It is believed the three dimensional nature of the reticulated elastomeric matrix with the mesh material provides a three-dimensional scaffold that promotes cellular ingrowth and is believed to provide faster healing as compared to medical textiles. In addition, it is believed that the improved healing will reduce long term pain because it is believed that there will be less of a fibrotic “scaring” and less mesh contracture. In addition the device is believed to have a reduced risk for infection because, it is believed that the three-dimensional nature of the device promotes angiogenesis, specifically for new blood vessels to deliver macrophages that would help fight off a local infection around the device.

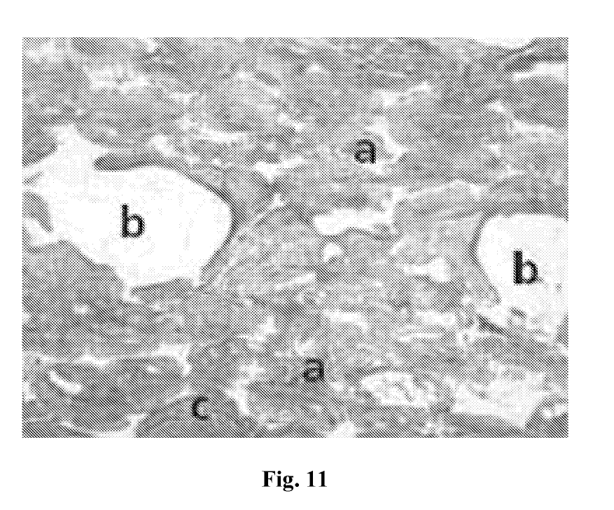

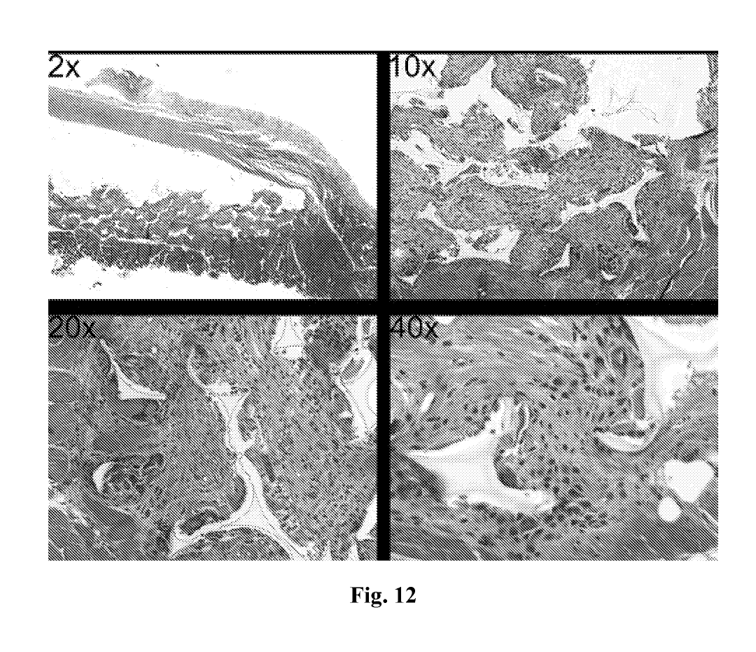

From the perspective of the biologic response elicited by the implanted device, there are a multitude of features that enable more optimal clinical end points and outcomes w.r.t more rapid healing, times, avoidance of the formation of a scar plate (encapsulation), while preventing recurrence of the hernia defects. The open and interconnect pore structure (with 95% accessible void content) of the reticulated elastomeric resilient matrix (and therefore high fluid permeability) combined with the predominantly hydrophobic surface chemistry of the polycarbonate polyurethane urea matrix material allows the implant to rapidly adsorb blood plasma and extracellular matrix proteins from the implantation site within a short time following implantation. The very same permeable morphology of the reticulated structure also allow for the recruitment of natively available cells such as platelets, macrophages, fibroblasts, and locally sources mesenchymal stem cells to adhere and attach to the proteins immobilized on the surface of the reticulated elastomeric matrix. The three-dimensional reticulated structure of the reticulated elastomeric resilient matrix helps in spatial organization of the cells to maximize cell-cell interactions and cell-extracellular matrix interactions. Preclinical studies conducted on animals with implantable device that are composite mesh comprising reticulated elastomeric matrix or composite mesh comprising reticulated elastomeric matrix and a coating indicate that significant collagen deposition occurs very early in the healing process in the presence of the reticulated elastomeric matrix and by 26 weeks, there is a stable wound healing response (FIG. 11)—The matrix material shows very robust and controlled fibroblast infiltration and activity (as evidenced by synthesis and maturation of type 1 collagen within the pores of the device), and early time periods show active angiogenesis. More importantly, there is clear evidence in preclinical studies in the rat model that demonstrate that the device(s) prevent the formation of a fibrous scar capsule and instead allow continuity between adjacent tissue and the tissue deposited within the pores of the biomaterial. The foreign body response is primarily defined by the formation of a thin boundary layer (about 10 microns thick) of macrophages and multi-nucleate giant cells that surround the filaments of the reticulated elastomeric matrix (FIG. 12). The presence of this macrophage response (albeit localized around the microfilaments) and the formation of a robust blood vessel network within the pores of the device, is a direct consequence of the open pore interconnected morphology which ensures that the device allows the body's immune system has access to the interior voids of the matrix, thus ensuring a reduced infection risk. The device is also resistant to degradation through the oxidative and hydrolytic pathways by virtue of the crosslinked chemistry of the reticulated elastomeric matrix. Macrophages are known to produce reactive oxygen species as the primary pathway to degrade and breakdown foreign bodies implanted in vivo. Many studies have shown that reticulated elastomeric matrix are specifically resistant to this type of oxidative degradation. All these features of the device, i.e., open interconnected pores, high fluid permeability, elastomeric resilience, and resistance to oxidative/hydrolytic degradation can therefore be considered the novel and improved functionalities that lead to the favorable biologic wound healing response, while at the same time present a biomechanical suitable device to prevent the recurrence of hernia at the implantation site(s).

In another embodiment, implantable devices comprising reticulated elastomeric matrix can be used in an orthopedic application for the repair, reconstruction, regeneration, augmentation, gap interposition or any mixture thereof of tendons, ligaments, cartilage, meniscus, spinal discs or any mixture thereof. For example, implantable devices comprising reticulated elastomeric matrix can be used in a wide range of orthopedic applications, including but not limited to repair and regeneration encompassing the spine, shoulder, elbow, wrist, hand, knee, ankle, or other joints, as discussed in detail in priority applications. attachment, regeneration, augmentation or support of soft tissues including ligament In one non-limiting example, the compression set, resilience and/or recovery of the implantable device is engineered to provide high recovery force of the reticulated elastomeric matrix after repetitive cyclic loading. Such a feature is particularly advantageous in orthopedic and for hernia uses in which cylic loading of the implantable device might otherwise permanently compress the reticulated elastomeric matrix, thereby preventing it from achieving the substantially continuous contact with the surrounding soft tissues necessary to permit optimal cellular infiltration and tissue ingrowth. In another non-limiting example, the density and pore size of an implantable device is engineered to provide acceptable permeability of the reticulated elastomeric matrix under compression. Such features are advantageous in spine and knee orthopedic applications, in which high loads are placed on the implantable device. In yet another non-limited example, the properties of the reticulated elastomeric matrix are engineered to maximize its “soft, conformal fit,” particularly advantageous in cosmetic surgical applications. In a further, non-limiting example, the tensile properties of the implantable device are maximized to complement the fixation technique used, e.g., to provide maximum resistance to suture pullout.

In a further embodiment, the implantable devices disclosed herein can be used as a drug delivery vehicle. For example, a therapeutic agent can be mixed with, covalently bonded to, adsorbed onto and/or absorbed into the biodurable solid phase 12. Any of a variety of therapeutic agents can be delivered by the implantable device, for example, those therapeutic agents previously disclosed herein.

The device is believed to provide improved healing, such as shorter healing response time and/or less pain, over time compared to other synthetic meshes. These improvements are believed to arise, at least in part, from the mechanical properties of the device. Specifically, the device is more flexible then devices formed from medical textiles. It is believed that the improved flexibility provides for improved conformability to the contours of the body and allow for better apposition as compared to other synthetic meshes. It is believed the three dimensional nature of the reticulated elastomeric matrix with the mesh material provides a three-dimensional scaffold that promotes cellular ingrowth and is believed to provide faster healing as compared to medical textiles. In addition, it is believed that the improved healing will reduce long term pain because it is believed that there will be less of a fibrotic “scaring” and less mesh contracture. In addition the device is believed to have a reduced risk for infection because, it is believed that the three-dimensional nature of the device promotes angiogenesis, specifically for new blood vessels to deliver macrophages that would help fight off a local infection around the device.

### Examples

### Example 1

### Synthesis and Properties of Reticulated Elastomeric Matrix for Embodiments of the Invention (Hereinafter “Reticulated Elastomeric Matrix 1”)

A reticulated cross-linked biodurable elastomeric polycarbonate urea-urethane matrix was made by the following procedure.

The aromatic isocyanate MONDUR MRS-20 (from Bayer Corporation) was used as the isocyanate component. MONDUR MRS-20 is a liquid at 25° C. MONDUR MRS-20 contains 4,4′-diphenylmethane diisocyanate (MDI) and 2,4′-MDI and has an isocyanate functionality of about 2.2 to 2.3. A diol, poly(1,6-hexanecarbonate) diol (POLY-CD220 from Arch Chemicals) with a molecular weight of about 2,000 Daltons, was used as the polyol component and was a solid at 25° C. Distilled water was used as the blowing agent. The catalysts used were the amines triethylene diamine (33% by weight in dipropylene glycol; DABCO 33LV from Air Products) and bis(2-dimethylaminoethyl)ether (23% by weight in dipropylene glycol; NIAX A-133 from GE Silicones). Silicone-based surfactants TEGOSTAB BF 2370 and TEGOSTAB B-8305 (from Goldschmidt) were used for cell stabilization. A cell-opener was used (ORTEGOL 501 from Goldschmidt). The viscosity modifier propylene carbonate (from Sigma-Aldrich) was present to reduce the viscosity. Glycerine (99.7% USP Grade) and 1,4-butanediol (99.75% by weight purity, from Lyondell) were added to the mixture as, respectively, a cross-linking agent and a chain extender. The proportions of the ingredients that were used is given in Table 2 below.

TABLE 2

| Ingredient | Parts by Weight |
|---|---|
| Polyol Component | 100 |
| Isocyanate Component | 52.96 |
| Isocyanate Index | 1.00 |
| Viscosity Modifier | 5.80 |
| Cell Opener | 2.00 |
| Distilled Water | 1.95 |
| B-8305 Surfactant | 0.70 |
| BF 2370 Surfactant | 0.70 |
| 33LV Catalyst | 0.45 |
| A-133 Catalyst | 0.12 |
| Glycerine | 2.00 |
| 1,4-Butanediol | 0.80 |

The isocyanate index, a quantity well known in the art, is the mole ratio of the number of isocyanate groups in a formulation available for reaction to the number of groups in the formulation that are able to react with those isocyanate groups, e.g., the reactive groups of diol(s), polyol component(s), chain extender(s), water and the like, when present. The isocyanate component of the formulation was placed into the component A metering system of an Edge Sweets Bench Top model urethane mixing apparatus and maintained at a temperature of about 20-25° C.

The polyol was liquefied at about 70° C. in an oven and combined with the viscosity modifier and cell opener in the aforementioned proportions to make a homogeneous mixture. This mixture was placed into the component B metering system of the Edge Sweets apparatus. This polyol component was maintained in the component B system at a temperature of about 65-70° C.

The remaining ingredients from Table 2 were mixed in the aforementioned proportions into a single homogeneous batch and placed into the component C metering system of the Edge Sweets apparatus. This component was maintained at a temperature of about 20-25° C. During foam formation, the ratio of the flow rates, in grams per minute, from the supplies for component A:component B:component C was about 8:16:1.

The above components were combined in a continuous manner in the 250 cc mixing chamber of the Edge Sweets apparatus that was fitted with a 10 mm diameter nozzle placed below the mixing chamber. Mixing was promoted by a high-shear pin-style mixer operating in the mixing chamber. The mixed components exited the nozzle into a rectangular cross-section release-paper coated mold. Thereafter, the foam rose to substantially fill the mold. The resulting mixture began creaming about 10 seconds after contacting the mold and was at full rise within 120 seconds. The top of the resulting foam was trimmed off and the foam was placed into a 100° C. curing oven for 5 hours.

Following curing, the sides and bottom of the foam block were trimmed off and the foam was placed into a reticulator device comprising a pressure chamber, the interior of which was isolated from the surrounding atmosphere. The pressure in the chamber was reduced so as to remove substantially all the air in the cured foam. A mixture of hydrogen and oxygen gas, present at a ratio sufficient to support combustion, was charged into the chamber. The pressure in the chamber was maintained above atmospheric pressure for a sufficient time to ensure gas penetration into the foam. The gas in the chamber was then ignited by a spark plug and the ignition exploded the gas mixture within the foam. To minimize contact with any combustion products and to cool the foam, the resulting combustion gases were removed from the chamber and replaced with about 25° C. nitrogen immediately after the explosion. Then, the above-described reticulation process was repeated. Without being bound by any particular theory, the explosions were believed to have at least partially removed many of the cell walls or “windows” between adjoining cells in the foam, thereby creating open pores and leading to a reticulated elastomeric matrix structure.

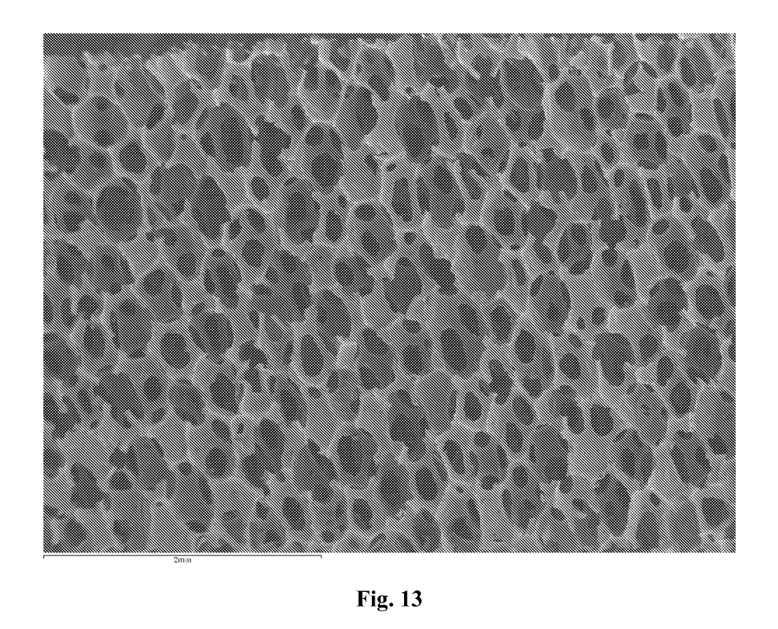

The average cell diameter or other largest transverse dimension of Reticulated Elastomeric Matrix 1, as determined from optical microscopy observations, was about 525 μm. FIG. 13 is a scanning electron micrograph (SEM) image of Reticulated Elastomeric Matrix 1 demonstrating, e.g., the network of cells interconnected via the open pores therein and the communication and interconnectivity thereof. The scale bar at the bottom edge of FIG. 13 corresponds to about 500 μm. The average pore diameter or other largest transverse dimension of Reticulated Elastomeric Matrix 1, as determined from SEM observations, was about 205 μm.

The following tests were carried out on the thus-formed Reticulated Elastomeric Matrix 1, obtained from reticulating the foam, using test methods based on ASTM Standard D3574. Bulk density was measured using Reticulated Elastomeric Matrix 1 specimens of dimensions 5.0 cm×5.0 cm×2.5 cm. The post-reticulation density was calculated by dividing the weight of the specimen by the volume of the specimen. A density value of 3.29 lbs/ft³ (0.053 g/cc) was obtained.

Tensile tests were conducted on Reticulated Elastomeric Matrix 1 specimens that were cut either parallel to or perpendicular to the foam-rise direction. The dog-bone shaped tensile specimens were cut from blocks of reticulated elastomeric matrix. Each test specimen measured about 0.5 cm thick, about 1.25 cm wide, and about 18 cm gauge length. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/min). The average post-reticulation tensile strength perpendicular to the foam-rise direction was determined to be about 34.3 psi (24,115 kg/m²). The post-reticulation elongation to break perpendicular to the foam-rise direction was determined to be about 124%. The average post-reticulation tensile strength parallel to the foam-rise direction was determined to be about 61.4 psi (43,170 kg/m²). The post-reticulation elongation to break parallel to the foam-rise direction was determined to be about 122%.

Compressive tests were conducted using Reticulated Elastomeric Matrix 1 specimens measuring 5.0 cm×5.0 cm×2.5 cm. The tests were conducted using an INSTRON Universal Testing Instrument Model 1122 with a cross-head speed of 1 cm/min (0.4 inches/min). The post-reticulation compressive strength at 50% compression, parallel to the foam-rise direction, was determined to be about 2.1 psi (1,475 kg/m²). The post-reticulation compression set, determined after subjecting the reticulated specimen to 50% compression for 22 hours at 25° C. then releasing the compressive stress, parallel to the foam-rise direction, was determined to be about 8.5%.

The static recovery of Reticulated Elastomeric Matrix 1 was measured by subjecting cylindricular specimens, each 12 mm in diameter and 6 mm in thickness, to a 50% uniaxial compression in the foam-rise direction using the standard compressive fixture in a Q800 Dynamic Mechanical Analyzer (TA Instruments, New Castle, Del.) for 120 minutes followed by 120 minutes of recovery time. The time required for recovery to 90% of the specimen's initial thickness of 6 mm (“t-90%”) was measured and the average determined to be 1406 seconds.

The resilient recovery of Reticulated Elastomeric Matrix 1 was measured by subjecting rectangular parallelepiped specimens, each 1 inch (2.54 cm) high (in the foam-rise direction)×1.25 inches×1.25 inches (3.18 cm×3.18 cm), to a 50% uniaxial compression in the foam-rise direction and then, while maintaining that uniaxial compression, imparting, in an air atmosphere, a dynamic loading of ±5% strain at a frequency of 1 Hz for 5,000 cycles or 100,000 cycles, also in the foam-rise direction. Additionally, rectangular parallelepiped specimens were also tested as described above for 100,000 cycles except that the samples were submerged in water throughout the testing. The time required for recovery to 67% (“t-67%”) and 90% (“t-90%”) of the specimens' initial height of 1 inch (2.54 cm) was measured and recorded. The results obtained are shown in Table 3 below.

TABLE 3

| | Test Specimen | | |
|---|---|---|---|
| No. of Cycles at | Orientation | | |
| 50% Compression ± | Relative to Foam- | t-67% | t-90% |
| 5% Strain at 1 Hz | Rise Direction | (sec) | (sec) |
| 5,000 (in air) | Parallel | 0.7 | 46 |
| 100,000 (in air) | Parallel | 84 | 2370 |
| 100,000 (in water) | Parallel | — | 3400 |

Fluid, e.g., liquid, permeability through Reticulated Elastomeric Matrix 1 was measured in the foam-rise direction using an Automated Liquid Permeameter—Model LP-101-A (also from Porous Materials, Inc.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeameter apparatus. To measure liquid permeability, water was allowed to extrude upward, driven by pressure from a fluid reservoir, from the specimen's end through the specimen along its axis. The operations associated with permeability measurements were fully automated and controlled by a Capwin Automated Liquid Permeameter (version 6.71.92) which, together with Microsoft Excel software, performed all the permeability calculations. The permeability of Reticulated Elastomeric Matrix 1 was determined to be 498 Darcy in the foam-rise direction.

Permeability was also measured after Reticulated Elastomeric Matrix 1 was compressed (perpendicular to the foam-rise direction) so as to reduce the available flow area, thereby simulating compressive molded samples. This was done by inserting a cylindrical sample, with a diameter greater than the diameter of the stainless steel sample holder, into the holder, thereby radially compressing the sample. The uncompressed cylindrical Reticulated Elastomeric Matrix 1 specimens tested were about 7.0 mm in diameter and 13-14 mm in length, while the diameter of the compressed samples ranged from about 9.0 mm to about 16.0 mm prior to their compression into the about 7.0 mm diameter stainless steel holder. For example, the permeability in the foam-rise direction for Reticulated Elastomeric Matrix 1 decreased to 329 Darcy when the available flow area after compression was reduced to 47.9% of the original area and to 28 Darcy when the available flow area after compression was reduced to 19.4% of the original area.

### Example 2

### Synthesis and Properties of Reticulated Elastomeric Matrix for Other Embodiments of the Invention (Hereinafter “Reticulated Elastomeric Matrix 2”)

A reticulated cross-linked biodurable elastomeric polycarbonate urea-urethane matrix was made by the following procedure.

The aromatic isocyanate MONDUR 1488 (from Bayer Corporation) was used as the isocyanate component. MONDUR 1488 is a liquid at 25° C. MONDUR 1488 contains 4,4′-diphenylmethane diisocyanate (MDI) and 2,4′-MDI and has an isocyanate functionality of about 2.2 to 2.3. A diol, poly(1,6-hexanecarbonate) diol (POLY-CD220 from Arch Chemicals) with a molecular weight of about 2,000 Daltons, was used as the polyol component and was a solid at 25° C. Distilled water was used as the blowing agent. The catalysts used were the amines triethylene diamine (33% by weight in dipropylene glycol; DABCO 33LV from Air Products) and bis(2-dimethylaminoethyl)ether (23% by weight in dipropylene glycol; NIAX A-133 from Momentive). Silicone-based surfactants TEGOSTAB BF2370, B8300, and B5055 (from Evonik Degussa) were used for cell stabilization. A cell-opener was used (ORTEGOL 501 from Evonik Degussa). Glycerine (99.7% USP Grade) and 1,4-butanediol (99.75% by weight purity, from Lyondell) were added to the mixture as, respectively, a cross-linking agent and a chain extender. The proportions of the ingredients that were used is given in Table 4 below.

TABLE 4

| Ingredient | Parts by Weight |
|---|---|
| Polyol Component | 100 |
| Isocyanate Component | 45.58 |
| Cell Opener | 3.00 |
| Distilled Water | 1.60 |
| BF2370 Surfactant | 1.20 |
| B8300 Surfactant | 0.60 |
| B5055 Surfactant | 0.60 |
| 33LV Catalyst | 0.40 |
| A-133 Catalyst | 0.15 |
| Glycerine | 1.00 |
| 1,4-Butanediol | 1.50 |

The isocyanate component of the formulation was placed into the component A metering system of the urethane production equipment and maintained at a temperature of about 20-25° C. The isocyanate index, a quantity well known in the art, is the mole ratio of the number of isocyanate groups in a formulation available for reaction to the number of groups in the formulation that are able to react with those isocyanate groups, e.g., the reactive groups of diol(s), polyol component(s), chain extender(s), water and the like, when present. An isocyanate index of 1.0 was used.

The polyol component was liquefied at about 70° C. in an oven. This polyol component was placed into the component B metering system of the urethane production equipment. This polyol component was maintained in the component B system at a temperature of about 65-70° C.

The cell opener component of the formulation was placed into the component C metering system of the urethane production equipment and maintained at a temperature of about 20-25° C.

The remaining ingredients from Table 4 were mixed in the aforementioned proportions into a single homogeneous batch and placed into the component D metering system of the urethane production equipment. This component was maintained at a temperature of about 20-25° C. During foam formation, the ratio of the flow rates, in grams per minute, from the supplies for component A:component B:component C:component D was about 15:33:2:1.

The above components were combined in a continuous manner in the 70 cc mixing chamber of the Max Urethane mixhead of the urethane production equipment. Mixing was promoted by a high-shear pin-style mixer operating in the mixing chamber at a rotational speed of 7000 rpm. The mixed components exited the nozzle onto a release paper coated conveyor belt continuous mold. Thereafter, the foam rose to substantially fill the mold. The resulting mixture began creaming about 10 seconds after contacting the mold and was at full rise within 120 seconds. The top of the resulting foam was trimmed off and the foam was placed into a 100° C. curing oven for 5 hours.

Following curing, the sides and bottom of the foam block were trimmed off then the foam was placed into the reticulator process equipment comprising a pressure chamber, the interior of which was isolated from the surrounding atmosphere. The pressure in the chamber was reduced so as to remove substantially all the air in the cured foam. A mixture of hydrogen and oxygen gas, present at a ratio sufficient to support combustion, was charged into the chamber. The pressure in the chamber was maintained above atmospheric pressure for a sufficient time to ensure gas penetration into the foam. The gas in the chamber was then ignited by a spark plug and the ignition exploded the gas mixture within the foam. To minimize contact with any combustion products and to cool the foam, the resulting combustion gases were removed from the chamber and replaced with about 25° C. nitrogen immediately after the explosion. Then, the above-described reticulation process was repeated one more time. Without being bound by any particular theory, the explosions were believed to have at least partially removed many of the cell walls or “windows” between adjoining cells in the foam, thereby creating open pores and leading to a reticulated elastomeric matrix structure.

The typical average cell diameter or other largest transverse dimension of Reticulated Elastomeric Matrix 2, as determined from optical microscopy observations, was about 336 μm. FIG. 13 is a scanning electron micrograph (SEM) image of Reticulated Elastomeric Matrix 2 demonstrating, e.g., the network of cells interconnected via the open pores therein and the communication and interconnectivity thereof. The scale bar at the bottom edge of FIG. 13 corresponds to about 2000 μm. The average pore diameter or other largest transverse dimension of Reticulated Elastomeric Matrix 2, as determined from SEM observations, was about 250 μm.

The following tests were carried out on the thus-formed Reticulated Elastomeric Matrix 2, obtained from reticulating the foam, using test methods based on ASTM Standard D3574. Bulk density was measured using Reticulated Elastomeric Matrix 2 specimens of dimensions 5.0 cm×5.0 cm×2.5 cm. The post-reticulation density was calculated by dividing the weight of the specimen by the volume of the specimen. A typical density value of 3.62 lbs/ft³ (0.058 g/cc) was obtained.

Tensile tests were conducted on Reticulated Elastomeric Matrix 2 specimens that were cut either parallel to or perpendicular to the foam-rise direction. The dog-bone shaped tensile specimens were cut from blocks of reticulated elastomeric matrix. Each test specimen measured about 1.25 cm thick, about 2.54 cm wide, and about 14 cm long. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/min). The typical average post-reticulation tensile strength perpendicular to the foam-rise direction was determined to be about 50.81 psi (35,567 kg/m²). The typical post-reticulation elongation to break perpendicular to the foam-rise direction was determined to be about 279%. The typical average post-reticulation tensile strength parallel to the foam-rise direction was determined to be about 86.6 psi (60,625 kg/m²). The typical post-reticulation elongation to break parallel to the foam-rise direction was determined to be about 228%.

Compressive tests were conducted using Reticulated Elastomeric Matrix 2 specimens measuring 5.0 cm×5.0 cm×2.5 cm. The tests were conducted using an INSTRON Universal Testing Instrument Model 1122 with a cross-head speed of 1 cm/min (0.4 inches/min). The typical post-reticulation compressive strength at 50% compression, parallel to the foam-rise direction, was determined to be about 1.49 psi (1,040 kg/m²).

The static recovery of Reticulated Elastomeric Matrix 2 was measured by subjecting cylindricular specimens, each 12 mm in diameter and 6 mm in thickness, to a 50% uniaxial compression in the foam-rise direction using the standard compressive fixture in a Q800 Dynamic Mechanical Analyzer (TA Instruments, New Castle, Del.) for 120 minutes followed by 120 minutes of recovery time. The typical time required for recovery to 90% of the specimen's initial thickness of 6 mm (“t-90%”) was measured and the average determined to be 30 seconds.

Fluid, e.g., liquid, permeability through Reticulated Elastomeric Matrix 2 was measured in the foam-rise direction using an Automated Liquid Permeameter—Model LP-101-A (also from Porous Materials, Inc.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeameter apparatus. To measure liquid permeability, water was allowed to extrude upward, driven by pressure from a fluid reservoir, from the specimen's end through the specimen along its axis. The operations associated with permeability measurements were fully automated and controlled by a Capwin Automated Liquid Permeameter (version 6.71.92) which, together with Microsoft Excel software, performed all the permeability calculations. The typical permeability of Reticulated Elastomeric Matrix 2 was determined to be 443 Darcy in the foam-rise direction.

### Example 3

### Fabrication of Composite Made from Reticulated Elastomeric Matrix Reinforced with 2-Dimensional Mesh Reinforcement

The process for manufacturing implantable composite device for embodiments of the invention is described next. Reticulated Elastomeric Matrix 2 was made following the procedures described in the foregoing Example 2. Implantable devices, shaped as rectangular sheets having approximate dimensions of 150 mm in length, 120 mm in width and 0.9 mm in thickness, were cut by machining from Reticulated Elastomeric Matrix 2. Two sheets or substrates were machined.

A knitted polypropylene monofilament fibers (diameters approximately 0.10 mm) in a mesh configuration having a thickness of approximately 0.41 mm, largest grid size ˜1.4 mm×1.2 mm and a Mesh Areal Density of 46-54 g/m² was used as the 2 dimensional mesh reinforcement. The PP mesh was sized similar to the machined Reticulated Elastomeric Matrix 2.

A Silicone adhesive (Nusil™ MED2-4213) was used to bond the PP mesh to the two sheets or substrates of Reticulated Elastomeric Matrix 2.

The manufacturing sequence began with preparation of the polypropylene mesh layer utilizing a surface treatment system consisting of a 3DT Polydyne3 Corona Discharge Generator with controlled translation rate and electrode gap with two passes of the electrode over the mesh. A coating fixture consisting of a movable and height adjustable blade was used to uniformly spread silicone adhesive on to a base plate. A three step silicone adhesive coating process, (involving laying down a layer of silicone on the base plate and transferring the thin layer of silicone on to the PP mesh) was performed that insured uniform application of adhesive to both sides of the PP mesh filaments while maintaining a fully open grid structure of the PP mesh. Then the adhesive coated mesh was sandwiched between two sheets or substrates of machined Reticulated Elastomeric Matrix 2 (washed by using tumbling and sonication by IPA) sheets utilizing tooling that applied compression (using perforated steel plates) to the laminate during heat curing. Shims were used to control the thickness of the sandwiched layer. Silicone was cured at 100° C. for approximately 60 minutes. The tooling was cooled and the silicone bonded sandwiched composite from Reticulated Elastomeric Matrix 2 reinforced with 2 dimensional mesh reinforcement was obtained. The silicone bonded sandwiched composite were washed using sonicating baths containing isopropyl alcohol followed by tumbling in IPA.

The thickness of the composite was approximately 2 mm. The average coat weight of the silicone adhesive was measured to be about 17 mg milligram/cm² of the surface of the elastomeric matrix.

Each implantable composite device, incorporating the PP mesh, was tested for suture retention strength (SRS), which is defined as the maximum force required to pull a standard suture through the device, thereby causing it to fail. Each composite device, incorporating the PP mesh, was also tested for the tensile break strength (TBS), which is defined as the maximum force required for tensile failure for the entire device. Each composite device, incorporating the PP mesh, was also tested for burst strength (BS). All three tests were carried out using a using an INSTRON Universal Testing Instrument Model 3342.

In SRS testing, a 2 0 ETHIBOND braided polyester suture was inserted into one end of the implantable device by using a needle and the suture was attached to the device from 2 mm to 3 mm below the first horizontal grid line and about at the device's center line. A loop, about 50 mm to 60 mm in length, was formed by the two ends of the suture strands. The free end (that was not attached to the suture) of the device was mounted within the flat rubber-coated faces of the bottom fixed jaw and clamped. A schematic of the test is shown in FIG. 10. The SRS test was run under displacement mode at a cross-head speed of 100 mm/min (3.94 in/min) with the movable jaws separating or moving upwards and away from the fixed jaws. An average SRS value of 27±4 Newtons was obtained from testing these implantable composite devices incorporating the PP mesh.

In the TBS testing of these implantable composite devices, one end of the device was mounted between the rubber-coated faces mounted onto the fixed pneumatic grip and the other end of the device was mounted between the rubber-coated faces mounted on the movable pneumatic grip. The test was run under displacement mode at a cross-head speed of 100 mm/min (3.94 in/min) with the movable jaws separating or moving upwards and away from the fixed jaws. An average TBS value of 216±25 Newtons was obtained.

In BS testing of these implantable composite devices, a 25.4 mm ball (held in a movable frame) was pushed through a circular patch of the device held in a retaining ring adapter fixed to a stationary frame. The ball burst test was run at a rate of 4 in/min (102 mm/min) with the movable frame moving downwardly. The test was run until the specimen ruptured which indicated completion of the test. The load-displacement graph was monitored to yield the maximum load (or the ball burst strength). An average BS value of 352±51 Newtons was obtained.

Permeability of the implantable composite devices were approximately equivalent to that of the substrate of the Reticulated Elastomeric Matrix 2.

### Example 4

### Use of Composite of Reticulated Elastomeric Matrix 2 with 2-Dimensional Mesh Reinforcement Implanted in an Abdominal Wall of a Rat

An implantable device formed from Reticulated Elastomeric Matrix 2 and reinforced with the 2-dimensional mesh reinforcement made as described in Example 3 was used to determine the histomorphologic tissue response of the test article in a rat body wall repair model. Twenty-four rats (Sprague-Dawley, male 300-500 g) were used in this study. Each rat was subjected to the removal of a 1 cm by 1 cm portion of the ventral lateral abdominal wall and subsequent replacement of the experimentally-induced body wall defect with the test article.

Aseptic procedures were followed for all procedures. The site was prepared for sterile surgery by clipping of the fur, and scrubbed with sterile saline, betadine solution, and sterile 4×4 gauze. The animal was draped with a small sterile cover leaving the abdominal surgical site exposed. A midline ventral and lateral abdominal incision was made and a partial thickness resection of the abdominal wall was done, leaving the peritoneum and transversals fascia on the interior portion of the wall and the skin on the exterior portion intact. Stated differently, the internal and external abdominal oblique muscles were excised and repaired using the test article. The 1 cm×1 cm defects were filled using the approximately 1 cm×1 cm composite mesh test article which was sutured to the adjacent abdominal wall tissue with prolene non-resorbable suture material. The skin was closed in standard surgical fashion using resorbable suture (vicryl). The animals then recovered from anesthesia and were allowed normal ambulation and diet for the remainder of the study.

The test group was subdivided into six subgroups (n=4 animals/subgroup) based upon time to sacrifice: 1, 2, 4, 8, 16 and 26 weeks. At the specified time point the animals were euthanized and the implant site harvested for histological evaluation. The implant site along with adjacent native tissue were removed and fixed in 10% neutral buffered formalin (NBF). At the time of sacrifice the operative site plus surrounding native tissue was explanted and prepared for histologic methods. Hematoxylin & eosin (H&E) and Masson's trichrome were used in the histologic examination. Microscopic evaluations included the semiquantitative determination of the presence of the test article, angiogenesis, cellular infiltration, multinucleate giant cells, a fibrous connective tissue layer surrounding the device, and host neo-ECM deposition. In addition, measurements (length and width) were taken of devices implanted for 26 weeks.

Gross examination of the 24 explanted devices consistently showed a smooth connective tissue facial covering that adhered to the overlying skin. The implanted devices did not show signs of degradation and there was no evidence of adjacent tissue necrosis.

The host response to the test article or the device consisted of a dense mononuclear cell infiltration beginning in Week 1 accompanied by the formation of increasingly organized connective tissue within and surrounding the graft. The amount of vasculature within the implant increased during the early stages of tissue remodeling and then moderated. The number of multinucleate giant cells increased from week 1 to week 2 and then stabilized; most were seen adjacent to implanted device material. The test article material was present at all time points evaluated and there was no necrosis of the host tissue surrounding the implanted devices at any time point. A well-defined connective tissue layer that integrated with the dense connective tissue stroma was present. Multinucleate giant cells were present near the device material. FIG. 11 is a histology analysis photograph of Rat Body Wall Repair at 26 Weeks (Trichrome Stain 4×) showing (a) Biomerix Biomaterial—PCPU scaffold, (b) 2 dimensional Polypropylene reinforcing Mesh, and (c) Muscle Fibers. In summary, it was observed by 26 weeks (as shown in FIG. 11) that the test article or the device showed a well-tolerated, long term histomorphologic response in the rat abdominal wall model, with good integration with surrounding tissue, minimal foreign body response, and no evidence of device degradation or adjacent tissue necrosis. There was very moderate shrinkage at 26 weeks of an average of 15%.

### Example 5

### Fabrication of Composite from One Layer of Reticulated Elastomeric Matrix Reinforced with 2-Dimensional Mesh Reinforcement and a Film of Biocompatible Polymer that Act as Anti-Adhesive Coating

The process for manufacturing an implantable composite device with anti-adhesive coating for embodiments of the invention is described next. Reticulated Elastomeric Matrix 2 was made following the procedures described in Example 2. Implantable devices, shaped as rectangular sheets having approximately dimensions of 150 mm in length, 120 mm in width and 0.9 mm in thickness, were cut by machining from Reticulated Elastomeric Matrix 2. One sheet or substrate was machined.

A knitted polypropylene monofilament fibers (diameters approximately 0.10 mm) in a mesh configuration having a thickness of approximately 0.41 mm and a Mesh Areal Density of 46-54 g/m² was used as the two dimensional mesh reinforcement. The PP mesh, is sized similar to the machined Reticulated Elastomeric Matrix.

A Silicone adhesive (Nusil™ MED2-4213) was used to bond the PP mesh to the single sheet or substrate of Reticulated Elastomeric Matrix 2.

The anti-adhesion coating materials was (a copolymer of poly (L-lactide co ε-caprolactone) in the molar ratio 70:30) and also known as cap/lac 30/70 provided a flexible coating designed to minimize adhesions while biodegrading within a year. A film was made from the copolymer using a single screen extruder with a maximum barrel temperature of 165° C. and a die with a 4 inch width. The thickness of the cap/lac 30/70 was approximately 110 microns. The inherent viscosity of the cap/lac pellets was between 1.2 and 1.8 dl/g and its melting point is about 112° C.

The cap/lac 30/70 film sheet is then re-melted and bonded to the PP mesh (previously treated with corona discharge in the same way described in Example 3) using precision-ground stainless steel tooling to apply uniform compressive loads to both surfaces. An inert convection oven (using nitrogen) is then used to provide sufficient heat (140 C for 20 minutes) to allow the cap/lac film to flow approximately 0.2 mm into the mesh grid without migrating to the other side. The final coated composite is created by bonding the cap/lac film-PP mesh construct to the reticulated elastomeric matrix using silicone adhesive. This is achieved by embedding the mesh side of the film/mesh construct into a thin film (0.254 mm) of silicone adhesive in order to transfer sufficient adhesive to the mesh necessary to engage the reticulated elastomeric matrix sheet and maintain a fully open structure at the interface. Fixed-gap tooling similar to that used for the film/mesh construct is used in conjunction with convective heat (100 C for 30 minutes) to cure the silicone. The implantable composite devices with anti-adhesive coating are the trimmed to final size and washed in sonicating baths containing isopropyl alcohol.

The thickness of the composite was approximately 1 mm. The average coat weight of the silicone adhesive was measured to be about 7 mg milligram/cm² of the surface of the elastomeric matrix.

Each implantable composite devices with anti-adhesive coating, incorporating the PP mesh and the cap/lac film was tested for suture retention strength (SRS), tensile break strength (TBS), and burst strength (BS) using the test methods described in foregoing Example 3. An average SRS value of 35±6 Newtons was obtained from testing these implantable composite devices with anti-adhesive coating. An average TBS value of 212±25 Newtons was obtained from testing these implantable composite devices with anti-adhesive coating. An average BS value of 326±51 Newtons was obtained from testing these implantable composite devices with anti-adhesive coating

Permeability of the implantable composite devices after removal of the cap/lac layer by contacting with chloroform was approximately equivalent to that of the substrate of the Reticulated Elastomeric Matrix 2.

### Example 6

### Use of Composite of Reticulated Elastomeric Matrix 2 with 2-Dimensional Mesh Reinforcement Implanted and a Film of Biocompatible Polymer that Act as Anti-Adhesive Coating in an Rat Partial Abdominal Wall Defect Model

An implantable device formed from one layer of Reticulated Elastomeric Matrix 2 for embodiments of the invention reinforced with the 2-dimensional mesh reinforcement and a film of biocompatible polymer that act as an anti-adhesive coating was made as described in foregoing Example 5. The host response to the implant or device was compared to a commercially available coated polypropylene ventral hernia repair device (PROCEED™, Ethicon Inc.) in a rat partial abdominal wall defect model.

There were two experimental groups determined by the type of device used for the repair of the rat partial abdominal wall defect: a) Reticulated Elastomeric Matrix 2 reinforced with polypropylene mesh and coated with a Poly L-Lactide-co ε-Caprolactone film layer (coated reinforced composite mesh) and b) PROCEED™ polypropylene mesh (PROLENE Soft Mesh) with oxidized regenerated cellulose (ORC).

Thirty-two rats (Sprague-Dawley, male 300-500 g) were randomly divided into eight groups of four animals each, based upon survival time (1, 2, 4, or 8 weeks). Each rat was subjected to surgical excision of a 1.0 cm2 section of the musculotendinous portion of the ventral lateral abdominal wall, with the abdominal fascia, transversus abdominis, and peritoneum being left intact. The defect was repaired with either the (coated reinforced composite mesh) device (4 groups=16 animals) or the PROCEED™ device for hernia repair (4 groups=16 animals).

Each animal was anesthetized with isoflurane (2% in oxygen) in an inhalation chamber. The surgical site was clipped, shaved, and prepared for sterile surgery with a Betadine (providone-iodine) scrub. Sterile technique was used at all times. A ventral midline abdominal incision was made, and the skin and subcutaneous tissue were separated from the underlying muscle tissues on one side of the midline for a distance of approximately 4.0 cm. The incision in the ventral midline of the abdominal skin was retracted to expose the ventral lateral wall adjacent to the linea alba, including the musculotendinous junction of the abdominal wall musculature. A 1.0 cm2 defect of the musculotendinous portion of the ventral lateral abdominal wall was excised, with the underlying transversals fascia and peritoneum being left intact. Uniformity of the defect size and shape was ensured by using a device with a fixed size and shape on each animal. The defect was then replaced with a 1.0 cm2 piece of the test article chosen for that animal. One 4-0 Prolene suture was placed at each of the four corners of the test article to secure attachment to the adjacent abdominal wall and to demarcate the implant. Securing the test articles in this manner provided a mechanism by which the test article was subjected to the mechanical forces delivered by the adjacent native abdominal wall musculature, while avoiding the predominance of a host tissue reaction to the suture material rather than the test article. A subcuticular placement of 4-0 Vicryl was used to close the skin incision.

Each animal was left to recover from anesthesia on a heating pad and returned to its housing unit. The surgical site was evaluated daily for the duration of the study, and any signs of swelling, discoloration, or herniation at the operative site were recorded. One group of four animals implanted with each device was sacrificed at one, two, four, and eight weeks post surgery. On the planned necropsy date, each rat was anesthetized. At the time of sacrifice, the surgical site and an equal amount of surrounding native tissue were collected for histologic examination.

Immediately after the animal was killed, the defect site along with an equal amount of adjacent native tissue was excised, mounted on a fixed support structure, and placed in 10% neutral buffered formalin. The specimen was then sectioned through its entire thickness and length, including generous amounts of the adjacent normal body wall. The tissue was embedded in paraffin, and mounted on glass slides. The tissue was stained with either hematoxylin and eosin or Masson's trichrome before coverslipping. Macroscopic patterns, i.e. device shrinkage and evaluation of the presence or absence of the test article's anti-adhesive poly L-Lactide-co ε-Caprolactone layer, were determined by gross examination. The thickness of cellular infiltrate was determined microscopically by measuring the magnitude of the cellular infiltrate from the device's inner surface to the edge of infiltrating neotissue within the device. Neotissue formation within the devices was evaluated qualitatively. Histopathologic analysis included evaluation of (1) the amount of cellular infiltration, (2) the presence or absence of multinucleate giant cells, (3) vascularity, and (4) the degree of organization of the replacement connective tissue.

All of the treated animals (n=32) recovered normally post-surgically without signs of an adverse response to the procedure.

Both macroporous synthetic surgical mesh materials tested showed a robust cellular infiltrate within the first week after surgery. The PROCEED™-treated defect sites showed a complete infiltration throughout the device, while coated reinforced composite devices limited cellular infiltration from the periphery and defect site across the device's inner surface. The coated reinforced composite mesh device's anti-adhesive Poly L-Lactide-co ε-Caprolactone outer layer prevented cellular infiltration and adhesion to the overlying tissue, despite the formation of a well-defined fibrous connective tissue layer. High levels of mononuclear cell infiltrations were observed in either device after one week, with the PROCEED™-treated sites appearing to display a slightly higher level than that of coated reinforced composite devices—treated sites. This was not evident at the later timepoints. Generally, both devices elicited a strong angiogenic response and the presence of multinucleate giant cells was observed. Both devices showed an increasing amount of connective tissue formation over time and the deposition of extracellular matrix. This was particularly evident in the coated reinforced composite mesh devices-treated sites (FIG. 12) at 8 weeks, which may have been facilitated by a higher level of porosity and mesh thickness compared to PROCEED™-treated sites.

In FIG. 12, the host response to the coated reinforced composite mesh device after eight week showed a dense cellular infiltration into the device's inner layer directly facing the defect site. A well-defined fibrous connective tissue layer was present across the device's outer surface, which did not lead to cellular infiltration into the device's outer layer due to the presence of the anti-adhesive PolyL-Lactide-co ε-Caprolactone layer. Histologic staining (Masson's Trichrome staining: nuclei: blue/black; muscle, red blood cells, fibrin: red; connective tissue: blue) showed a moderate number of mononuclear cells directly associated with the test device, the moderate formation of connective tissue and the deposition of increasing amounts of extracellular matrix within the center of infiltrated pores. Vascularization was abundant. Multinucleate giant cells were present near the implanted device coated reinforced composite device. Also present were mononuclear cell infiltration, fibrous tissue formation, blood vessel, multinucleate giant.

This study confirms the ability of the coated reinforced composite device to elicit robust tissue ingrowth in a well established rat abdominal wall of hernia repair.

### Example 7

### Use of Composite of Reticulated Elastomeric Matrix 2 with 2-Dimensional Mesh Reinforcement Implanted and a Film of Biocompatible Polymer that Act as Anti-Adhesive Coating in a Rabbit Anti-Adhesion Animal Model

An implantable device formed from one layer of Reticulated Elastomeric Matrix 2 reinforced with the 2-dimensional mesh reinforcement and a film of biocompatible polymer that act as a antiadhesive coating (coated reinforced composite mesh) was made as described in foregoing Example 5. The objective of this animal study was to compare the intra-abdominal adhesion formation of the coated reinforced composite devices to PROCEED™ control device in a rabbit model at 30 days.

A total of 17 New Zealand White Rabbits (Oryctolagus cuniculus) of 3-3.5 kg was used for the study. Laparoscopic examination was conducted at the end of 30 days. Each animal was implanted with two randomly assigned meshes. 10 implants per study arm were implanted in the study. The devices were trimmed interoperatively to 6 cm×5 cm. At the end of 30 days, adhesion assessment was done by laparoscopic assessment points and were evaluated for rate of Adhesion formation (%) per group (presence or absence of adhesions) and the type of adhesion (Filmy, thick, extensive).

Adhesion Scores (Modified Diamond Scale score 0-4) were based on 0=no adhesions, 1=single filmy band, 2=<25% of mesh involved, 3=26-50% of mesh involved and 4=>50% of mesh involved.

The results of the 30 day study are summarized in the table below:

| | Average | | Adhesion Type | | |
|---|---|---|---|---|---|
| | Adhe- | | (number | | |
| | sion | Adhesion | of observations) | | |
| Mesh Group | Score | Rate | FILMY | THICK | EXTENSIVE |
| Coated | 0.1 | 11% | 1 | 0 | 0 |
| reinforced | | | | | |
| composite | | | | | |
| PROCEED ™ | 0.7 | 30% | 0 | 2 | 1 |

Coated reinforced composite mesh device was associated with only 1 filmy adhesion in this series and had the lower adhesion score compared to PROCEED™ mesh that was associated with adhesions in 30% of samples and had either thick or extensive adhesions showing. The results from coated reinforced composite mesh validate the design of the anti-adhesion coating for intraperitoneal placement by the use of poly L-Lactide-co ε-Caprolactone layer in this established animal model.

### Example 8

### Fabrication of Coated Composite Made from Reticulated Elastomeric Matrix Reinforced with 2-Dimensional Mesh Reinforcement Using Polycarbonate Polyurethane Films

ChronoflexAR™ (a solution of polycarbonate polyurethane in DMAC and made by Cardiotech) may be used to make a permanent anti-adhesive coating. The Chronoflex is poured into the trough and spread evenly in the trough using a blade. The trough is heated in a vacuum oven pre-heated to 65° C. under vacuum of 15″ Hg for 1 hour. followed by full vacuum of 30″ Hg for 3 hours at 65° C. to dry the solvent DMAC. The vacuum oven is cooled to room temperature and a blade is used to remove a film of ChronoflexAR™ of thickens about 100 microns and the peeled film is saved on wax coated paper.

The film is cut to a size of about 12 cm×15 cm and the film is brush coated with more Chronoflex on a Teflon coated plates. A 2.0 mm thick sandwich composite made from Reticulated Elastomeric Matrix reinforced (from foregoing Example 3) with 2-dimensional mesh reinforcement measuring about 12 cm×15 cm may be placed on the brush coated film. The film is patted lightly by hand to ensure good contact with the Reticulated Elastomeric Matrix reinforced sheet (0.9 mm thick) and the film. One mm Shims are placed along the edges of the plate and another Teflon paper lined plate is placed on the top. The assembly is placed in the the vacuum oven (pre heated to 65° C.) under vacuum of 30″ Hg for 2 to 3 hours to dry and remove the solvent DMAC. It is cooled and removed from the vacuum oven.

### Example 9

### Fabrication of Coated Composite Made from Reticulated Elastomeric Matrix Reinforced with 2-Dimensional Mesh Reinforcement Using Polycarbonate Polyurethane Films

The process of making a coated composite may be repeated except that the 2.0 mm thick sandwich composite made from Reticulated Elastomeric Matrix reinforced with 2 dimensional mesh reinforcement may be made with ChronoflexAR™ adhesive instead of Silicone adhesive, Nusil. The ChronoflexAR™ is applied to the 2 dimensional PP mesh using Teflon coated plates, and the coated PP mesh may be brought into contact with the Reticulated Elastomeric Matrix, the preform of PP Mesh and Reticulated Elastomeric Matrix may be held under constraint and the solvent DMAC may be dried and removed using a vacuum oven at 65° C. for 3 to 4 hours. The The ChronoflexAR™ film may be attached in the same fashion as in foregoing Example 8. This may create a Chronoflex adhesive bonded composite of Reticulated Elastomeric Matrix with PP mesh.

The ChronoflexAR™ film (the coating to act as anti-adhesion barrier) may be made and may be attached to the Chronoflex adhesive bonded composite of Reticulated Elastomeric Matrix with PP mesh, in the same fashion as the Chronoflex film was attached in Example 8.

### Example 10

### Fabrication of Composite Made from One Layer of Reticulated Elastomeric Matrix Reinforced with 2 Dimensional Mesh Reinforcement and a Film of Biocompatible Polymer that Act as Anti-Adhesive Coating

Another process for manufacturing implantable composite device with anti-adhesive coating is described next. Reticulated Elastomeric Matrix 2 was made following the procedures described in Example 2. Implantable devices, shaped as rectangular sheets having approximately dimensions of 150 mm in length, 120 mm in width and 0.9 mm in thickness, were cut by machining from Reticulated Elastomeric Matrix 2. One sheet or substrate was machined.

A knitted polypropylene monofilament fibers (diameters approximately 0.10 mm) in a mesh configuration having a thickness of approximately 0.41 mm and a Mesh Areal Density of 46-54 g/m² is used as the 2 dimensional mesh reinforcement. The PP mesh, is sized similar to the machined Reticulated Elastomeric Matrix.

A Silicone adhesive (Nusil™ MED2-4213) is used to bond the PP mesh to the single sheet or substrate of Reticulated Elastomeric Matrix.

The anti-adhesion coating materials is (a copolymer of poly (L-lactide co ε-caprolactone) in the molar ratio 70:30) and also known as cap/lac 30/70 provides an flexible and coating designed to minimize adhesions while biodegrading within a year. The inherent viscosity of the cap/lac pellets were between 1.2 and 1.8 dl/g and its melting point is about 112° C.

A film of the copolymer is made via a compression molding process to convert cap/lac pellets (dried for a minimum of 8 hours) into a flat sheet with typical thickness of 110 to 120 microns utilizing a Wabash Genesis Series Heated Compression Press G30H-18-CLX. The forming process involves a series of progressively higher temperature and pressure settings ranging from 120 C/<1 Ton to 140 C/30 Tons with a platen gap of 0.004″. Formed film sheets are allowed to cool under ambient conditions to 50 C prior to further processing.

The cap/lac 30/70 film sheet is then re-melted and bonded to the PP mesh (previously treated with corona discharge in the same way described in Example 3) using precision-ground stainless steel tooling to apply uniform compressive loads to both surfaces. An inert convection oven (using nitrogen) is then used to provide sufficient heat (140 C for 20 minutes) to allow the cap/lac film to flow approximately 0.2 mm into the mesh grid without migrating to the other side.

The final coated composite is created by bonding the cap/lac film-PP mesh construct to the reticulated elastomeric matrix using silicone adhesive. This is achieved by embedding the mesh side of the film/mesh construct into a thin film (0.254 mm) of silicone adhesive in order to transfer sufficient adhesive to the mesh necessary to engage the reticulated elastomeric matrix sheet and maintain a fully open structure at the interface. Fixed-gap tooling similar to that used for the film/mesh construct is used in conjunction with convective heat (100 C for 30 minutes) to cure the silicone.

The final coated composite is created by bonding the cap/lac film-PP mesh construct to the reticulated elastomeric matrix using silicone adhesive. This is achieved by embedding the mesh side of the film/mesh construct into a thin film (0.254 mm) of silicone adhesive in order to transfer sufficient adhesive to the mesh necessary to engage the reticulated elastomeric matrix sheet and maintain a fully open structure at the interface. Fixed-gap tooling similar to that used for the film/mesh construct is used in conjunction with convective heat (100 C for 30 minutes) to cure the silicone. The implantable composite devices with anti-adhesive coating are the trimmed to final size and washed in sonicating baths containing isopropyl alcohol.

The thickness of the composite is approximately 1 mm. The average coat weight of the silicone adhesive was measured to be about 4 milligram/cm² to about 10 milligram/cm² of the surface of the elastomeric matrix.

Each implantable composite devices with anti-adhesive coating, incorporating the PP mesh and the cap/lac film was tested for suture retention strength (SRS), tensile break strength (TBS), and burst strength (BS) using the test methods described in Example 3. An average SRS value of 35±6 Newtons was obtained from testing these implantable composite devices with anti-adhesive coating. An average TBS value of 212±25 Newtons was obtained from testing these implantable composite devices with anti-adhesive coating. An average BS value of 326±51 Newtons was obtained from testing these implantable composite devices with anti-adhesive coating.

Permeability of the implantable composite devices after removal of the cap/lac layer by contacting with chloroform were approximately equivalent to that of the substrate of the Reticulated Elastomeric Matrix 2.

### Example 11

### Fabrication of Composite Made from One Layer of Reticulated Elastomeric Matrix Reinforced with 2 Dimensional Mesh Reinforcement and a Film of Biocompatible Polymer that Act as Anti-Adhesive Coating

Another process for manufacturing implantable composite device with anti-adhesive coating is described next. Reticulated Elastomeric Matrix 2 was made following the procedures described in Example 10 with changes to the composition to fabrication as follows:

The cap/lac 30/70 film sheet is then re-melted and bonded to the PP mesh (previously treated with corona discharge in the same way described in Example 3) using precise and controlled application of compressive force/displacement and heat to engage only one side of the PP mesh. A compression molder (Wabash Genesis Series Heated Compression Press G30H-18-CLX) is used for this purpose and the cap/lac film is melted with the compression molding platen at a temperature of about 120° C. and the film is heated between 10 to 20 minutes. The platens are rapidly cooled using circulating cold water and opened (releasing the compression pressure) for removal of the cap/lac film-PP mesh construct only after the platen temperatures drop to below 70 C. Shims are used to control the thickness of the cap/lac film-PP mesh construct. The final coated composite is created by bonding the cap/lac film-PP mesh construct to the reticulated elastomeric matrix using the silicone adhesive. The process of bonding the the reticulated elastomeric matrix sheet or substrate via application of a thin film of silicone adhesive to the mesh side of the cap/lac film-PP mesh construct follows similar process (80° C. for 2 hours) conditions of application and heat curing of silicone and at the end of the silicone curing process, implantable composite device with anti-adhesive coating is obtained. The implantable composite devices with anti-adhesive coating are washed in using sonicating baths containing isopropyl alcohol.

### Example 12

### Fabrication of Coated Composite Made from Reticulated Elastomeric Matrix Using cap/lac Copolymer Films

Following steps similar to the ones described in making the Chronoflex film from solution casting described in Example 8, using a 20% solution of a copolymer of poly (L-lactide co ε-caprolactone) in the molar ratio 70:30) (also known as cap/lac 30/70) in DMAC.

Reticulated elastomeric matrix was coated with a 10% solution of cap/lac 30/70 in DMAC. The coated matrix and the cap/lac film was melt bonded between teflon coated sheet placed in a vacuum oven that was held at 75 C for 45 minutes followed by 120 C for 90 minutes and cooled to room temperature before taking out the cap/lac film coated reticulated elastomeric matrix sheet.

### Example 13

### Fabrication of Coated Composite Made from Reticulated Elastomeric Matrix Using cap/lac Copolymer Films

The process followed here was similar except the cap/lac film was made by compression molding as described in Example 10 and melt bonded to reticulated elastomeric matrix using the compression molder described in Example 10 and using a composite fabrication or consolidation step of 120° C. for 15 minutes before cooling the platens of the compression molder was cooled by cold water and removing the coated Reticulated Elastomeric Matrix

### Example 14

### Another Exemplary Embodiment of Device

Another exemplary embodiment may be in the form of a composite surgical mesh prepared using two layers of an exemplary reticulated elastomeric matrix. An exemplary mesh (knitted polypropylene monofilament fibers, Biomedical Structure PPM-5) is sandwiched between the two layers. The exemplary polypropylene mesh may have a thickness of about 0.4 mm. A Silicone adhesive (commercially available as NuSil MED2-4213) is used to bond the substrates. The exemplary embodiment of the device may have a thickness of 2.0±0.3 mm.

The two layers of reticulated elastomeric matrix for this exemplary embodiment is prepared from a block of polyurethane matrix having the following composition:

| | | Parts by | Preferred |
|---|---|---|---|
| | | Weight | Parts |
| Description | Chemical | Range | Level |
| Component A | | | |
| Isocyanate | Mondur MRS-20 * | 43.47-47.81 | 45.64 |
| Component B1 | Per MI9000002 | 107.80-109.80 | 108.80 |
| Polyol Component | POLY-CD ™ CD220 | 100 | 100 |
| Viscosity | Propylene carbonate | 5.80 | 5.80 |
| Depressant | | | |
| Cell Opener | Ortegol 501 | 2.00-4.00 | 3.00 |
| Component C3 | Per MI9000005 | 6.05-8.10 | 7.05 |
| Crosslinker | Glycerin | 0.90-1.10 | 1.00 |
| Blowing Agent | Distilled water | 1.50-1.70 | 1.60 |
| Chain Extender | 1,4 BDO | 1.40-1.60 | 1.50 |
| Surfactant | Tegostab BF 2370 | 1.00-1.40 | 1.20 |
| Surfactant | Tegostab B 8300 | 0.45-0.75 | 0.60 |
| Surfactant | Tegostab B 5055 | 0.45-0.75 | 0.60 |
| Amine Catalyst | Dabco 33LV | 0.25-0.55 | 0.40 |
| Amine Catalyst | A-133 | 0.10-0.25 | 0.15 |
| Isocyanate Index | | 1.00 | 1.00 |

The exemplary isocyanate component may be Mondur MRS-20 (commercially available from Bayer) which may includes 30 to 40% by weight of 2,4′ and 2,2′ Diphenylmethane diisocyanate (MDI) mixed isomers (CAS No. 26447-40-5), 30 to 40% by weight of 4,4′-Diphenylmethane diisocyanate (MDI) (CAS No. 101-68-8) and 20 to 30% by weight of Polymeric diphenylmethane diisocyanate (pMDI) (CAS No. 9016-87-9).

The block of polyurethane matrix is machined into thin slices, at a thickness of about 0.9 mm each and an adhesive is applied to the polypropylene knitted mesh in a controlled manner, the composite mesh is assembled in a tri-layer structure and the layers are cured. Individual implants are trimmed to size. The exemplary device may be in a rectangular shape having a length of 100±2 mm and a width of 50±2 mm. The entire mesh is then washed to remove any unreacted processing aids or other impurities. An exemplary process flow diagram is shown in Attachment G.

The device of Example 14 was tested for biocompatibility according to ISO 10993-1, for an implant device contacting tissue/bone for a permanent duration. All results were passing.

Biocompatibility Testing Results

| Biological Test | Result |
|---|---|
| Cytotoxicity: MEM Elution | Non-cytotoxic (Grade 0) |
| Sensitization: Kligman Maximization | Grade I - weak allergic potential |
| Intracutaneous injection | Negligible irritant |
| Systemic injection | Negative |
| Subchronic toxicity: 14-day | Non-toxic |
| Genotoxicity: Ames mutagenicity | Non-mutagenic |
| Genotoxicity: Chromosomal aberration | Non-clastogenic |
| Genotoxicity: Bone marrow | Non-clastogenic |
| micronucleus | |
| Short-term intramuscular implant - 2 | No reaction (Rating = 2.2) |
| weeks | |
| Short-term intramuscular implant - 12 | No reaction (Rating = 0.6) |
| weeks | |
| Material-mediated pyrogenicity | Non-pyrogenic |

Real-time degradation testing of the device of Example 14 was performed per ISO 10993-13 to confirm the material's biostability by identifying and quantifying any degradation products released. Testing was performed by real-time aging finished, sterile samples at 37±1° C. in a simulated hydrolytic degradation solution, Sorenson's buffer. Samples were tested for mass loss as an indicator of degradation and swellability as an indicator of change in cross-linking density. The pH of the solutions was monitored as an additional indicator of degradation.

Testing at one and three months on the finished device of Example 14 has demonstrated no evidence of degradation based on observable mass loss, dimensional changes, and pH.
Mass loss at 1 and 3 months: 0.58%
pH change: 0.02 pH change
area change and % decrease in thickness: 0.2% at 1 month and 0.4% at 3 months 1.48% at 1 month and 0.52% at 3 months
   An additional analysis for the presence of silicone was performed on the solution at 3 months. Analysis of the degradation solution at three months showed no detection of any silicone in the solution at a detection limit of 5 ppm.

In addition, real-time degradation data were submitted through 6 months on the reticulated elastomeric matrix of Example 14. The matrix is biostable in particular due to the polycarbonate urethane cross-linked chemistry. The data demonstrated no material degradation, including no detection of MDA in the buffer solution used during the aging.

Extractable testing from the device of Example 14 was performed per ISO 10993-12, Sample preparation and reference materials, to examine the type and amount of leachable material that has the potential of being released from the implant. Testing was performed using finished, sterile samples. One sample was cured for ˜½ the normal duration, and the other sample was cured the remaining time post-sterilization. Both of these cases are considered worst case for extractables (in the case of incomplete curing). Samples were washed with isopropyl alcohol in an ultrasonic bath. The wash solution was analyzed for volatile organic compounds and semi-volatile compounds. There were no volatile organic compounds and only low levels (<80 ppm) of semi-volatile compounds detected that were attributable to the test article. The levels are significantly lower than the accepted levels for humans thus demonstrating that the device of Example 14 does not result in toxic leachable substances.

### Suture Retention Strength

Purpose: The purpose of this testing was to demonstrate that the device of Example 14, as manufactured and sterilized, met the specification for suture retention strength (SRS). SRS testing determines the maximum resistance provided by the mesh as a standard size suture (2-0 polyester) is pulled through the mesh causing it to fail.

Acceptance criteria: The minimum suture retention strength must be ≧15 N.

A specification of ≧15 N was chosen based on testing of the Ethicon Mersilene Mesh, which demonstrated a suture retention strength of 14.3±0.9 N.

Number of samples: Thirty (30) finished, sterile samples and thirty (30) finished, sterile samples that were accelerated aged for the equivalent of one year were tested.

Test description: Testing was performed using an Instron Tester with Series IX software. The gauge length (distance between the jaws of the Instron) was set to a pre-determined value. A 2-0 braided polyester suture was inserted into one end of the mesh using a needle. A loop was formed by the two ends of the suture strands. The suture must to be attached to the mesh 3 to 5 mm from the edge of the mesh and preferably towards the middle of the mesh width. The mesh and the free ends of the suture were enclosed in the opposing grips. Samples were pulled to failure at a rate of 100 mm/min. The load exerted on the sample and the displacement between the jaws holding the sample was monitored until the sample failed. Using the Series IX software, maximum force was calculated based on the measurements taken and reported.

Results:

| | | Avg Max | Std | | | LTL |
|---|---|---|---|---|---|---|
| | n | Force [N] | Dev [N] | Min [N] | Max [N] | 95%/95% [N] |
| T₀ | 30 | 26.99 | 4.03 | 19.59 | 35.53 | 18 |
| T₁ | 30 | 24.89 | 4.18 | 16.82 | 33.26 | 16 |
| Spec | | ≧15 N | | | | |

Conclusion: These results demonstrated that the finished, sterile device of Example 14 met the minimum suture retention strength specification of 15 N. All devices met the acceptance criteria at Time 0 and after one-year accelerated aging. With 30 samples tested, it was concluded that there was a 95% confidence and 95% reliability that the devices met the suture retention strength specification at both Time 0 and after one-year accelerated aging based on the data. These results are considered equivalent to predicate devices and acceptable for clinical use of the device.

### Break Strength

Purpose: The purpose of this testing was to demonstrate that the device of Example 14, as manufactured and sterilized, meets the specification for mesh tensile strength by measuring the tensile break strength (at maximum load).

Acceptance criteria: The minimum tensile break strength must be ≧140 N.

A specification of ≧140 N was chosen based on testing of the Ethicon Mersilene Mesh, which demonstrated a tensile break strength of 137.6±9.4 N.

Number of samples: Thirty (30) finished, sterile samples and thirty (30) finished, sterile samples that were accelerated aged for the equivalent of one year were tested.

Test description: Testing was performed using an Instron Tester with Series IX software. Break strength testing was conducted following the methodology outlined in 3574-05 Test E. The width and thickness of the sample were measured using calipers/thickness gauge, and the gauge length (distance between the jaws of the Instron) was set to a pre-determined value. Samples were pulled to failure at a rate of 100 mm/min. The load exerted on the sample and the displacement between the jaws holding the sample were monitored until the sample failed. Using the Series IX software, maximum force was calculated based on the measurements taken and reported.

Results:

| | | Avg Max | Std | Min | Max | LTL |
|---|---|---|---|---|---|---|
| | n | Force [N] | Dev [N] | [N] | [N] | 95%/95% [N] |
| T₀ | 30 | 215.71 | 24.73 | 171.17 | 256.76 | 161 |
| T_{1-year, acc} | 30 | 213.41 | 20.51 | 172.41 | 254.32 | 168 |
| Spec | | ≧140 N | | | | |

Conclusion: These results demonstrated that the finished, sterile device of Example 14 met the minimum break strength specification of 140 N. All devices met the acceptance criteria at Time 0 and after one-year accelerated aging.

With 30 samples tested, it was concluded that there was a 95% confidence and 95% reliability that devices met the mesh break strength specification at both Time 0 and after one-year accelerated aging based on the data. These results were considered equivalent to predicate devices and acceptable for clinical use of the device.

### Tear Strength

Purpose: The purpose of this testing was to determine the tear resistance properties of the device of Example 14, as manufactured and sterilized, by measuring the maximum load (tear strength).

Acceptance criteria: The minimum tear strength must be ≧10 N.

A specification of ≧10 N was chosen based on testing of the Ethicon Mersilene Mesh, which demonstrated a tear strength of 9.4±0.9 N.

Number of samples: Thirty (30) finished, sterile samples and thirty (30) finished, sterile samples that were accelerated aged for the equivalent of one year were tested.

Test description: Testing was performed using an Instron Tester with Series IX software. Tear resistance testing was conducted following the methodology outlined in ASTM D3574-05, Test F. A 9 mm slit was cut along the center line of the width of the sample, parallel to the length of the sample. The width and thickness of the sample were measured using calipers/thickness gauge, and one side of the tear was secured in each grip. Samples were pulled to failure at a rate of 101.6 mm/min. The load exerted on the sample and the displacement between the jaws holding the sample were monitored until the sample failed. Using the Series IX software, maximum force was calculated based on the measurements taken and reported.

Results:

| | | Avg Tear | Std | | Max | LTL |
|---|---|---|---|---|---|---|
| | n | Strength [N] | Dev [N] | Min [N] | [N] | 95%/95% [N] |
| T₀ | 30 | 19.90 | 2.96 | 15.32 | 26.32 | 13 |
| T_{1-year, acc} | 30 | 21.98 | 3.89 | 15.99 | 30.46 | 13 |
| Spec | | ≧10 N | | | | |

Conclusion: These results demonstrated that the finished, sterile device of Example 14 met the minimum tear strength specification of 10 N. All devices met the acceptance criteria at Time 0 and after one-year accelerated aging.

With 30 samples tested, it was concluded that there was 95% confidence and 95% reliability that devices met the mesh tear strength specification at both Time 0 and after one-year accelerated aging based on the data. These results were considered equivalent to predicate devices and acceptable for clinical use of the device.

### Ball Burst

Purpose: The purpose of this testing was to determine the ball burst strength of the device of Example 14, as manufactured and sterilized, by measuring the maximum load at yield or rupture of the device.

Acceptance criteria: The minimum ball burst strength must be ≧180 N.

A specification of ≧180 N was chosen based on testing of the Ethicon Mersilene Mesh, which demonstrated a ball burst strength of 179.1±3.6 N.

Number of samples: Thirty (30) finished, sterile samples and thirty (30) finished, sterile samples that were accelerated aged for the equivalent of one year were tested.

Test description: Testing was performed using an Instron Tester with Series IX software. Ball burst testing was conducted following the methodology outlined in ASTM 3787-07. A ball burst fixture with a 1″ (25.4 mm) ball was used. The ball was pushed through the mesh at a rate of 102 mm/min. The load exerted on the sample and the displacement between the jaws holding the sample were monitored until the sample failed (yielded or ruptured). Using the Series IX software, maximum force was calculated based on the measurements taken and reported.

Results:

| | | Avg Burst | | | | |
|---|---|---|---|---|---|---|
| | | Strength | Std | | Max | LTL |
| | n | [N] | Dev [N] | Min [N] | [N] | 95%/95% [N] |
| T₀ | 30 | 352.23 | 51.19 | 271.50 | 437.84 | 239 |
| T_{1-year, acc} | 30 | 330.84 | 52.29 | 225.90 | 429.75 | 215 |
| Spec | | ≧180 N | | | | |

Conclusion: These results demonstrated that the finished, sterile device of Example 14 met the minimum ball burst strength specification of 180 N. All devices met the acceptance criteria at Time 0 and after one-year accelerated aging.

With 30 samples tested, it was concluded that there was 95% confidence and 95% reliability that devices met the ball burst strength specification at both Time 0 and after one-year accelerated aging based on the data. These results were considered equivalent to predicate devices and acceptable for clinical use of the device.

### Permeability

Purpose: The purpose of this testing was to determine the liquid permeability of the device of Example 14, as manufactured and sterilized, by measuring the ability of the composite device to allow fluid flow through the material.

Acceptance criteria: A specification of >60 Darcy was chosen based on the current process capability of the manufacturing process. The specification was confirmed as acceptable because devices were used in the in-vivo study in the rat abdominal wall. This study demonstrated tissue in-growth throughout the entire cross-section of the mesh.

Number of samples: Thirty (30) finished, sterile samples and thirty (30) finished, sterile samples that were accelerated aged for the equivalent of one year were tested.

Test description: Testing was performed using an Automated Liquid Permeameter with Capwin Automated Liquid Permeameter software. A 14 mm disc (2 mm thickness) was cut from the mesh and tested.

Results:

| | | Avg | | | | LTL |
|---|---|---|---|---|---|---|
| | | Permeablity | Std Dev | Min | Max | 95%/95% |
| | n | [Darcy] | [Darcy] | [Darcy] | [Darcy] | [Darcy] |
| T₀ | 30 | 261.56 | 65.15 | 187.82 | 466.90 | 117 |
| T_{1-year, acc} | 30 | 258.27 | 70.46 | 133.38 | 417.28 | 102 |
| Spec | | ≧60 Darcy | | | | |

Conclusion: These results demonstrated that the finished, sterile device of Example 14 met the minimum permeability specification of 60 Darcy. All devices met the acceptance criteria at Time 0 and after one-year accelerated aging.

With 30 samples tested, it was concluded that there was a 95% confidence and 95% reliability that devices met the permeability specification at both Time 0 and after one-year accelerated aging based on the data. These results are considered acceptable for clinical use of the device.

### Peel Strength

Purpose: The purpose of this testing was to determine the peel strength of the device of Example 14, as manufactured and sterilized, by measuring the load required to separate the adhered surfaces.

Acceptance criteria: There were no acceptance criteria for this testing. Characterization test only.

Number of samples: Thirteen (13) 20 mm wide samples and thirteen (13) 16 mm wide samples cut from two (2) finished, sterile devices and thirty-six (36) 20 mm wide samples cut from two (2) finished, sterile devices that were accelerated aged for the equivalent of one year were tested.

Test description: Testing was performed using an Instron Tester with Series IX software. Peel testing was conducted following the methodology outlined in ASTM D1876. Special samples were prepared, with the total length of the sample >40 mm, approximately 20 mm of which was non-bonded, resulting in two tabs of the device of Example 14 at least 20 mm long. These tabs were not bonded to the polypropylene mesh and served as “pull tabs” to accommodate the Instron grips. One of two tabs was gripped in the top grip and the other in the bottom grip of the Instron before the test was started. The ends were pulled at a rate of 25.4 mm/min. The load exerted on the sample and the displacement between the jaws holding the sample were monitored until the sample failed. Using the Series IX software, maximum force was calculated based on the measurements taken and reported.

Results:

| | | | | Min | |
|---|---|---|---|---|---|
| | n | Avg Peel [N] | Std Dev [N] | [N] | Max [N] |
| T₀ - 20 mm | 13 | 4.97 | 0.73 | 3.08 | 6.10 |
| T₀- 16 mm | 13 | 3.78 | 0.38 | 3.17 | 4.50 |
| T_{1-year, acc} - 16 mm | 36 | 5.59 | 1.55 | 3.79 | 9.47 |

Conclusion: This test, in which a layer of the device of Example 14 was deliberately peeled from the polypropylene mesh in specially constructed peel test samples, was a characterization test which was solely used to assess the manufacturing process. Clinically, there are no analogous peel forces placed on the mesh, either during the procedure or post-implantation.

This test demonstrated that the manufacturing process successfully adhered the layers of the composite device. The predominant mode of failure was substrate failure (cohesive), meaning that failure of the matrix occurred, which indicated good bond strength of the adhesive. All the other mechanical tests performed on the devices, like ball burst and tensile testing, did not show any signs of layer delamination during testing. Therefore, it can be concluded that the SMNR composite was adequately bonded.

### Stiffness

Purpose: The purpose of this testing was to determine the tensile stiffness of the device of Example 14, as manufactured and sterilized, as calculated from the tensile testing results.

Acceptance criteria: Equivalent to predicate devices. Results from predicate devices tested were included in the test report.

Number of samples: Thirty (30) finished, sterile samples and thirty (30) finished, sterile samples that were accelerated aged for the equivalent of one year were tested.

Test description: Stiffness was calculated using the slope of the load vs. % strain graph.

Results:

| Sample | n | Avg Stiffness [N/mm] |
|---|---|---|
| T₀ MD | 30 | 0.32 ± 0.05 |
| T_{1-year, acc} MD | 30 | 0.29 ± 0.02 |
| Mersilene, MD | 10 | 0.56 ± 0.01 |
| Mersilene, CMD | 10 | 0.25 ± 0.01 |
| Ultrapro, MD | 10 | 1.77 ± 0.09 |

MD = Machine Direction
CMD = Counter-Machine Direction

Conclusion: This testing demonstrated that the stiffness values of the device of Example 14 were bounded by the corresponding values for Mersilene CMD (lower bound), and Ultrapro MD (upper bound).

All devices met the acceptance criteria at Time 0 and after one-year accelerated aging. The device of Example 14 was equivalent to other meshes at the device level in terms of whole device stiffness. These results were considered acceptable for clinical use of the device.

An in-vivo animal study was conducted using the exemplary device of Example 14 in a rat abdominal wall model to assess the healing response to the mesh. The animals were subjected to replacement of an experimentally-induced body wall defect (1 cm×1 cm) with the a down-sized version of the device of Example 14. The device showed a well-tolerated, long term histomorphologic response in the rat abdominal wall model, with good integration with surrounding tissue, minimal foreign body response, and no evidence of device degradation or adjacent tissue necrosis.

### Rat Abdominal Wall Study

A study was performed in a rat body wall repair model to determine the histomorphological tissue response to the device of Example 14.

### Methods

Twenty-four (24) skeletally mature, male, 6-8 weeks old, Sprague-Dawley rats, weighing between 300 and 500 grams, were used as experimental subjects. The animals were divided into six test groups sacrificed at the following time points: 1 week, 2 weeks, 4 weeks, 8 weeks, 16 weeks and 26 weeks. The study was conducted using a well-established rat body wall model (See Valentin et al., “Extracellular matrix bioscaffolds for orthopedic applications. A comparative histologic study.” J Bone Joint Surg Am. 2006 December; 88: 2673-86). Each rat was subjected to removal of a 1 cm×1 cm portion of the ventral lateral abdominal wall and replacement with the exemplary device of Example 14 having a modified configuration for use in the rat model. The devices was downsized to 1 cm×1 cm. The thickness remained 2 mm. These meshes were not washed post-processing, representing a worst case for material biocompatibility assessment in this animal model.

Following the surgical repairs, all rats were sacrificed following the schedule above and histological analysis of the repair was conducted. Microscopic evaluations included the semi-quantitative determination of the presence of the test article, angiogenesis, cellular infiltration, multinucleate giant cells, a fibrous connective tissue layer surrounding the device and host neo-ECM deposition. In addition, measurements (length and width) were taken of devices implanted for 26 weeks.

### Results

### Gross Evaluation

At sacrifice, each implant was evaluated macroscopically for gross evidence of healing, suture encapsulation, loose body and inflammatory reactions. Gross evaluation of the implants at all time points showed a smooth connective facial covering with no signs of degradation or evidence of adjacent tissue necrosis. It was observed that the amount and degree of fibrous connective tissue deposition and the number of multinucleate giant cells is stable after approximately 1-2 months post surgery. Cranial-caudal and the medial-laterial dimensions of the device was measured at 26 weeks. The results of the measurements are shown in the table below.

SMNR 26 Weeks: Measurements

| | cranial- | medial- |
|---|---|---|
| | caudal | lateral |
| SAMPLE | (cm) | (cm) |
| 26 WEEK-1 | 1.00 | 0.80 |
| 26 WEEK-2 | 1.00 | 0.90 |
| 26 WEEK-3 | 1.00 | 0.90 |
| 26 WEEK-4 | 1.00 | 0.80 |
| AVERAGE | 1.00 | 0.85 |
| Pre-Implant | 1.0 | 1.00 |

The implant material appeared unchanged throughout the study period. The device of Example 14 showed a well-tolerated, long term histomorphologic response in the rat abdominal wall model, with good integration with surrounding tissue, minimal foreign body response, and no evidence of device degradation or adjacent tissue necrosis. It was observed that mononuclear cell infiltration accompanied by the formation of increasingly organized connective tissue within and surrounding the test article. Vascularization and connective tissue were observed within and surrounding the test article. Most multinucleate giants cells were seen adjacent to implanted device material. Multinucleate giant cells increased from week 1 to week 2 and then stabilized. The level of cellular infiltrate, angiogenesis, multinucleate giant cells, fibrous CT surrounding test article, and amount of connective tissue was visually assessed using a microscope with the following scale:
“−” decrease in the total amount
“+” some increase in the total amount
“++” more increase in the total amount
“+++” significant increase in the total amount
   The results of the microscopic evaluations are shown in the table below.

| | | | | Fibrous CT | Amount |
|---|---|---|---|---|---|
| Animal ID- | | | Multinucleate | Surrounding Test | Connective |
| Slide/Block | Cellular Infiltrate | Angiogenesis | Giant Cells | Article | Tissue |
| Number* | (−, +, ++, +++) | (+, ++, +++) | (−, +, ++, +++) | (−, +, ++) | (+, ++, +++) |
| 26W1-640 | +++ | +++ | ++ | ++ | +++ |
| 26W1-641 | +++ | +++ | ++ | ++ | +++ |
| 26W1-642 | +++ | +++ | ++ | ++ | +++ |
| 26W1-643 | +++ | +++ | ++ | ++ | +++ |

### Histology

Microscope evaluations at each of the time points are shown in Attachment H at 40× magnification.

After 1 week, a moderate number of mononuclear cells associated with loose connective tissue stroma were present at the site of test article implantation. A thin layer of fibrous connective tissue surrounded the test device. There was intense vascularization throughout the implantation sites. Small numbers of multinucleate giant cells were noted near the device material.

After 2 weeks, a moderate to large number of mononuclear cells associated with denser connective tissue stroma were present at the site of test article implantation. A thicker layer of fibrous connective tissue surrounded the test device; this surrounding connective tissue layer integrated with connective tissue stroma noted within the test device material. There was vascularization throughout the implantation sites, and there was an increase in the presence of multinucleate giant cells, still noted near the device material.

After 4 weeks, the site of test article implantation continued to show intense ononuclear cell infiltrate within a dense connective tissue stroma. A well-defined layer of fibrous connective tissue surrounded the test device; this surrounding connective tissue layer integrated with connective tissue stroma noted within the test device material. There was an increased number of blood vessels, and multinucleate giant cells were still noted near the device material.

After 8 weeks, the site of test article implantation continued to show dense ononuclear cell infiltrate within a dense connective tissue stroma. A well-defined connective tissue layer surrounded the test device; this surrounding connective tissue layer integrated with connective tissue stroma noted within the test device material. There was an increased number of blood vessels, and multinucleate giant cells were still noted near the device material.

After 16 weeks, the site of test article implantation continued to show dense mononuclear cell infiltrate within a more dense connective tissue stroma. A well-defined connective tissue layer surrounded the test device; this surrounding connective tissue layer integrated with connective tissue stroma noted within the test device material. The moderate to dense level of vascularization continued, and multinucleate giant cells were still noted near the device material.

After 26 weeks, the site of test article implantation continued to show dense mononuclear cell infiltrate within the dense connective tissue stroma. A well-defined connective tissue layer surrounded the test device; this surrounding connective tissue layer integrated with connective tissue stroma noted within the test device material. The moderate to dense level of vascularization continued, and multinucleate giant cells were still noted near the device material.

At 26 weeks, the length and width of the mesh were measured. In the cranial-caudal direction, all meshes measured at their original dimension of 1.0 cm. In the medial-lateral direction, minimal contraction was noted with an average dimension of 0.85 cm.

Microscope evaluations at 26 weeks are shown in Attachment I at 4×, 10×, 20× and 40× magnification.

### Conclusion

The host response to the exemplary device of Example 14 showed dense mononuclear cell infiltration accompanied by increasingly organized connective tissue within and surrounding the mesh. The amount of vasculature within the implant increased during the early stages of tissue remodeling and then moderated. The number of multinucleate giant cells increased as a function of time by Week 2, and then stabilized. These multinucleate giant cells were typically seen adjacent to implanted device material, and were noted to be less than historical studies with polypropylene mesh implanted in the same rat abdominal wall model. The graft material was present at all time points evaluated, and there was no necrosis of the host tissue surrounding the implanted devices at any time point. Measurements of graft contracture at the 26 week time point showed minimal contracture of ˜15%.

The device of Example 14 showed a well-tolerated, long term histomorphologic response in the rat abdominal wall model, with good integration with surrounding tissue, minimal foreign body response, and no evidence of device degradation or adjacent tissue necrosis.

The entire disclosure of each and every U.S. patent and patent application, each foreign and international patent publication and each other publication, and each unpublished patent application that is referenced in this specification, or elsewhere in this patent application, is hereby specifically incorporated herein, in its entirety, by the respective specific reference that has been made thereto.

While illustrative embodiments of the invention have been described above, it is understood that many and various modifications will be apparent to those in the relevant art, or may become apparent as the art develops. Any equivalent embodiments are intended to be within the scope of this invention. Indeed, various modifications of the invention in addition to those shown and described therein will become apparent to those skilled in the art from the foregoing description. Such modifications are contemplated as being within the spirit and scope of the invention or inventions disclosed in this specification. All publications cited herein are incorporated by reference in their entirety.

What is claimed:

1. A composite implantable device for promoting tissue ingrowth therein, comprising:
a first biodurable reticulated elastomeric matrix and a second biodurable reticulated elastomeric matrix, said first and second matrices each having a three-dimensional porous structure comprising a continuous network of interconnected and intercommunicating open pores, and
a polymeric surgical mesh comprising a plurality of intersecting one-dimensional reinforcement elements,
wherein said mesh is sandwiched between said first and second matrices and affixed to a face of said first matrix and an opposing face of said second matrix.

2. The composite implantable device of claim 1, wherein said first and second matrices comprises polycarbonate polyurethane or polycarbonate polyurethane-urea.

3. The composite implantable device of claim 2, wherein said first and second matrices are formed from a reaction of a polycarbonate polyol and an isocyanate component comprising a mixture of 2,4′ diphenylmethane diisocyanate and 4,4′ diphenylmethane diisocyanate.

4. The composite implantable device of claim 3, wherein said isocyanate component comprising at least 5% by weight of 2,4′ diphenylmethane diisocyanate.

5. The composite implantable device of claim 1, wherein said mesh comprises an absorbable material.

6. The composite implantable device of claim 5, wherein said mesh comprises at least one selected from the group consisting of a polylactic acid or a poly(lactide ε-caprolactone).

7. The composite implantable device of claim 1, wherein said mesh is non-resorbable.

8. The composite implantable device of claim 7, wherein said mesh comprises a polyester or a polypropylene.

9. The composite implantable device of claim 1, wherein said plurality of one-dimensional reinforcement elements comprises polypropylene monofilament fibers.

10. The composite implantable device of claim 9, said polypropylene monofilament fibers are knitted to form said mesh.

11. The composite implantable device of claim 1, further comprising a polymeric film coating covering said first matrix or said mesh, wherein said coating reduces adhesion of said device to biologic surfaces.

12. The composite implantable device of claim 1, wherein said polymeric film comprises poly (L-lactide co ε-caprolactone).

13. The composite implantable device of claim 1, wherein said mesh is bonded to said first matrix by an adhesive.

14. A method for treating a hernia comprising making an incision into an affected area, placing the composite implantable device of claim 1 onto said affected area, and securing said device to said affected area.

15. A method for manufacturing a composite implantable device comprising the steps of:
preparing a first biodurable reticulated elastomeric matrix and a second biodurable reticulated elastomeric matrix, said first and second matrices each having a three-dimensional porous structure comprising a continuous network of interconnected and intercommunicating open pores,
applying an adhesive to a polymeric surgical mesh, wherein said mesh comprises comprising a plurality of intersecting one-dimensional reinforcement elements, and
affixing said mesh to a face of said first matrix and an opposing face of said second matrix such that said mesh is sandwiched between said first and second matrices.

16. A composite implantable device for promoting tissue ingrowth therein, comprising:
a biodurable reticulated elastomeric matrix having a three-dimensional porous structure comprising a continuous network of interconnected and intercommunicating open pores,
a polymeric surgical mesh comprising a plurality of intersecting one-dimensional reinforcement elements, wherein said mesh is affixed to a face of said matrix, and
a polymeric film coating covering said mesh, wherein said coating reduces adhesion of said device to biologic surfaces.

17. A method for treating a hernia comprising making an incision into an affected area, placing the composite implantable device of claim 16 onto said affected area, and securing said device to said affected area.

18. A method for manufacturing a composite implantable device comprising the steps of:
preparing a biodurable reticulated elastomeric matrix having a three-dimensional porous structure comprising a continuous network of interconnected and intercommunicating open pores,
applying an adhesive to a polymeric surgical mesh, wherein said mesh comprises comprising a plurality of intersecting one-dimensional reinforcement elements,
affixing said mesh to a face of said first matrix, and
covering said mesh with a polymeric film, wherein said film reduces adhesion of said device to biologic surfaces.

19. The method of claim 18, wherein said covering step comprises melt-bonding said polymeric film onto said mesh.
